(19) United States
(12) Patent Application Publication
(10) Pub. No.: US 2010/0087382 A1
(43) Pub. Date: Apr. 8, 2010

(54) Inhibitors of Hepatitis C NS3 Protease

(75) Inventors: Murray D. Bailey, Pierrefonds (CA); Francois Bilodeau, Laval (CA); Pasquale Forgione, Montreal (CA); Vida Gorys, Dollard-des-Ormeaux (CA); Montse Llinas-Brunet, Dollard-des-Ormeaux (CA); Julie Naud, Blainville (CA); Jeffrey O'Meara, Boisbriand (CA); Marc-Andre Poupart, Laval (CA)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/527,200

(22) PCT Filed: Feb. 15, 2008
(86) PCT No.: PCT/CA08/00293

§ 371 Date: Aug. 31, 2009

Related U.S. Application Data
(60) Provisional application No. 60/890,304, filed on Feb. 16, 2007.

(51) Int. Cl. A61K 38/06; C12N 7/04; C07K 5/08; A61P 31/12

(52) U.S. Cl. 514/18; 435/236; 530/331

(57) ABSTRACT

Compounds of formula I:

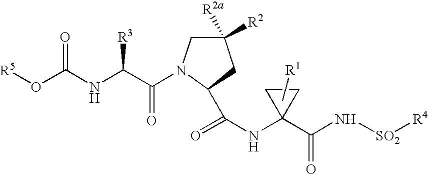

wherein R¹, R², R^{2a}, R³, R⁴ and R⁵ are defined herein, are useful as inhibitors of the HCV NS3 protease.

### RELATED APPLICATIONS

This application claims benefit of U.S. Ser. No. 60/890,304, filed Feb. 16, 2007, which is herein incorporated by reference.

### FIELD OF THE INVENTION

The present invention relates to compounds, compositions and methods for the treatment of hepatitis C virus (HCV) infection. In particular, the present invention provides novel inhibitors of the hepatitis C virus NS3 protease, pharmaceutical compositions containing such compounds and methods for using these compounds in the treatment of HCV infection.

### BACKGROUND OF THE INVENTION

It is estimated that at least 130 million persons worldwide are infected with the hepatitis C virus (HCV). Acute HCV infection progresses to chronic infection in a high number of cases, and, in some infected individuals, chronic infection leads to serious liver diseases such as cirrhosis and hepatocellular carcinoma.

Currently, standard treatment of chronic hepatitis C infection involves administration of pegylated interferon-alpha in combination with ribavirin. However, this therapy is not effective in reducing HCV RNA to undetectable levels in many infected patients and is associated with often intolerable side effects such as fever and other influenza-like symptoms, depression, thrombocytopenia and hemolytic anemia. Furthermore, some HCV-infected patients have co-existing conditions which contraindicate this treatment.

Therefore, a need exists for alternative treatments for hepatitis C viral infection. One possible strategy to address this need is the development of effective antiviral agents which inactivate viral or host cell factors which are essential for viral replication.

HCV is an enveloped positive strand RNA virus in the genus Hepacivirus in the Flaviviridae family. The single strand HCV RNA genome is approximately 9500 nucleotides in length and has a single open reading frame (ORF), flanked by 5′ and 3′ non-translated regions The HCV 5′ non-translated region is 341 nucleotides in length and functions as an internal ribosome entry site for cap-independent translation initiation. The open reading frame encodes a single large polyprotein of about 3000 amino acids which is cleaved at multiple sites by cellular and viral proteases to produce the mature structural and non-structural (NS2, NS3, NS4A, NS4B, NS5A, and NS5B) proteins. The viral NS2/3 protease cleaves at the NS2-NS3 junction; while the viral NS3 protease mediates the cleavages downstream of NS3, at the NS3-NS4A, NS4A-NS4B, NS4B-NS5A and NS5A-NS5B cleavage sites. The NS3 protein also exhibits nucleoside triphosphatase and RNA helicase activities. The NS4A protein acts as a cofactor for the NS3 protease and may also assist in the membrane localization of NS3 and other viral replicase components. Although NS4B and the NS5A phosphoprotein are also likely components of the replicase, their specific roles are unknown. The NS5B protein is the elongation subunit of the HCV replicase possessing RNA-dependent RNA polymerase (RdRp) activity.

The first evidence of the clinical antiviral activity of HCV NS3 protease inhibitors was provided by the results of a two day clinical trial, which indicate that the HCV NS3 protease inhibitor BILN 2061 is effective in rapidly reducing viral loads in patients infected with the hepatitis C virus (Gastroenterology (2004) 127(5): 1347-1355). More recently, in 28- and 14-day clinical trials with the HCV NS3 protease inhibitor VX-950, in combination with pegylated interferon with or without ribavirin, viral load for most HCV patients rapidly decreased to undetectable levels during treatment (Hepatology (2006) 44(4 s1): 532A and 614A).

Inhibitors of the HCV NS3 protease have been described in WO 00/09543 (Boehringer Ingelheim), WO 03/064456 (Boehringer Ingelheim), WO 03/064416 (Boehringer Ingelheim), WO 2004/101602 (Boehringer Ingelheim), WO 2004/101605 (Boehringer Ingelheim), WO 2004/103996 (Boehringer Ingelheim), WO 02/060926 (Bristol-Myers Squibb), WO 03/099316 (Bristol-Myers Squibb), WO 03/099274 (Bristol-Myers Squibb), WO 2004/032827 (Bristol-Myers Squibb), WO 2004/043339 (Bristol-Myers Squibb), WO 2006/122188 (Bristol-Myers Squibb) and WO 2004/113365 (Enanta), herein incorporated by reference.

Inhibitors of the hepatitis C virus NS3 protease of the following generic formula are described in WO 2006/122188, herein incorporated by reference:

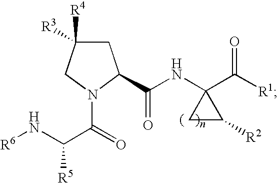

wherein R³ is selected from alkenyl, alkyl, aryl, aryalkyl, cycloalkyl, (cycloalkyl)alkyl, heterocyclyl and heterocyclylalkyl; and R⁴ is selected from hydrogen and hydroxy.

### SUMMARY OF THE INVENTION

The present invention provides novel compounds which show potent activity against hepatitis C virus protease, more particularly the NS3 protease encoded by HCV. Furthermore, the compounds of the invention have activity as inhibitors in a cell-based HCV replication assay. A further advantage of the compounds according to this invention is their specificity for inhibition of the NS3 protease and their low to very low or even non-significant inhibitory activity against other serine proteases such as human leukocyte elastase (HLE) or cysteine proteases such as human liver cathepsin B (Cat B). Further objects of this invention arise for the one skilled in the art from the following description and the examples.

One aspect of the invention provides compounds of formula (I):

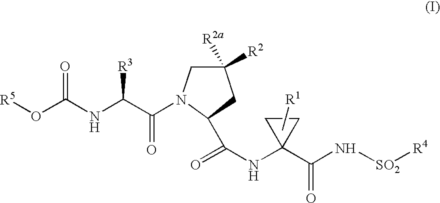

wherein:
R⁵ is selected from:
   (i) (C₁₋₁₀)alkyl optionally substituted with one or more substituents each selected independently from —COOH, —COO(C₁₋₆)alkyl, —OH, halogen, —CN, —OC(═O)(C₁₋₆)alkyl, —O(C₁₋₆)alkyl, —NH₂, —NH(C₁₋₆)alkyl, —N((C₁₋₆)alkyl)₂, —C(═O)NH₂, —C(═O)NH(C₁₋₆)alkyl and —C(═O)N((C₁₋₆)alkyl)₂; and
   (ii) (C₃₋₇)cycloalkyl, (C₃₋₇)cycloalkenyl, (C₃₋₇)cycloalkyl-(C₁₋₄)alkyl- or (C₃₋₇)cycloalkenyl-(C₁₋₄)alkyl-, each optionally substituted with one or more substituents each selected independently from (C₁₋₆)alkyl, (C₂₋₆)alkenyl, (C₂₋₆)alkynyl, —COOH, —COO(C₁₋₆)alkyl, —OH, —O(C₁₋₆)alkyl, —CN, —NH₂, —NH(C₁₋₆)alkyl, —N((C₁₋₆)alkyl)₂, —C(═O)NH₂, —C(═O)NH(C₁₋₆)alkyl and —C(═O)N((C₁₋₆)alkyl)₂;
R³ is (C₁₋₈)alkyl, (C₃₋₇)cycloalkyl or (C₃₋₇)cycloalkyl-(C₁₋₃)alkyl-, each optionally substituted with one or more substituents each independently selected from (C₁₋₆)alkyl, (C₂₋₆)alkenyl, (C₂₋₆)alkynyl, halogen, cyano, —OR³⁰, —SR³⁰, —C(═O)OR³⁰, —C(═O)NH₂, —C(═O)NH(C₁₋₆)alkyl, C(═O)N((C₁₋₆)alkyl)₂, —NH₂, —NH(C₁₋₆)alkyl, —N((C₁₋₆)alkyl)₂, aryl, and aryl(C₁₋₆)alkyl-, wherein R³⁰ is H, (C₁₋₆)alkyl, aryl, or aryl(C₁₋₆)alkyl-;
R² is —O(C₁₋₆)alkyl;
R^{2a} is

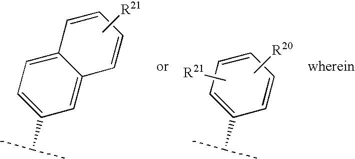

R²⁰ is selected from aryl and Het, each optionally substituted with one or more substituents each independently selected from halogen, cyano, (C₁₋₆)alkyl, (C₁₋₆)haloalkyl, —O(C₁₋₆)alkyl, —S(C₁₋₆)alkyl, —OH, —SH, —NH₂, —NH(C₁₋₆)alkyl, —N((C₁₋₆)alkyl)₂, —NHC(═O)(C₁₋₆)alkyl, —C(═O)NH₂, —C(═O)NH(C₁₋₆)alkyl, —C(═O)N((C₁₋₆)alkyl)₂, —COOH, —C(═O)O(C₁₋₆)alkyl and —SO₂(C₁₋₆)alkyl; and
R²¹ is one to four substituents each independently selected from H, halogen, (C₁₋₆)alkyl, and —O(C₁₋₆)alkyl;
R¹ is (C₁₋₆)alkyl or (C₂₋₆)alkenyl; each of said (C₁₋₆)alkyl, (C₂₋₆)alkenyl being optionally substituted with from one to three halogen substituents; and
R⁴ is (C₃₋₇)cycloalkyl; said (C₃₋₇)cycloalkyl being optionally substituted with (C₁₋₆)alkyl; or R⁴ is —N(R^{N2})R^{N1}, wherein R^{N1} and R^{N2} are each independently selected from H, (C₁₋₆)alkyl and —O—(C₁₋₆)alkyl;
   wherein Het is defined as a 3- to 7-membered heterocycle having 1 to 4 heteroatoms each independently selected from O, N and S, which may be saturated, unsaturated or aromatic, and which is optionally fused to at least one other cycle to form a 4- to 14-membered heteropolycycle having wherever possible 1 to 5 heteroatoms, each independently selected from O, N and S, said heteropolycycle being saturated, unsaturated or aromatic; or a diastereoisomer or tautomer thereof; or a salt thereof.

Another aspect of this invention provides a compound of formula (I) or a pharmaceutically acceptable salt thereof, as a medicament.

Still another aspect of this invention provides a pharmaceutical composition comprising a therapeutically effective amount of a compound of formula (I) or a pharmaceutically acceptable salt thereof; and one or more pharmaceutically acceptable carriers.

According to an embodiment of this aspect, the pharmaceutical composition according to this invention additionally comprises at least one other antiviral agent.

The invention also provides the use of a pharmaceutical composition as described hereinabove for the treatment of a hepatitis C viral infection in a mammal having or at risk of having the infection.

A further aspect of the invention involves a method of treating a hepatitis C viral infection in a mammal having or at risk of having the infection, the method comprising administering to the mammal a therapeutically effective amount of a compound of formula (I), a pharmaceutically acceptable salt thereof, or a composition thereof as described hereinabove.

Another aspect of the invention involves a method of treating a hepatitis C viral infection in a mammal having or at risk of having the infection, the method comprising administering to the mammal a therapeutically effective amount of a combination of a compound of formula (I) or a pharmaceutically acceptable salt thereof, and at least one other antiviral agent; or a composition thereof.

Also within the scope of this invention is the use of a compound of formula (I) as described herein, or a pharmaceutically acceptable salt thereof, for the treatment of a hepatitis C viral infection in a mammal having or at risk of having the infection.

Another aspect of this invention provides the use of a compound of formula (I) as described herein, or a pharmaceutically acceptable salt thereof, for the manufacture of a medicament for the treatment of a hepatitis C viral infection in a mammal having or at risk of having the infection.

An additional aspect of this invention refers to an article of manufacture comprising a composition effective to treat a hepatitis C viral infection; and packaging material comprising a label which indicates that the composition can be used to treat infection by the hepatitis C virus; wherein the composition comprises a compound of formula (I) according to this invention or a pharmaceutically acceptable salt thereof.

Still another aspect of this invention relates to a method of inhibiting the replication of hepatitis C virus comprising exposing the virus to an effective amount of the compound of formula (I), or a salt thereof, under conditions where replication of hepatitis C virus is inhibited.

Further included in the scope of the invention is the use of a compound of formula (I), or a salt thereof, to inhibit the replication of hepatitis C virus.

### DETAILED DESCRIPTION OF THE INVENTION

### Definitions

As used herein, the following definitions apply unless otherwise noted:

The designations “P3, P2, P1 and P1′” as used herein refer to the position of the amino acid residues starting from the N-terminus of the peptide analogs and extending towards and beyond the cleavage site, i.e. the bond in a substrate of the protease enzyme which is normally cleaved by the catalytic action of the protease enzyme. Thus, P3 refers to position 3 from the C-terminal side of the cleavage site, P2 to position 2 from the C-terminal side of the cleavage site, etc. The bond between the P1 and P1′ residues corresponds to the cleavage site. Thus, the P1′ position corresponds to the first position on the N-terminal side of the cleavage site (see Berger A. & Schechter I., Transactions of the Royal Society London series B257, 249-264 (1970), herein incorporated by reference). In the context of the compounds of formula (I) herein described, these positions are as designated in the following formula:

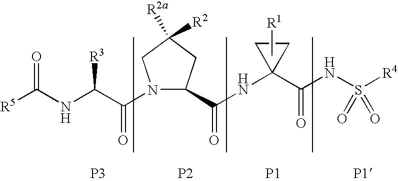

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “(C₁₋ₙ)alkyl” as used herein, wherein n is an integer, either alone or in combination with another radical, is intended to mean acyclic, straight or branched chain alkyl radicals containing from 1 to n carbon atoms. “(C₁₋₆)alkyl” includes, but is not limited to, methyl, ethyl, propyl (n-propyl), butyl (n-butyl), 1-methylethyl (iso-propyl), 1-methylpropyl (sec-butyl), 2-methylpropyl (iso-butyl), 1,1-dimethylethyl (tert-butyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

The term “(C₁₋ₙ)alkylene” as used herein, wherein n is an integer, either alone or in combination with another radical, is intended to mean acyclic, straight or branched chain divalent alkyl radicals containing from 1 to n carbon atoms. “(C₁₋₆)alkylene” includes, but is not limited to, —CH₂—, —CH₂CH₂—,

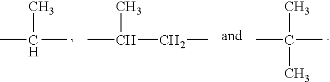

The term “(C₁₋ₙ)alkylidene” as used herein, wherein n is an integer, either alone or in combination with another radical, is intended to mean acyclic, straight or branched chain alkyl radicals containing from 1 to n carbon atoms which are bonded to a molecule or fragment thereof, as a substituent thereof, by a double bond. “(C₁₋₆)alkylidene” includes, but is not limited to, CH₂═, CH₃CH═, CH₃CH₂CH═,

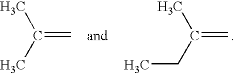

Unless specified otherwise, the term “(C₂₋ₙ)alkylidene” is understood to encompass individual stereoisomers where possible, including but not limited to (E) and (Z) isomers, and mixtures thereof. When a (C₂₋ₙ)alkylidene group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “(C₂₋ₙ)alkenyl”, as used herein, wherein n is an integer, either alone or in combination with another radical, is intended to mean an unsaturated, acyclic straight or branched chain radical containing two to n carbon atoms, at least two of which are bonded to each other by a double bond. Examples of such radicals include, but are not limited to, ethenyl (vinyl), 1-propenyl, 2-propenyl, and 1-butenyl. Unless specified otherwise, the term “(C₂₋ₙ)alkenyl” is understood to encompass individual stereoisomers where possible, including but not limited to (E) and (Z) isomers, and mixtures thereof. When a (C₂₋ₙ) alkenyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “(C₂₋ₙ)alkynyl”, as used herein, wherein n is an integer, either alone or in combination with another radical, is intended to mean an unsaturated, acyclic straight or branched chain radical containing two to n carbon atoms, at least two of which are bonded to each other by a triple bond. Examples of such radicals include, but are not limited to, ethynyl, 1-propynyl, 2-propynyl, and 1-butynyl. When a (C₂₋ₙ)alkynyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “(C₃₋ₘ)cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl.

The term “(C₃₋ₘ)cycloalkyl-(C₁₋ₙ)alkyl-” as used herein, wherein n and m are both integers, either alone or in combination with another radical, is intended to mean an alkyl radical having 1 to n carbon atoms as defined above which is itself substituted with a cycloalkyl radical containing from 3 to m carbon atoms as defined above. Examples of (C₃₋₇)cycloalkyl-(C₁₋₆)alkyl- include, but are not limited to, cyclopropylmethyl, cyclobutylmethyl, cyclopentylmethyl, cyclohexylmethyl, 1-cyclopropylethyl, 2-cyclopropylethyl, 1-cyclobutylethyl, 2-cyclobutylethyl, 1-cyclopentylethyl, 2-cyclopentylethyl, 1-cyclohexylethyl and 2-cyclohexylethyl. When a (C₃₋ₘ)cycloalkyl-(C₁₋ₙ)alkyl- group is substituted, it is understood that substituents may be attached to either the cycloalkyl or the alkyl portion thereof or both, unless specified otherwise, such that the substitution would give rise to a chemically stable compound, such as are recognized by those skilled in the art.

The term “(C₅₋ₙ)cycloalkenyl” as used herein, wherein n is an integer, either alone or in combination with another radical, is intended to mean an unsaturated cyclic radical containing five to n carbon atoms. Examples include, but are not limited to, cyclopentenyl and cyclohexenyl. The term “(C₃₋ₙ)cycloalkenyl” as used herein, wherein n is an integer, either alone or in combination with another radical, is intended to mean an unsaturated cyclic radical containing three to n carbon atoms.

The term “aryl” as used herein, either alone or in combination with another radical, is intended to mean a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, indenyl, 1-naphthyl, 2-naphthyl, tetrahydronaphthyl and dihydronaphthyl.

The term “aryl-(C₁₋ₙ)alkyl-” as used herein, wherein n is an integer, either alone or in combination with another radical, is intended to mean an alkyl radical having 1 to n carbon atoms as defined above which is itself substituted with an aryl radical as defined above. Examples of aryl-(C₁₋ₙ)alkyl- include, but are not limited to, phenylmethyl (benzyl), 1-phenylethyl, 2-phenylethyl and phenylpropyl. When an aryl-(C₁₋ₙ)alkyl- group is substituted, it is understood that substituents may be attached to either the aryl or the alkyl portion thereof or both, unless specified otherwise, such that the substitution would give rise to a chemically stable compound, such as are recognized by those skilled in the art.

The term “Het” as used herein, either alone or in combination with another radical, is intended to mean a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S; wherein each N heteroatom may, independently and where possible, exist in an oxidized state such that it is further bonded to an oxygen atom to form an N-oxide group and wherein each S heteroatom may, independently and where possible, exist in an oxidized state such that it is further bonded to one or two oxygen atoms to form the groups SO or SO₂, unless specified otherwise. When a Het group is substituted, it is understood that substituents may be attached to any carbon atom or heteroatom thereof which would otherwise bear a hydrogen atom, unless specified otherwise, such that the substitution would give rise to a chemically stable compound, such as are recognized by those skilled in the art.

The term “Het-(C₁₋ₙ)alkyl-” as used herein and unless specified otherwise, wherein n is an integer, either alone or in combination with another radical, is intended to mean an alkyl radical having 1 to n carbon atoms as defined above which is itself substituted with a Het substituent as defined above. Examples of Het-(C₁₋ₙ)alkyl- include, but are not limited to, thienylmethyl, furylmethyl, piperidinylethyl, 2-pyridinylmethyl, 3-pyridinylmethyl, 4-pyridinylmethyl, quinolinylpropyl, and the like. When an Het-(C₁₋ₙ)alkyl- group is substituted, it is understood that substituents may be attached to either the Het or the alkyl portion thereof or both, unless specified otherwise, such that the substitution would give rise to a chemically stable compound, such as are recognized by those skilled in the art.

The term “heteroatom” as used herein is intended to mean O, S or N.

The term “heterocycle” as used herein and unless specified otherwise, either alone or in combination with another radical, is intended to mean a 3- to 7-membered saturated, unsaturated or aromatic heterocycle containing from 1 to 4 heteroatoms each independently selected from O, N and S; or a monovalent radical derived by removal of a hydrogen atom therefrom. Examples of such heterocycles include, but are not limited to, azetidine, pyrrolidine, tetrahydrofuran, tetrahydrothiophene, thiazolidine, oxazolidine, pyrrole, thiophene, furan, pyrazole, imidazole, isoxazole, oxazole, isothiazole, thiazole, triazole, tetrazole, piperidine, piperazine, azepine, diazepine, pyran, 1,4-dioxane, 4-morpholine, 4-thiomorpholine, pyridine, pyridine-N-oxide, pyridazine, pyrazine and pyrimidine, and saturated, unsaturated and aromatic derivatives thereof.

The term “heteropolycycle” as used herein and unless specified otherwise, either alone or in combination with another radical, is intended to mean a heterocycle as defined above fused to one or more other cycle, including a carbocycle, a heterocycle or any other cycle; or a monovalent radical derived by removal of a hydrogen atom therefrom. Examples of such heteropolycycles include, but are not limited to, indole, isoindole, tetrahydroindole, benzimidazole, benzothiophene, benzofuran, benzodioxole, benzothiazole, quinoline, isoquinoline, and naphthyridine.

The term “halo” as used herein is intended to mean a halogen substituent selected from fluoro, chloro, bromo and iodo.

The term “(C₁₋ₙ)haloalkyl” as used herein, wherein n is an integer, either alone or in combination with another radical, is intended to mean an alkyl radical having 1 to n carbon atoms as defined above wherein one or more hydrogen atoms are each replaced by a halo substituent. When two or more hydrogen atoms are replaced by halo substituents, the halo substituents may be the same or different. Examples of (C₁₋ₙ)haloalkyl include but are not limited to chloromethyl, chloroethyl, dichloroethyl, bromomethyl, bromoethyl, dibromoethyl, chlorobromoethyl, fluoromethyl, difluoromethyl, trifluoromethyl, fluoroethyl and difluoroethyl.

The terms “—O—(C₁₋ₙ)alkyl” or “(C₁₋ₙ)alkoxy” as used herein interchangeably, wherein n is an integer, either alone or in combination with another radical, are intended to mean an oxygen atom further bonded to an alkyl radical having 1 to n carbon atoms as defined above. Examples of —O—(C₁₋ₙ)alkyl include but are not limited to methoxy (CH₃O—), ethoxy (CH₃CH₂O—), propoxy (CH₃CH₂CH₂O—), 1-methylethoxy (iso-propoxy; (CH₃)₂CH—O—) and 1,1-dimethylethoxy (tert-butoxy; (CH₃)₃C—O—). When an —O—(C₁₋ₙ)alkyl radical is substituted, it is understood to be substituted on the (C₁₋ₙ)alkyl portion thereof, such that the substitution would give rise to a chemically stable compound, such as are recognized by those skilled in the art.

The terms “—S—(C₁₋ₙ)alkyl” or “(C₁₋ₙ)alkylthio” as used herein interchangeably, wherein n is an integer, either alone or in combination with another radical, are intended to mean an sulfur atom further bonded to an alkyl radical having 1 to n carbon atoms as defined above. Examples of —S—(C₁₋ₙ)alkyl include but are not limited to methylthio (CH₃S—), ethylthio (CH₃CH₂S—), propylthio (CH₃CH₂CH₂S—), 1-methylethylthio (isopropylthio; (CH₃)₂CH—S—) and 1,1-dimethylethylthio (tert-butylthio; (CH₃)₃C—S—). When —S—(C₁₋ₙ)alkyl radical, or an oxidized derivative thereof, such as an —SO—(C₁₋ₙ)alkyl radical or an —SO₂—(C₁₋ₙ)alkyl radical, is substituted, each is understood to be substituted on the (C₁₋ₙ)alkyl portion thereof, such that the substitution would give rise to a chemically stable compound, such as are recognized by those skilled in the art.

The term “oxo” as used herein is intended to mean an oxygen atom attached to a carbon atom as a substituent by a double bond (═O).

The term “thioxo” as used herein is intended to mean a sulfur atom attached to a carbon atom as a substituent by a double bond (═S).

The term “imino” as used herein is intended to mean a NH group attached to a carbon atom as a substituent by a double bond (═NH).

The term “COOH” as used herein is intended to mean a carboxyl group (—C(═O)—OH). It is well known to one skilled in the art that carboxyl groups may be substituted by functional group equivalents. Examples of such functional group equivalents contemplated in this invention include, but are not limited to, esters, amides, imides, boronic acids, phosphonic acids, phosphoric acids, tetrazoles, triazoles, N-acylsulfamides (RCONHSO₂NR₂), and N-acylsulfonamides (RCONHSO₂R).

The term “functional group equivalent” as used herein is intended to mean an atom or group that may replace another atom or group which has similar electronic, hybridization or bonding properties.

The term “protecting group” as used herein is intended to mean protecting groups that can be used during synthetic transformation, including but not limited to examples which are listed in Greene, “Protective Groups in Organic Chemistry”, John Wiley & Sons, New York (1981), and more recent editions thereof, herein incorporated by reference.

As used herein, the designation whereby a bond to a substituent R is drawn as emanating from the center of a ring, such as, for example,

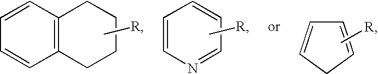

is intended to mean that the substituent R may be attached to any free position on the ring that would otherwise be substituted with a hydrogen atom, unless specified otherwise.

The following designation [symbol] is used in sub-formulas to indicate the bond which is connected to the rest of the molecule as defined.

The term “salt thereof” as used herein is intended to mean any acid and/or base addition salt of a compound according to the invention, including but not limited to a pharmaceutically acceptable salt thereof.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Berge et al., J. Pharm. Sci., 1977, 66, pp. 1-19, herein incorporated by reference.

The term “pharmaceutically-acceptable acid addition salt” as used herein is intended to mean those salts which retain the biological effectiveness and properties of the free bases and which are not biologically or otherwise undesirable, formed with inorganic acids or organic acids. Suitable inorganic acids include but are not limited to hydrochloric acid, hydrobromic acid, sulfuric acid, sulfamic acid, nitric acid, phosphoric acid and the like. Suitable organic acids include but are not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases or organic bases. Suitable inorganic bases include but are not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “mammal” as used herein is intended to encompass humans, as well as non-human mammals which are susceptible to infection by hepatitis C virus. Non-human mammals include but are not limited to domestic animals, such as cows, pigs, horses, dogs, cats, rabbits, rats and mice, and non-domestic animals.

The term “treatment” as used herein is intended to mean the administration of a compound or composition according to the present invention to alleviate or eliminate symptoms of the hepatitis C disease and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectable levels in the blood.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal.

### Preferred Embodiments

In the following preferred embodiments, groups and substituents of the compounds according to this invention are described in detail.

A particular aspect of the invention provides compounds of formula (I):

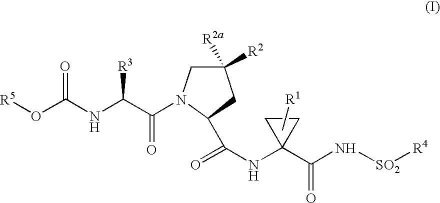

wherein, particularly:
a) the R¹ substituent is selected from:

a-1) R¹ is (C₁₋₄)alkyl or (C₂₋₄)alkenyl;

a-2) R¹ is (C₁₋₃) alkyl or (C₂₋₄) alkenyl;

a-3) R¹ is (C₂₋₃) alkenyl; or

a-4) R¹ is CH═CH₂ (vinyl).

Any and each individual definition of R¹ as set out herein may be combined with any and each individual definition of R², R^{2a}, R²⁰, R²¹, R³, R⁴ and R⁵ as set out herein.

b) the R² substituent is selected from:

b-1): R² is —OMe; —OEt; —OPr; —OButyl; —OPentyl or —OHexyl;

b-2): R² is ±OMe; —OEt; —O-nPr; or —O-iPr;

b-3) R² is —OMe or —OEt; or

b-4) R² is OMe.

Any and each individual definition of R² as set out herein may be combined with any and each individual definition of R¹, R^{2a}, R²⁰, R²¹, R³, R⁴ and R⁵ as set out herein.

c) the R^{2a} substituent is selected from:

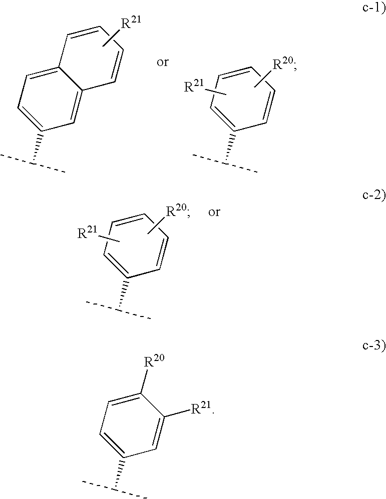

Any and each individual definition of R^{ea} as set out herein may be combined with any and each individual definition of R¹, R², R²⁰, R²¹, R³, R⁴ and R⁵ as set out herein.
c′) wherein R²⁰ may be selected from:
c′-1) phenyl and Het, each optionally substituted with one or more substituents each independently selected from halogen, (C₁₋₆)alkyl, (C₁₋₆)haloalkyl, —O(C₁₋₆)alkyl, —S(C₁₋₆)alkyl, —OH, —SH, —NH₂, —NH(C₁₋₆)alkyl, —N((C₁₋₆)alkyl)₂, and —NHC(═O)(C₁₋₆)alkyl;
c′-2) phenyl and Het, each optionally substituted with one or more substituents each independently selected from halogen, (C₁₋₄)alkyl, (C₁₋₄haloalkyl, —O(C₁₋₄)alkyl, —S(C₁₋₄)alkyl, —OH, —SH, —NH₂, —NH(C₁₋₃)alkyl, —N((C₁₋₃)alkyl)₂, and —NHC(═O)(C₁₋₃)alkyl;
c′-3) phenyl and Het, each optionally substituted with one or two substituents each independently selected from Cl, F, Br, Me, Et, MeO, EtO, MeS, and EtS; wherein said Het is selected from:

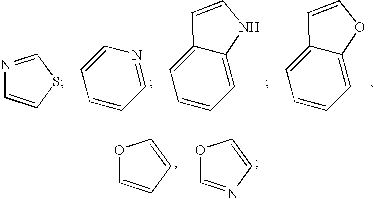

Any and each individual definition of R²⁰ as set out herein may be combined with any and each individual definition of R², R^{2a}, R¹, R²¹, R³, R⁴ and R⁵ as set out herein.
c″) and R²¹ may be selected from:
c″-1) is one to four substituents each independently selected from H, halogen, (C₁₋₆)alkyl and —O(C₁₋₆)alkyl;
c″-2) is one to three substituents each independently selected from H, halogen, and (C₁₋₃)alkyl;
c″-3) is a substituent independently selected from: H, F or Me.

Any and each individual definition of R²¹ as set out herein may be combined with any and each individual definition of R², R^{2a}, R²⁰, R¹, R³, R⁴ and R⁵ as set out herein.

### d) the R³ Substituent is Selected From:

d-1) R³ is (C₁₋₈)alkyl or (C₃₋₇)cycloalkyl, each optionally substituted with one substituent selected from: (C₁₋₆)alkyl, halogen, —SR³⁰, wherein R³⁰ is H or (C₁₋₆)alkyl;
d-2) R³ is (C₁₋₈)alkyl optionally substituted with —S(C₁₋₆)alkyl; or (C₃₋₇)cycloalkyl optionally substituted with (C₁₋₆)alkyl;
d-3) R³ is (C₁₋₄)alkyl; or (C₆)cycloalkyl; or
d-4) R³ is tert-butyl.

Any and each individual definition of R³ as set out herein may be combined with any and each individual definition of R², R^{2a}, R²⁰, R²¹, R¹, R⁴ and R⁵ as set out herein.

### e) the R⁴ Substituent is Selected From:

e-1) R⁴ is (C₃₋₇)cycloalkyl; said (C₃₋₇)cycloalkyl being optionally substituted with (C₁₋₆)alkyl; or R⁴ is —NHR^{N1}, wherein R^{N1} is H or (C₁₋₆)alkyl;
e-2) R⁴ is (C₃₋₆)cycloalkyl optionally substituted with (C₁₋₆)alkyl;
e-3) R⁴ is (C₃₋₄)cycloalkyl optionally substituted with methyl; or
e-4) R⁴ is cyclopropyl.

Any and each individual definition of R⁴ as set out herein may be combined with any and each individual definition of R², R^{2a}, R²⁰, R²¹, R³, R¹ and R⁵ as set out herein.

### f) the R⁵ Substituent is Selected From:

f-1) R⁵ is (C₁₋₁₀)alkyl optionally substituted with one or more halogen; or (C₃₋₇)cycloalkyl optionally substituted with one or more (C₁₋₆)alkyl;
f-2) R⁵ is (C₁₋₆)alkyl optionally substituted with fluoro; or (C₃₋₅)cycloalkyl optionally substituted with methyl;
f-3) R⁵ is (C₃₋₄)alkyl; or (C₃₋₅)cycloalkyl; or
f-4) R⁵ is tert-butyl or cyclopentyl.

Any and each individual definition of R⁵ as set out herein may be combined with any and each individual definition of R², R^{2a}, R²⁰, R²¹, R³, R⁴ and R¹ as set out herein.

Examples of preferred subgeneric embodiments of the present invention are set forth in the following table, wherein each substituent group of each embodiment is defined according to the definitions set forth above:

| Embodiment | R¹ | R² | R^{2a} | R²⁰ | R²¹ | R³ | R⁴ | R⁵ |
|---|---|---|---|---|---|---|---|---|
| A-1 | a-1 | b-1 | c-1 | c′-1 | c″-1 | d-1 | e-1 | f-1 |
| A-2 | a-2 | b-2 | c-2 | c′-2 | c″-2 | d-2 | e-2 | f-2 |
| A-3 | a-3 | b-3 | c-3 | c′-3 | c″-3 | d-3 | e-3 | f-3 |
| A-4 | a-4 | b-4 | c-3 | c′-3 | c″-3 | d-4 | e-4 | f-4 |
| B-1 | a-3 | b-3 | c-2 | c′-2 | c″-2 | d-3 | e-3 | f-3 |
| B-2 | a-3 | b-4 | c-2 | c′-2 | c″-2 | d-3 | e-3 | f-3 |
| B-3 | a-3 | b-3 | c-2 | c′-2 | c″-2 | d-4 | e-3 | f-3 |
| B-4 | a-3 | b-3 | c-2 | c′-2 | c″-2 | d-3 | e-4 | f-3 |
| B-5 | a-3 | b-3 | c-2 | c′-2 | c″-2 | d-3 | e-3 | f-4 |
| B-6 | a-4 | b-4 | c-2 | c′-2 | c″-2 | d-3 | e-3 | f-3 |
| B-7 | a-4 | b-3 | c-2 | c′-2 | c″-2 | d-4 | e-3 | f-4 |
| B-8 | a-4 | b-3 | c-2 | c′-2 | c″-2 | d-3 | e-4 | f-4 |
| B-9 | a-4 | b-3 | c-2 | c′-2 | c″-2 | d-3 | e-3 | f-4 |
| B-10 | a-3 | b-4 | c-2 | c′-2 | c″-2 | d-4 | e-3 | f-3 |
| B-11 | a-3 | b-4 | c-2 | c′-2 | c″-2 | d-3 | e-4 | f-3 |
| B-12 | a-3 | b-4 | c-2 | c′-2 | c″-2 | d-3 | e-3 | f-4 |
| B-13 | a-3 | b-3 | c-2 | c′-2 | c″-2 | d-4 | e-4 | f-4 |
| B-14 | a-3 | b-3 | c-2 | c′-2 | c″-2 | d-3 | e-4 | f-4 |
| B-15 | a-4 | b-4 | c-2 | c′-2 | c″-2 | d-4 | e-3 | f-3 |
| B-16 | a-4 | b-3 | c-2 | c′-2 | c″-2 | d-4 | e-4 | f-3 |
| B-17 | a-4 | b-3 | c-2 | c′-2 | c″-2 | d-3 | e-4 | f-4 |
| B-18 | a-4 | b-4 | c-2 | c′-2 | c″-2 | d-4 | e-4 | f-3 |
| B-19 | a-4 | b-3 | c-2 | c′-2 | c″-2 | d-4 | e-4 | f-4 |
| B-20 | a-3 | b-4 | c-2 | c′-2 | c″-2 | d-4 | e-4 | f-4 |
| C-1 | a-3 | b-3 | c-3 | c′-3 | c″-3 | d-3 | e-3 | f-3 |
| C-2 | a-3 | b-4 | c-3 | c′-3 | c″-3 | d-3 | e-3 | f-3 |
| C-3 | a-3 | b-3 | c-3 | c′-3 | c″-3 | d-4 | e-3 | f-3 |
| C-4 | a-3 | b-3 | c-3 | c′-3 | c″-3 | d-3 | e-4 | f-3 |
| C-5 | a-3 | b-3 | c-3 | c′-3 | c″-3 | d-3 | e-3 | f-4 |
| C-6 | a-4 | b-4 | c-3 | c′-3 | c″-3 | d-3 | e-3 | f-3 |
| C-7 | a-4 | b-3 | c-3 | c′-3 | c″-3 | d-4 | e-3 | f-3 |
| C-8 | a-4 | b-3 | c-3 | c′-3 | c″-3 | d-3 | e-4 | f-3 |
| C-9 | a-4 | b-3 | c-3 | c′-3 | c″-3 | d-3 | e-3 | f-4 |
| C-10 | a-3 | b-4 | c-3 | c′-3 | c″-3 | d-4 | e-3 | f-3 |
| C-11 | a-3 | b-4 | c-3 | c′-3 | c″-3 | d-3 | e-4 | f-3 |
| C-12 | a-3 | b-4 | c-3 | c′-3 | c″-3 | d-3 | e-3 | f-4 |
| C-13 | a-3 | b-3 | c-3 | c′-3 | c″-3 | d-4 | e-4 | f-3 |
| C-14 | a-3 | b-3 | c-3 | c′-3 | c″-3 | d-3 | e-4 | f-4 |
| C-15 | a-4 | b-4 | c-3 | c′-3 | c″-3 | d-4 | e-3 | f-3 |
| C-16 | a-4 | b-3 | c-3 | c′-3 | c″-3 | d-4 | e-4 | f-3 |
| C-17 | a-4 | b-3 | c-3 | c′-3 | c″-3 | d-3 | e-4 | f-4 |
| C-18 | a-4 | b-4 | c-3 | c′-3 | c″-3 | d-4 | e-4 | f-3 |
| C-19 | a-4 | b-3 | c-3 | c′-3 | c″-3 | d-4 | e-4 | f-4 |
| C-20 | a-3 | b-4 | c-3 | c′-3 | c″-3 | d-4 | e-4 | f-4 |
| D-1 | a-4 | b-4 | c-2 | c′-2 | c″-2 | d-4 | e-4 | f-4 |
| D-2 | a-4 | b-3 | c-2 | c′-2 | c″-2 | d-4 | e-3 | f-4 |
| D-3 | a-4 | b-4 | c-2 | c′-2 | c″-2 | d-3 | e-4 | f-4 |
| D-4 | a-4 | b-4 | c-2 | c′-2 | c″-2 | d-4 | e-3 | f-4 |
| D-4 | a-4 | b-4 | c-2 | c′-2 | c″-2 | d-4 | e-4 | f-3 |
| D-6 | a-3 | b-3 | c-2 | c′-2 | c″-2 | d-4 | e-4 | f-4 |
| D-7 | a-3 | b-4 | c-2 | c′-2 | c″-2 | d-3 | e-4 | f-4 |
| D-8 | a-3 | b-4 | c-2 | c′-2 | c″-2 | d-4 | e-3 | f-4 |
| D-9 | a-3 | b-4 | c-2 | c′-2 | c″-2 | d-4 | e-4 | f-3 |
| D-10 | a-4 | b-3 | c-2 | c′-2 | c″-2 | d-3 | e-4 | f-4 |
| D-11 | a-4 | b-3 | c-2 | c′-2 | c″-2 | d-4 | e-3 | f-4 |
| D-12 | a-4 | b-3 | c-2 | c′-2 | c″-2 | d-4 | e-4 | f-3 |
| D-13 | a-4 | b-4 | c-2 | c′-2 | c″-2 | d-3 | e-3 | f-4 |
| D-14 | a-4 | b-4 | c-2 | c′-2 | c″-2 | d-4 | e-3 | f-3 |
| D-15 | a-3 | b-3 | c-2 | c′-2 | c″-2 | d-3 | e-4 | f-4 |
| D-16 | a-3 | b-4 | c-2 | c′-2 | c″-2 | d-3 | e-3 | f-4 |
| D-17 | a-3 | b-4 | c-2 | c′-2 | c″-2 | d-4 | e-3 | f-3 |
| D-18 | a-3 | b-3 | c-2 | c′-2 | c″-2 | d-3 | e-3 | f-4 |
| D-19 | a-3 | b-4 | c-2 | c′-2 | c″-2 | d-3 | e-3 | f-3 |
| D-20 | a-4 | b-3 | c-2 | c′-2 | c″-2 | d-3 | e-3 | f-3 |
| E-1 | a-4 | b-4 | c-3 | c′-3 | c″-3 | d-4 | e-4 | f-4 |
| E-2 | a-4 | b-3 | c-3 | c′-3 | c″-3 | d-4 | e-3 | f-4 |
| E-3 | a-4 | b-4 | c-3 | c′-3 | c″-3 | d-3 | e-4 | f-4 |
| E-4 | a-4 | b-4 | c-3 | c′-3 | c″-3 | d-4 | e-3 | f-4 |
| E-5 | a-4 | b-4 | c-3 | c′-3 | c″-3 | d-4 | e-4 | f-3 |
| E-6 | a-3 | b-3 | c-3 | c′-3 | c″-3 | d-4 | e-4 | f-4 |
| E-7 | a-3 | b-4 | c-3 | c′-3 | c″-3 | d-3 | e-4 | f-4 |
| E-8 | a-3 | b-4 | c-3 | c′-3 | c″-3 | d-4 | e-3 | f-4 |
| E-9 | a-3 | b-4 | c-3 | c′-3 | c″-3 | d-4 | e-4 | f-3 |
| E-10 | a-4 | b-3 | c-3 | c′-3 | c″-3 | d-3 | e-4 | f-4 |
| E-11 | a-4 | b-3 | c-3 | c′-3 | c″-3 | d-4 | e-3 | f-4 |
| E-12 | a-4 | b-3 | c-3 | c′-3 | c″-3 | d-4 | e-4 | f-3 |
| E-13 | a-4 | b-4 | c-3 | c′-3 | c″-3 | d-3 | e-3 | f-4 |
| E-14 | a-4 | b-4 | c-3 | c′-3 | c″-3 | d-4 | e-3 | f-3 |
| E-15 | a-3 | b-3 | c-3 | c′-3 | c″-3 | d-3 | e-4 | f-4 |
| E-16 | a-3 | b-4 | c-3 | c′-3 | c″-3 | d-3 | e-3 | f-4 |
| E-17 | a-3 | b-4 | c-3 | c′-3 | c″-3 | d-4 | e-3 | f-3 |
| E-18 | a-3 | b-3 | c-3 | c′-3 | c″-3 | d-3 | e-3 | f-4 |
| E-19 | a-3 | b-4 | c-3 | c′-3 | c″-3 | d-3 | e-3 | f-3 |
| E-20 | a-4 | b-3 | c-3 | c′-3 | c″-3 | d-3 | e-3 | f-3 |

Examples of most preferred compounds according to this invention are each single compound listed in the following Tables 1 and 2.

In general, all tautomeric and isomeric forms and mixtures thereof, for example, individual geometric isomers, stereoisomers, enantiomers, diastereomers, racemates, racemic or non-racemic mixtures of stereoisomers, mixtures of diastereomers, or mixtures of any of the foregoing forms of a chemical structure or compound is intended, unless the specific stereochemistry or isomeric form is specifically indicated in the compound name or structure.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the present invention from this disclosure and the knowledge in the art.

Preparation of pure stereoisomers, e.g. enantiomers and diastereomers, or mixtures of desired enantiomeric excess (ee) or enantiomeric purity, are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000, herein incorporated by reference. Furthermore, there are equally well-known methods for the quantitation of enantiomeric excess or purity, for example, GC, HPLC, CE, or NMR, and assignment of absolute configuration and conformation, for example, CD ORD, X-ray crystallography, or NMR.

A compound according to the present invention may also be used as a laboratory reagent or a research reagent. For example, a compound of the present invention may be used as positive control to validate assays, including but not limited to surrogate cell-based assays and in vitro or in vivo viral replication assays.

Furthermore, a compound according to the present invention may be used to treat or prevent viral contamination of materials and therefore reduce the risk of viral infection of laboratory or medical personnel or patients who come in contact with such materials (e.g. blood, tissue, surgical instruments and garments, laboratory instruments and garments, and blood collection apparatuses and materials).

### Pharmaceutical Composition

Compounds of the present invention may be administered to a mammal in need of treatment for hepatitis C viral infection as a pharmaceutical composition comprising a therapeutically effective amount of a compound according to the invention or a pharmaceutically acceptable salt thereof; and one or more conventional non-toxic pharmaceutically-acceptable carriers, adjuvants or vehicles. The specific formulation of the composition is determined by the solubility and chemical nature of the compound, the chosen route of administration and standard pharmaceutical practice. The pharmaceutical composition according to the present invention may be administered orally or systemically.

When one enantiomer of a chiral active ingredient has a different biological activity than the other, it is contemplated that the pharmaceutical composition according to the invention may comprise a racemic mixture of the active ingredient, a mixture enriched in one enantiomer of the active ingredient or a pure enantiomer of the active ingredient. The mixture enriched in one enantiomer of the active ingredient is contemplated to contain from about 50% to about 100% of one enantiomer of the active ingredient and from about 0% to about 50% of the other enantiomer of the active ingredient. Preferably, when the composition comprises a mixture enriched in one enantiomer of the active ingredient or a pure enantiomer of the active ingredient, the composition comprises from about 50% to about 100% of, or only, the more physiologically active enantiomer and/or the less toxic enantiomer. It is well known that one enantiomer of an active ingredient may be the more physiologically active for one therapeutic indication while the other enantiomer of the active ingredient may be the more physiologically active for a different therapeutic indication; therefore the preferred enantiomeric makeup of the pharmaceutical composition may differ for use of the composition in treating different therapeutic indications.

For oral administration, the compound, or a pharmaceutically acceptable salt thereof, can be formulated in any orally acceptable dosage form including but not limited to aqueous suspensions and solutions, capsules or tablets. For systemic administration, including but not limited to administration by subcutaneous, intracutaneous, intravenous, intramuscular, intra-articular, intrasynovial, intrasternal, intrathecal, and intralesional injection or infusion techniques, it is preferred to use a solution of the compound, or a pharmaceutically acceptable salt thereof, in a pharmaceutically acceptable sterile aqueous vehicle.

Pharmaceutically acceptable carriers, adjuvants, vehicles, excipients and additives as well as methods of formulating pharmaceutical compositions for various modes of administration are well-known to those of skill in the art and are described in pharmaceutical texts such as Remington: The Science and Practice of Pharmacy, 21st Edition, Lippincott Williams & Wilkins, 2005; and L. V. Allen, N. G. Popovish and H. C. Ansel, Pharmaceutical Dosage Forms and Drug Delivery Systems, 8th ed., Lippincott Williams & Wilkins, 2004, herein incorporated by reference.

The dosage administered will vary depending upon known factors, including but not limited to the activity and pharmacodynamic characteristics of the specific compound employed and its mode, time and route of administration; the age, diet, gender, body weight and general health status of the recipient; the nature and extent of the symptoms; the severity and course of the infection; the kind of concurrent treatment; the frequency of treatment; the effect desired; and the judgment of the treating physician. In general, the compound is most desirably administered at a dosage level that will generally afford antivirally effective results without causing any harmful or deleterious side effects.

A daily dosage of active ingredient can be expected to be about 0.01 to about 100 milligrams per kilogram of body weight, with the preferred dose being about 0.1 to about 50 mg/kg. Typically, the pharmaceutical composition of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

### Combination Therapy

Combination therapy is contemplated wherein a compound according to the invention, or a pharmaceutically acceptable salt thereof, is co-administered with at least one additional antiviral agent. The additional agents may be combined with compounds of this invention to create a single dosage form. Alternatively these additional agents may be separately administered, concurrently or sequentially, as part of a multiple dosage form.

When the pharmaceutical composition of this invention comprises a combination of a compound according to the invention, or a pharmaceutically acceptable salt thereof, and one or more additional antiviral agent, both the compound and the additional agent should be present at dosage levels of between about 10 to 100%, and more preferably between about 10 and 80% of the dosage normally administered in a monotherapy regimen. In the case of a synergistic interaction between the compound of the invention and the additional antiviral agent or agents, the dosage of any or all of the active agents in the combination may be reduced compared to the dosage normally administered in a monotherapy regimen.

Antiviral agents contemplated for use in such combination therapy include agents (compounds or biologicals) that are effective to inhibit the formation and/or replication of a virus in a mammal, including but not limited to agents that interfere with either host or viral mechanisms necessary for the formation and/or replication of a virus in a mammal. Such agents can be selected from another anti-HCV agent; an HIV inhibitor; an HAV inhibitor; and an HBV inhibitor.

Other anti-HCV agents include those agents that are effective for diminishing or preventing the progression of hepatitis C related symptoms or disease. Such agents include but are not limited to immunomodulatory agents, inhibitors of HCV NS3 protease, HCV polymerase, HCV helicase, HCV NS4B protein, HCV entry, HCV assembly, HCV NS5A protein, HCV NS5B protein, inhibitors of another target in the HCV life cycle and other anti-HCV agents, including but not limited to nucleoside analogs for the treatment of HCV infection, ribavirin, amantadine, levovirin and viramidine.

Immunomodulatory agents include those agents (compounds or biologicals) that are effective to enhance or potentiate the immune system response in a mammal. Immunomodulatory agents include, but are not limited to, inosine monophosphate dehydrogenase inhibitors such as VX-497 (merimepodib, Vertex Pharmaceuticals), class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, α-, β-, δ-, ω-, and τ-interferons, while examples of class II interferons include, but are not limited to, γ-interferons. In one preferred aspect, the other anti-HCV agent is an interferon. Preferably, the interferon is selected from the group consisting of interferon alpha 2B, pegylated interferon alpha, consensus interferon, interferon alpha 2A and lymphoblastoid interferon. In one preferred aspect, the composition comprises a compound of the invention, an interferon and ribavirin.

Inhibitors of HCV NS3 protease include agents (compounds or biologicals) that are effective to inhibit the function of HCV NS3 protease in a mammal. Inhibitors of HCV NS3 protease include, for example, those compounds described in WO 99/07733, WO 99/07734, WO 00/09558, WO 00/09543, WO 00/59929, WO 03/064416, WO 03/064455, WO 03/064456, WO 2004/030670, WO 2004/037855, WO 2004/039833, WO 2004/101602, WO 2004/101605, WO 2004/103996, WO 2005/028501, WO 2005/070955, WO 2006/000085, WO 2006/007700, WO 2006/007708, WO 2007/009227 (all by Boehringer Ingelheim), WO 02/060926, WO 03/053349, WO 03/099274, WO 03/099316, WO 2004/032827, WO 2004/043339, WO 2004/094452, WO 2005/046712, WO 2005/051410, WO 2005/054430 (all by BMS), WO 2004/072243, WO 2004/093798, WO 2004/113365, WO 2005/010029 (all by Enanta), WO 2005/037214 (Intermune), WO 01/77113, WO 01/81325, WO 02/08187, WO 02/08198, WO 02/08244, WO 02/08256, WO 02/48172, WO 03/062228, WO 03/062265, WO 2005/021584, WO 2005/030796, WO 2005/058821, WO 2005/051980, WO 2005/085197, WO 2005/085242, WO 2005/085275, WO 2005/087721, WO 2005/087725, WO 2005/087730, WO 2005/087731, WO 2005/107745 and WO 2005/113581 (all by Schering), WO 2006/119061, WO 2007/016441, WO 2007/015855, WO 2007/015787 (all by Merck), WO 2006/043145 (Pfizer), all of which are herein incorporated by reference; and the candidates VX-950, SCH-503034, ITMN-191, TMC 435350, and MK7009.

Inhibitors of HCV polymerase include agents (compounds or biologicals) that are effective to inhibit the function of an HCV polymerase. Such inhibitors include, but are not limited to, non-nucleoside and nucleoside inhibitors of NS4A, NS5A, NS5B polymerase. Examples of inhibitors of HCV polymerase include but are not limited to those compounds described in: WO 02/04425, WO 03/007945, WO 03/010140, WO 03/010141, WO 2004/064925, WO 2004/065367, WO 2005/080388, WO 2006/007693, WO 2007/019674, WO 2007/087717(all by Boehringer Ingelheim), WO 01/47883 (Japan Tobacco), WO 03/000254 (Japan Tobacco), WO 2007/033032, WO 2007/033175, WO 2006/020082, US 2005/0119318, WO 2005/034850, WO 03/026587, WO 2007/092000, WO 2007/143521, WO 2007/136982, WO 2007/140254, WO 2007/140200, WO 2007/092888 (all by BMS), WO 2007/095269, WO 2007/054741, WO 03/062211, WO 99/64442, WO 00/06529, WO 2004/110442, WO 2005/034941, WO 2006/119975, WO 2006/046030, WO 2006/046039, WO 2005/023819, WO 02/06246, WO 2007/065883, WO 2007/129119, WO 2007/029029, WO 2006/029912, WO 2006/027628, WO 2007/028789, WO 2006/008556, WO 2004/087714 (all by IRBM), WO 2005/012288 (Genelabs), WO 2005/014543 (Japan Tobacco), WO 2005/049622 (Japan Tobacco), and WO 2005/121132 (Shionogi), WO 2005/080399 (Japan Tobacco), WO 2006/052013 (Japan Tobacco), WO 2006/119646 (Virochem Pharma), WO 2007/039146 (SmithKline Beecham), WO 2005/021568 (Biota), WO 2006/094347 (Biota), WO 2006/093801, WO 2005/019191, WO 2004/041818, US 2004/0167123, US 2005/0107364 (all by Abbott Laboratories), WO 2007/034127 (Arrow Therapeutics Limited) (all of which are herein incorporated by reference) and the candidates HCV 796 (ViroPharma/Wyeth), R-1626, R-1656 and R-7128 (Roche), NM 283 (Idenix/Novartis), VCH-759 (Virochem), GS9190 (Gilead), MK-608 (Merck) and PF868554 (Pfizer).

The term “inhibitor of another target in the HCV life cycle” as used herein means an agent (compound or biological) that is effective to inhibit the formation and/or replication of HCV in a mammal other than by inhibiting the function HCV polymerase. This includes agents that interfere with either host or HCV viral targets necessary for the HCV life cycle or agents which specifically inhibit in HCV cell culture assays through an undefined or incompletely defined mechanism. Inhibitors of another target in the HCV life cycle include, for example, agents that inhibit viral targets such as Core, E1, E2, p7, NS2/3 protease, NS3 helicase, internal ribosome entry site (IRES), HCV entry and HCV assembly or host targets such as cyclophilin B, phosphatidylinositol 4-kinase IIIα, CD81, SR-B1, Claudin 1, VAP-A, VAP-B. Specific examples of inhibitors of another target in the HCV life cycle include ISIS-14803 (ISIS Pharmaceuticals), GS9190 (Gilead), GS9132 (Gilead), A-831 (AstraZeneca), NM-811 (Novartis), and DEBIO-025 (Debio Pharma).

It can occur that a patient may be co-infected with hepatitis C virus and one or more other viruses, including but not limited to human immunodeficiency virus (HIV), hepatitis A virus (HAV) and hepatitis B virus (HBV). Thus also contemplated is combination therapy to treat such co-infections by co-administering a compound according to the present invention with at least one of an HIV inhibitor, an HAV inhibitor and an HBV inhibitor.

HIV inhibitors include agents (compounds or biologicals) that are effective to inhibit the formation and/or replication of HIV. This includes but is not limited to agents that interfere with either host or viral mechanisms necessary for the formation and/or replication of HIV in a mammal. HIV inhibitors include, but are not limited to:
NRTIs (nucleoside or nucleotide reverse transcriptase inhibitors) including but not limited to zidovudine (AZT), didanosine (ddI), zalcitabine (ddC), stavudine (d4T), lamivudine (3TC), emtricitabine, abacavir succinate, elvucitabine, adefovir dipivoxil, lobucavir (BMS-180194) Iodenosine (FddA) and tenofovir including tenofovir disoproxil and tenofovir disoproxil fumarate salt, COMBIVIR™ (contains 3TC and AZT), TRIZIVIR™ (contains abacavir, 3TC and AZT), TRUVADA™ (contains tenofovir and emtricitabine), EPZICOM™ (contains abacavir and 3TC);
NNRTIs (non-nucleoside reverse transcriptase inhibitors) including but not limited to nevirapine, delaviradine, efavirenz, etravirine and rilpivirine;
protease inhibitors including but not limited to ritonavir, tipranavir, saquinavir, nelfinavir, indinavir, amprenavir, fosamprenavir, atazanavir, lopinavir, darunavir, lasinavir, brecanavir, VX-385 and TMC-114;
entry inhibitors including but not limited to
   CCR5 antagonists (including but not limited to maraviroc, vicriviroc, INCB9471 and TAK-652),
   CXCR4 antagonists (including but not limited to AMD-11070),
   fusion inhibitors (including but not limited to enfuvirtide (T-20), TR1-1144 and TR1-999) and
   others (including but not limited to BMS-488043);
integrase inhibitors (including but not limited to raltegravir (MK-0518), BMS-707035 and elvitegravir (GS 9137));
TAT inhibitors;
maturation inhibitors (including but not limited to berivimat (PA-457));
   immunomodulating agents (including but not limited to levamisole); and
other antiviral agents including hydroxyurea, ribavirin, IL-2, IL-12 and pensafuside.

HAV inhibitors include agents (compounds or biologicals) that are effective to inhibit the formation and/or replication of HAV. This includes but is not limited to agents that interfere with either host or viral mechanisms necessary for the formation and/or replication of HAV in a mammal. HAV inhibitors include but are not limited to Hepatitis A vaccines.

HBV inhibitors include agents (compounds or biologicals) that are effective to inhibit the formation and/or replication of HBV in a mammal. This includes but is not limited to agents that interfere with either host or viral mechanisms necessary for the formation and/or replication of HBV in a mammal. HBV inhibitors include, but are not limited to, agents that inhibit the HBV viral DNA polymerase and HBV vaccines.

Therefore, according to one embodiment, the pharmaceutical composition of this invention additionally comprises a therapeutically effective amount of one or more antiviral agents.

A further embodiment provides the pharmaceutical composition of this invention wherein the one or more antiviral agent comprises at least one other anti-HCV agent.

According to a more specific embodiment of the pharmaceutical composition of this invention, the at least one other anti-HCV agent comprises at least one immunomodulatory agent.

According to another more specific embodiment of the pharmaceutical composition of this invention, the at least one other anti-HCV agent comprises at least one inhibitor of HCV polymerase.

According to yet another more specific embodiment of the pharmaceutical composition of this invention, the at least one other anti-HCV agent comprises at least one other inhibitor of HCV NS3 protease.

According to still another more specific embodiment of the pharmaceutical composition of this invention, the at least one other anti-HCV agent comprises at least one inhibitor of another target in the HCV life cycle.

### Methodology and Synthesis

The compounds of the present invention are synthesized according to a general process wherein the P3, P2, P1, and P1′ fragments can be linked by well known peptide coupling techniques. The P3, P2, P1, and P1′ fragments may be linked together in any order as long as the final compound corresponds to compounds of formula (I), wherein R¹, R², R²⁰, R²¹, R³, R⁴, and R⁵ are as defined herein. For example, P3 can be linked to P2-P1-P1′, or P1-P1′ linked to P3-P2. This process is illustrated in Scheme I (wherein CPG is a carboxyl protecting group and APG is an amino protecting group).

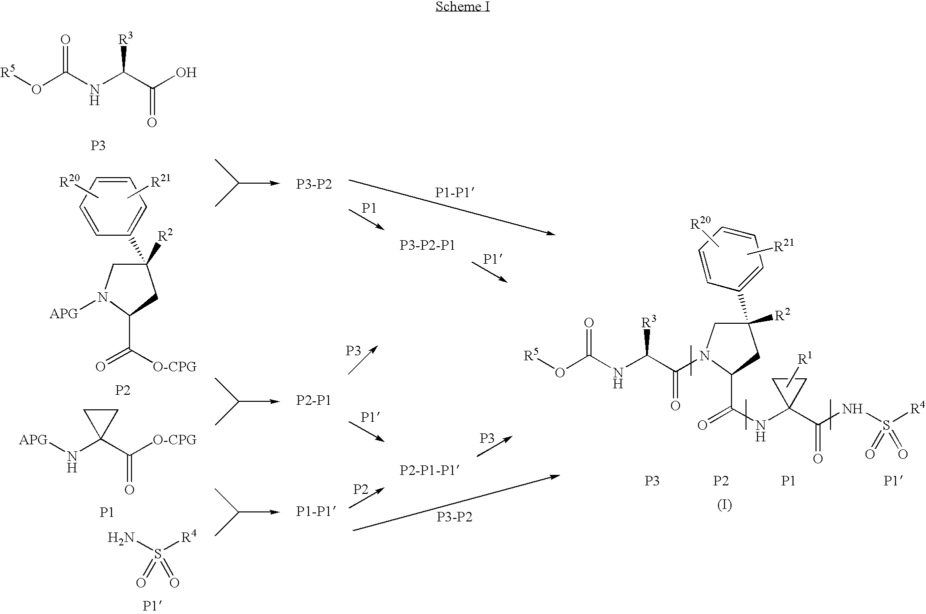

The P2 fragment may be formed by attaching the R² and substituted phenyl moieties to the proline fragment using methodology described in the examples below. This attachment may take place at any stage in this synthetic scheme, i.e., when P2 is an isolated fragment or when it has already been coupled to P3 and/or P1 or P1-P1′. In cases where the R² and substituted phenyl moieties are to be added at an intermediate stage after coupling to the P3 and/or P1 or P1-P1′ fragments, the P2 fragment shown above is replaced with a suitable precursor fragment for the purposes of this scheme.

Generally, peptides are elongated by deprotecting the α-amino group of the N-terminal residue and coupling the unprotected carboxyl group of the next suitably N-protected amino acid through a peptide linkage using well known methods. This deprotection and coupling procedure is repeated until the desired sequence is obtained. This coupling can be performed with the constituent amino acid fragments in stepwise fashion or by solid phase peptide synthesis according to the method originally described in Merrifield, J. Am. Chem. Soc., (1963), 85, 2149-2154, herein incorporated by reference.

Coupling between two amino acids, an amino acid and a peptide, or two peptide fragments can be carried out using standard coupling procedures such as the azide method, mixed carbonic-carboxylic acid anhydride (isobutyl chloroformate) method, carbodiimide (dicyclohexylcarbodiimide, diisopropylcarbodiimide, or water-soluble carbodiimide) method, active ester (p-nitrophenyl ester, N-hydroxysuccinic imido ester) method, Woodward reagent K-method, carbonyldiimidazole method, phosphorus reagents or oxidation-reduction methods. Some of these methods (especially the carbodiimide method) can be enhanced by adding 1-hydroxybenzotriazole. These coupling reactions can be performed in either solution (liquid phase) or solid phase.

More explicitly, the coupling step involves the dehydrative coupling of a free carboxyl of one reactant with the free amino group of the other reactant in the presence of a coupling agent to form a linking amide bond. Descriptions of such coupling agents are found in general textbooks on peptide chemistry, for example, M. Bodanszky, “Peptide Chemistry”, 2nd rev ed., Springer-Verlag, Berlin, Germany, (1993), herein incorporated by reference. Examples of suitable coupling agents are N,N′-dicyclohexylcarbodiimide, 1-hydroxybenzotriazole in the presence of N,N′-dicyclohexylcarbodiimide or N-ethyl-N′-[(3-dimethylamino)propyl]carbodiimide. A practical and useful coupling agent is the commercially available (benzotriazol-1-yloxy)tris-(dimethylamino)phosphonium hexafluorophosphate, either by itself or in the presence of 1-hydroxybenzotriazole. Another practical and useful coupling agent is commercially available 2-(1H-benzotriazol-1-yl)-N,N,N′,N′-tetramethyluronium tetrafluoroborate. Still another practical and useful coupling agent is commercially available O-(7-azabenzotriazol-1-yl)-N,N,N′,N′-tetramethyluronium hexafluorophosphate.

The coupling reaction is conducted in an inert solvent, e.g. dichloromethane, acetonitrile or dimethylformamide. An excess of a tertiary amine, e.g. diisopropylethylamine, N-methylmorpholine or N-methylpyrrolidine, is added to maintain the reaction mixture at a pH of about 8. The reaction temperature usually ranges between 0° C. and 50° C. and the reaction time usually ranges between 15 min and 24 h.

When a solid phase synthetic approach is employed, the C-terminal carboxylic acid is attached to an insoluble carrier (usually polystyrene). These insoluble carriers contain a group that will react with the carboxylic group to form a bond that is stable to the elongation conditions but readily cleaved later. Examples of which are: chloro- or bromomethyl resin, hydroxymethyl resin, trityl resin and 2-methoxy-4-alkoxy-benzylalcohol resin.

Many of these resins are commercially available with the desired C-terminal amino acid already incorporated. Alternatively, the amino acid can be incorporated on the solid support by known methods (Wang, S.-S., J. Am. Chem. Soc., (1973), 95, 1328; Atherton, E.; Shepard, R. C. “Solid-phase peptide synthesis; a practical approach” IRL Press: Oxford, (1989); 131-148, herein incorporated by reference). In addition to the foregoing, other methods of peptide synthesis are described in Stewart and Young, “Solid Phase Peptide Synthesis”, 2nd ed., Pierce Chemical Co., Rockford, Ill. (1984); Gross, Meienhofer, Udenfriend, Eds., “The Peptides: Analysis, Synthesis, Biology”, Vol. 1, 2, 3, 5, and 9, Academic Press, New-York, (1980-1987); Bodansky et al., “The Practice of Peptide Synthesis” Springer-Verlag, New-York (1984), herein incorporated by reference.

The P1′ fragments R⁴—S(O)ₘNH₂ are coupled to the P1, P2-P1 or P3-P2-P1 fragments in the presence of a coupling agent under standard conditions. Although several commonly used coupling agents can be employed, TBTU and HATU have been found to be practical. Alternatively, an azalactone of formula (II):

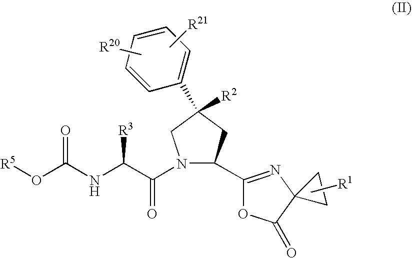

may be treated by the amide anion (IIIa):

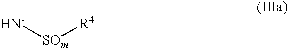

as described hereinabove, to effect the coupling reaction and prepare compounds of formula (I). The azalactone is readily prepared from the precursor carboxylic acid by treatment with a dehydrating agent such as isobutylchloroformate or the like, as shown in Scheme II below.

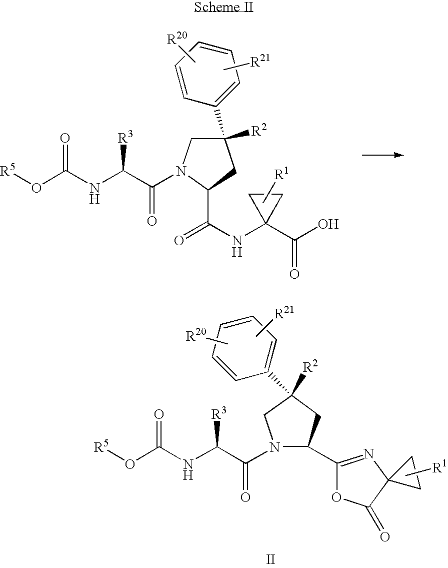

### Synthesis of P1 Fragments

P1 moieties of compounds of Formula (I) are prepared using the protocols outlined in WO 00/59929, published Oct. 12, 2000, and WO 00/09543, published on Feb. 24, 2000, herein incorporated by reference. In particular, reference is made to pages 33-35, Example 1 of WO00/59929 and Pages 56-69, Examples 9 to 20 of WO00/09543 for the preparation of 1-aminocyclopropanecarboxylic acid P1 moieties.

### Synthesis of P1′ Fragments

P1′ fragments of formula R⁴SO₂NH₂ are available commercially or are prepared by known methods or by procedures described in the following examples.

### Synthesis of P2 Fragments

Briefly, the proline intermediates can be readily made via oxidation of commercially available or easily prepared N-protected hydroxyproline esters. Oxidation of the hydroxyl group to give the corresponding 4-ketoproline analog can be performed using a variety of reagents including TPAP/NMO, Swern or other DMSO activation methods, or TEMPO based methods (for example, see: Tetrahedron 1978, 34, 1651-1660, J. Org. Chem., 2001, 66, 3593-3596 and J. Org. Chem. 2003, 68, 4999-5001, herein incorporated by reference.)

The protected 4-ketoproline esters can then be subsequently reacted with Grignard type reagents which are made in situ via magnesium-halogen exchange reactions. (For example see: P. Knochel et. al., Angew. Chem. Int. Ed., 2004, 43, 2-5 and Angew. Chem. Int. Ed., 2003, 42, 4302-4320, herein incorporated by reference.) These reagents add stereoselectively to produce 4-cis-hydroxy-4-phenyl-L-prolinate derivatives. (For example see: V. Hruby et. al., J. Org. Chem., 2001, 66, 3593-3596, herein incorporated by reference). The hydroxyl group can be converted to the corresponding ether by treatment with a base and an alkylating reagent (for example when R²═OMe, iodomethane can be used).

In the case of the 4-bromophenyl proline intermediate, 4-iodobromophenyl and 4-boronate esterphenyl, subsequent carbon-carbon bond formation can be effected by a variety of cross-coupling methodologies with various metal nucleophile partners in the case of the 4-iodo or 4-bromo derivatives or with aryl or heteroaryl halides (I, Br or Cl) in the case of the 4-boronate esterphenyl. Some of the typical methods for these coupling include: Suzuki reaction, Stille reaction, Hiyama reaction, and other metal-catalyzed cross-coupling reactions. (For reviews of metal-catalyzed cross-coupling reactions, see: Metal-catalyzed Cross-Coupling Reactions: Diederich, F., Stang, P., Eds.; Wiley-VCH: New York, 1998 and Cross-coupling Reactions: A Practical Guide; Miyaura, N., Ed., Topics in Current Chemistry Series 219; Springer-Verlag: New York, 2002 and Handbook of Organopalladium Chemistry for Organic Synthesis; Negishi, E,. Ed.; Wiley-Interscience: New York, 2002, herein incorporated by reference.). Furthermore, the coupling of the 4-iodobromophenyl and 4-bromophenyl proline can be accomplished via a decarboxylative coupling of a number of 2-carboxylic acid heterocycles (see: Forgione, Bilodeau et. al. J. Am. Chem. Soc. 2006, 128, 11350, herein incorporated by reference).

### Synthesis of P3 Fragments

The P3 carbamate fragments wherein R⁵ is B—O—C(═O)— are prepared as described in WO 03/064416, herein incorporated by reference.

### EXAMPLES

Other features of the present invention will become apparent from the following non-limiting examples which illustrate, by way of example, the principles of the invention. As is well known to a person skilled in the art, reactions are performed in an inert atmosphere (including but not limited to nitrogen or argon) where necessary to protect reaction components from air or moisture. Temperatures are given in degrees Celsius (° C.). Solution percentages and ratios express a volume to volume relationship, unless stated otherwise. Flash chromatography is carried out on silica gel (SiO₂) according to the procedure of W. C. Still et al., J. Org. Chem., (1978), 43, 2923. Mass spectral analyses are recorded using electrospray mass spectrometry. Analytical HPLC is carried out under standard conditions using a Combiscreen ODS-AQ C18 reverse phase column, YMC, 50×4.6 mm i.d., 5 μM, 120 Å at 220 nM, elution with a linear gradient as described in the following table (Solvent A is 0.06% TFA in H₂O; solvent B is 0.06% TFA in CH₃CN):

| Time (min) | Flow (mL/min) | Solvent A (%) | Solvent B (%) |
|---|---|---|---|
| 0 | 3.0 | 95 | 5 |
| 0.5 | 3.0 | 95 | 5 |
| 6.0 | 3.0 | 50 | 50 |
| 10.5 | 3.5 | 0 | 100 |

Abbreviations used in the examples include

AcOH: acetic acid;

Bn: benzyl;

Boc: tert-butyloxycarbonyl {Me₃C—O—C(O)};

brosyl: p-bromobenzenesulfonyl;

CDI: N,N′-Carbonyldiimidazole;

DBU: 1,8-diazabicyclo[5.4.0]undec-7-ene;

DCC: 1,3-dicyclohexylcarbodiimide;

DCM: dichloromethane;

DIPEA: diisopropylethylamine;

DMAP: 4-dimethylaminopyridine;

DMBA: 1,3-dimethylbarbituric acid;

DME: 1,2-dimethoxyethane;

DMF: dimethylformamide;

DMSO: dimethylsulfoxide;

EDTA: ethylenediaminetetraacetic acid;

Et: ethyl;

EtOH: ethanol;

EtOAc: ethyl acetate;

Et₂O: diethyl ether;

HATU: [O-7-azabenzotriazol-1-yl)-1,1,3,3-tetramethyluronium hexafluorophosphate];

HPLC: high performance liquid chromatography;

IBCF: iso-butyl chloroformate;

LAH: lithium aluminum hydride;

LiHMDS: lithium hexamethyldisilazide;

Me: methyl;

MeOH: methanol;

MS: mass spectrometry;

NaHMDS: sodium hexamethyldisilazide;

NMO: N-methylmorpholine-N-oxide;

NMP: N-methylpyrrolidone;

Pr: propyl;

t_{R}: retention time;

TBAF: tetra-n-butylammonium fluoride;

TBDMSCI: tert-butyldimethylsilyl chloride;

TBTU: 2-(1H-benzotriazole-1-yl)-1,1,3,3-tetramethyluronium tetrafluoroborate;

TEA: triethylamine;

TEMPO: 2,2,6,6-tetramethyl-1-piperidinyloxy, free radical

TFA: trifluoroacetic acid;

THF: tetrahydrofuran;

TPAP: tetra-n-propylammonium perruthenate;

Tris/HCl: tris(hydroxymethyl)aminomethane hydrochloride;

Ts: tosyl (p-methylbenzenesulfonyl);

RT: room temperature

### Example 1

### Synthesis of P3 Carbamate Fragment 1a

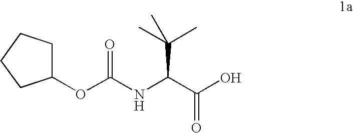

The P3 carbamate fragment 1a was prepared as described in WO 03/064416, herein incorporated by reference. It will be apparent to one skilled in the art that analogous P3 carbamate fragments in which the cyclopentyloxycarbonyl group has been replaced by another R⁵ substituent as defined herein and/or the tert-butyl group has been replaced by another R³ substituent as defined herein may be prepared using an analogous procedure. The preparation of analogous P3 urea fragments wherein R⁵ is B—NH—C(═O)— is described in WO 03/064456, herein incorporated by reference. Such fragments may be readily substituted for the P3 carbamate fragments in the examples below, to provide compounds of formula (I) wherein R⁵ is B—NH—C(═O)—.

### Example 2

### Synthesis of P1′ Fragments 2d and 2g

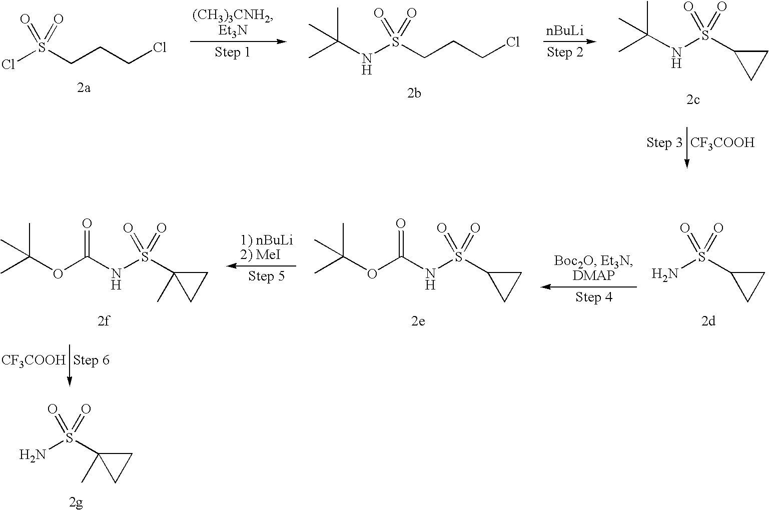

Cyclopropanesulfonamide can be prepared by amination of cyclopropanesulfonyl chloride, according to the literature reference of J. King et al., J. Org. Chem., 1993, 58, 1128-1135, herein incorporated by reference, or as set out below.

### Step 1:

A dry 3 L 3-neck flask equipped with a magnetic stir bar, addition funnel and argon inlet was flushed with argon, then charged with 3-chloropropanesulfonyl chloride 2a (100.48 g, 0.57 mol, 1.0 eq). Anhydrous dichloromethane (900 mL) was transferred into the flask via cannula, the mixture was cooled in an ice/water bath and tert-butylamine (72 mL, 0.68 mol, 1.2 eq) was added. The mixture was stirred 15 minutes then a solution of triethylamine (158 mL, 1.13 mol, 2.0 eq) in anhydrous dichloromethane (100 mL) was added dropwise over 45 minutes and stirring was continued for 1 h. The mixture was diluted with dichloromethane (500 mL) and washed with 1N HCl (3×400 mL) and brine. The organic layer was dried over sodium sulfate, filtered and evaporated to dryness to give compound 2b as an orange-beige solid (107.04 g, 88% yield).

### Step 2:

A dry 5 L 3-neck flask equipped with a magnetic stir bar, argon inlet and 2 addition funnels was flushed with argon and anhydrous THF (1.5 L) was transferred into the flask via cannula and cooled to −78° C. Compound 2b (96.73 g, 0.453 mol, 1.0 eq) was dissolved in anhydrous THF (390 mL) and the solution was transferred into one of the addition funnels. n-Butyllithium solution (2.5 M in hexanes, 390 mL, 0.975 mol, 2.15 eq) was transferred to the other addition funnel and the solutions in the addition funnels were added to the flask simultaneously over 4 hours. When addition was complete, the mixture was allowed to warm to room temperature. Once the internal temperature reached ˜0° C., the reaction was quenched by dropwise addition of saturated NH₄Cl solution (200 mL). The THF was removed under vacuum and the residue was diluted with CH₂Cl₂ (2 L) and water (1 L). The layers were separated and the organic layer was washed with water (2×1 L) and brine (800 mL), dried over sodium sulfate, filtered and evaporated to dryness. Compound 2c was obtained as an orange-beige solid (77.32 g, 96% yield).

### Step 3:

A 2 L flask equipped with a magnetic stir bar and condenser was charged with compound 2c (82.53 g, 0.466 mol, 1.0 eq), dichloromethane (400 mL) and trifluoroacetic acid (460 mL, 5.97 mol, 13 eq). The mixture was heated to reflux for 2 h, allowed to cool, and evaporated and co-evaporated several times with CH₂Cl₂ to remove most of the TFA. The crude product was dissolved in 95:5 CH₂Cl₂:MeOH and NH₄OH and was purified by silica gel column:chromatography (94:5:1 CH₂Cl₂:MeOH:NH₄OH). Compound 2d was obtained as a beige solid (46.38 g, 78% yield).

### Step 4:

To the solid cyclopropanesulfonamide 2d (1.51 g; 12.46 mmol) was added in sequence: di-t-butyl-dicarbonate (3.26 g; 14.95 mmol) dissolved in anhydrous dichloromethane (15 mL), triethylamine (2.6 mL; 18.65 mmol) and dimethylaminopyridine (76 mg; 0.622 mmol). The resulting solution was stirred at room temperature overnight and subsequently evaporated to near dryness. The residue was diluted with EtOAc, washed with 1N aq. HCl (3×) and brine (1×), dried (MgSO₄), filtered and evaporated to dryness to provide the Boc-cyclopropylsulfonamide product 2e as a white solid (2.6 g; 94% yield).

### Step 5:

To a cooled solution (−78° C.) of the Boc-cyclopropanesulfonamide 2e (500 mg; 2.26 mmol) in anhydrous THF (15 mL) was added dropwise n-BuLi (2.1 mL; 5.20 mmol) and the mixture was allowed to stir 1 h at −78° C. Two portions of methyl iodide (each 280 μL; 4.52 mmol) were added with a one hour interval and the reaction mixture was allowed to warm slowly to RT and stir at RT overnight. The reaction mixture was adjusted to pH 3 with 1N aq. HCl and the product was extracted with EtOAc (3×). The combined EtOAc extracts were washed with brine (1×), dried (MgSO₄), filtered and evaporated to dryness to provide the crude alkylated product 2f as a light yellow oil. The crude material was purified by flash chromatography over silica gel with hexane:EtOAc (9:1) as eluent to provide pure product 2f as a yellow oil (151.8 mg; 29% yield).

### Step 6:

To a solution of the Boc-1-methylcyclopropanesulfonamide 2f (151.8 mg: 0.65 mmol) in dichloromethane (6 mL) was added trifluoroacetic acid (6 mL) and the mixture allowed to stir at RT for 3.5 h. Evaporation to dryness under high vacuum provided the deprotected material 2g as an off-white wax like solid (79.1 mg, 91% yield).

### Example 3

### Synthesis of P1-P1′ Fragments 3c and 3d

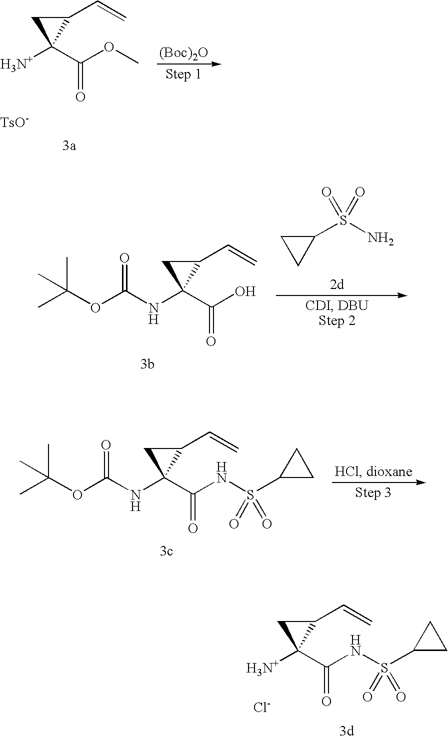

### Step 1:

To a solution of compound 3a (prepared using an analogous procedure to the methodology disclosed in WO 00/09543, herein incorporated by reference) (12 g, 38.29 mmol) in a mixture of THF (50 mL) and 1 N aq. NaOH (85 mL, 85.00 mmol) was added Boc anhydride (10 g, 45.95 mmol). The reaction mixture was stirred at RT for 4 days. The pH was periodically adjusted to 9 by adding more NaOH. The THF was then removed in vacuo and the aqueous layer was washed with ether (3×150 mL) and then cooled to 0° C. for the slow addition of 1 N aq. HCl until pH 3-4 was obtained. The aqueous layer was then extracted with EtOAc (3×150 mL) and the combined organic extracts were successively washed with water (3×100 mL) and brine. After drying over MgSO₄, filtration and concentration, 5.16 g of the desired Boc-protected intermediate 3b was isolated.

### Step 2:

To a solution of acid 3b (567 mg, 2.49 mmol), in THF (20 mL), was added CDI (515 mg, 3.17 mmol). The resulting solution was stirred for 30 min, refluxed for 30 min and allowed to cool down to RT. Cyclopropylsulfonamide 2d (455 mg, 3.76 mmol) was added followed by the addition of DBU (0.75 mL, 5.02 mmol) and the reaction was stirred 12 h. The THF was removed in vacuo and the residue was diluted with EtOAc, washed with 1 M HCl (2×100 mL) and brine, dried (MgSO₄) and purified by flash chromatography (elution conditions: 70:30 hexane/EtOAc) to afford 682 mg (82% yield) of compound 3c as a white solid.

### Step 3:

Compound 3c (375 mg, 1.13 mmol), in 8 mL of 4M HCl/dioxane, was stirred at room temperature. After 30 minutes a solid appeared. MeOH was added until the solid dissolved completely and the reaction mixture was stirred for an additional 30 min. Before evaporation of the solvent, the residue was dried under vacuum to afford the amine salt 3d as an off white solid.

### Example 4A

### Synthesis of P2 Intermediate 4e

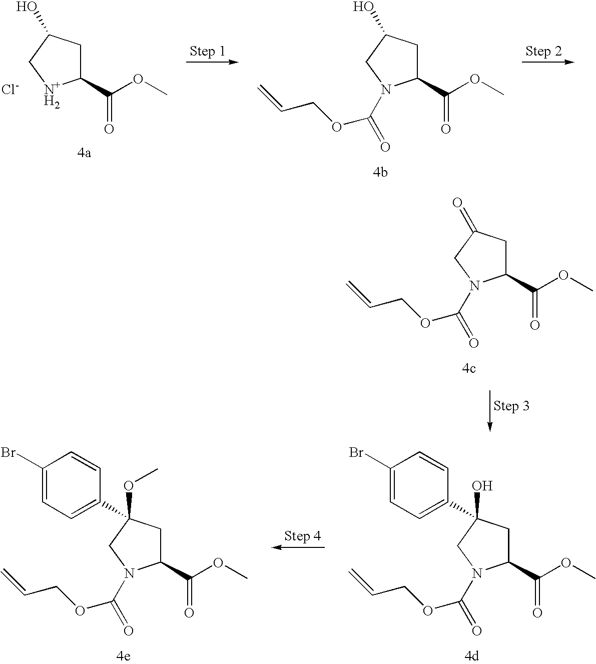

### Step 1:

To a solution of commercially available trans-4-hydroxyproline methyl ester HCl salt 4a (15.1 g, 83.14 mmol, 1 eq) in DCM (200 mL) at 0° C. was added triethylamine (26.7 mL, 191.2 mmol, 2.3 eq) followed by allyl chloroformate (9.7 mL, 91.45 mmol, 1.1 eq). The resulting mixture was stirred at 0° C. for 30 minutes and allowed to warm to RT over 2 hrs. A saturated solution of NaHCO₃ (100 mL) was added and the mixture allowed to stir at RT for 5 minutes. The phases were separated and the aqueous layer was then extracted with DCM (2×). The combined organic phases were then washed with 1N HCl (aq) and brine and dried over MgSO₄. The dried organic phase was then filtered and concentrated in vacuo to afford compound 4b (18 g, 94% yield) as a pale yellow oil that was used as is for the next step.

### Step 2:

To distilled oxalyl chloride (11.3 mL, 130 mmol) in dichloromethane (850 mL) at −70° C. was added dropwise a solution of anhydrous DMSO (20 mL, 283 mmol) in dichloromethane (50 mL). After 15 minutes at −70° C., a solution of compound 4b (27.05 g, 118 mmol) in dichloromethane (100 mL) was added. The mixture was stirred at −70° C. for 30 minutes. Next, triethylamine (82.2 mL, 590 mmol) was added and the resultant solution stirred at −70° C. for 15 minutes and then at RT until the solution became clear (5 h). The reaction was diluted with water and separated. The aqueous phase was re-extracted with dichloromethane. The combined organic phases were washed with sat. NaHCO₃ (aq) (2×), followed by water and sat. brine. The organic phase was dried over MgSO₄, filtered and concentrated to give an oil.

This material was purified by flash chromatography (SiO₂, solvent: 5-10% EtOAc/hexane) to afford compound 4c as a yellow oil (16.6 g, 62% yield). MS: ES⁻: 226.0.

### Step 3:

To a solution of 1-bromo-4-iodobenzene (8.72 g, 30.81 mmol) in anhydrous THF (70 mL) at 0° C. under a nitrogen atmosphere was added i-PrMgCl—LiCl [prepared as in: P. Knochel, Angew. Chem. Int. Ed., 2004, 43, 2-5, herein incorporated by reference.] (0.82M in THF, 37.6 mL, 30.81 mmol, 1 equiv). The solution turned milky after 2 minutes and reaction was continued at 0° C. for 30 minutes until completion of the magnesium-iodide exchange reaction. To the magnesium-iodide exchange reaction mixture was added the ketone 4c (7.0 g, 30.81 mmol) in 100 mL of anhydrous THF via cannulation (ca. 2 minutes). The resulting mixture was stirred at RT for 2 h. The reaction mixture was quenched with sat. NH₄Cl (300 mL) and then diluted with dichloromethane (3×). The organic phases were dried (MgSO₄), filtered and concentrated to afford an orange oil. This material was purified by column chromatography (SiO₂, eluting with a gradient of 20% to 30% EtOAc/hexanes) to give alcohol 4d as a pale orange oil (6.69 g, 57% yield). MS: (M+Na)+; 406 and 408 (Br isotope).

### Step 4:

The alcohol 4d (6.69 g, 17.41 mmol) was dissolved in anhydrous DMF (120 mL) and cooled to 0° C. before iodomethane (21.7 mL, 348 mmol, 20 eq) was added. This was followed with the addition of solid KH (previously washed with hexanes and dried under vacuum; 1.40 g, 34.8 mmol). A saturated aqueous solution of NH₄Cl (100 mL) was added, followed by the addition of water. The mixture was extracted with a mixture of Et₂O/hexanes (1:1), dried over MgSO₄, filtered and concentrated to afford an orange oil. This material was purified by column chromatography (SiO₂, eluent: 40% EtOAc/hexanes, R_{f}=0.4) to give the desired methyl ether 4e (6.9 g, 88% yield). MS: (M+H)⁺; 420 and 422 (Br isotope).

### Example 4B

### Synthesis of P2 Intermediate 4g

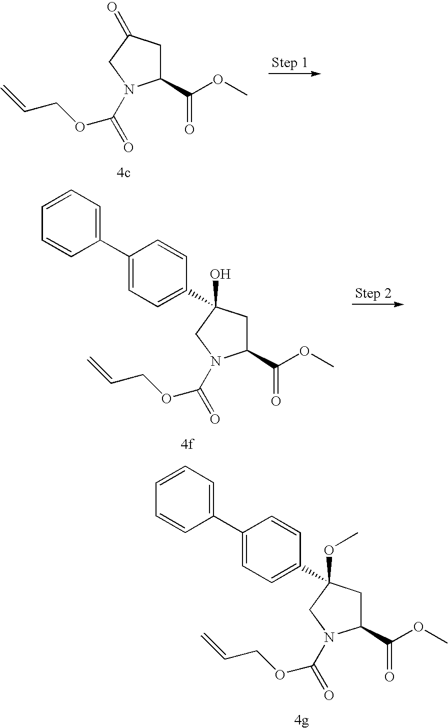

### Step 1:

To a solution of 4-iodobiphenyl (2.47 g, 8.8 mmol) in anhydrous THF (80 mL) at 0° C. was added iPrMgCl—LiCl (10.36 mL, 8.8 mmol, 0.85M in THF). After 1 h, the ketone 4c (2.0 g, 8.8 mmol) was added in anhydrous THF (60 mL) and stirred for 1 h at RT. To this mixture was added a saturated solution of NH₄Cl (60 mL) before extraction with dichloromethane (3×). The organic phases were dried over MgSO₄, filtered and concentrated. This material was purified by flash chromatography (eluting with 15-30% EtOAc/hexanes) to give the desired alcohol 4f (1.67 g, 50% yield) as a white solid.

### Step 2:

The alcohol 4f (1.67 g, 4.38 mmol) was dissolved in anhydrous DMF (50 mL) cooled to 0° C. This solution was treated with iodomethane (5.45 mL, 88 mmol) before the addition of KH (263 mg, 6.6 mmol, previously washed with hexanes) was added. The reaction was stirred at 0° C. for 1.5 h before being quenched carefully with a saturated solution of NH₄Cl (100 mL) and water. The mixture was extracted with ether/hexanes (1:1), dried over MgSO₄, filtered and concentrated. The crude material was purified over silica gel eluting with a gradient of 15-20% EtOAc/hexanes to give the methyl ether 4g (1.54 g, 89% yield) as a colorless oil.

### Example 5

### Synthesis of Dipeptide Intermediate 5c

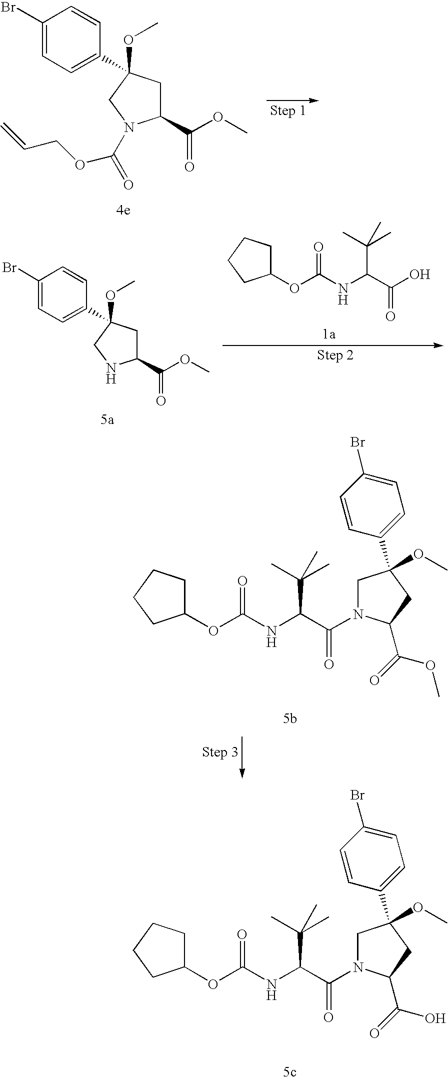

### Step 1:

To a solution of ether 4e (Example 4A) (3.34 g, 8.39 mmol) in anhydrous THF (50 mL) was added 1,3-dimethylbarbituric acid (2.62 g, 16.8 mmol, 2 equiv.) and Pd(PPh₃)₄ (291 mg, 0.25 mmol). The reaction mixture was stirred at RT for 16 h, then was diluted with EtOAc (100 mL) and washed with 1N HCl (aq) (3×). The combined aqueous phases were combined and basified using 4N NaOH to a final pH of 13, then extracted with dichloromethane (3×). The combined organic phase from this extraction was then dried (MgSO₄), filtered and concentrated to give the free amine 5a (2.37 g, 90% yield) as a pale orange oil. This material was used as such in the next step.

### Step 2:

To a solution of amine 5a (2.37 g, 7.54 mmol) in anhydrous DMF (40 mL) was added sequentially DIPEA (6.57 mL, 38 mmol, 5 eq), compound 1a (Example 1) (2.39 g, 9.81 mmol, 1.3 eq) and HATU (3.73 g, 9.81 mmol, 1.3 eq). The resulting solution was stirred at RT and monitored by HPLC until completion. To the reaction mixture was added EtOAc (300 mL) and water (100 mL). The separated organic phase was washed with saturated NaHCO₃ (2×), water (1×) and finally sat. brine (1×). The organic phase was dried over MgSO₄, filtered and concentrated to afford an orange oil that was further purified by column chromatography (SiO₂, eluent: 40% EtOAc/hexanes, Rf=0.42) to give dipeptide 5b as a white solid (3.52 g, 87% yield). MS: (M+H)⁺; 539 and 541 (Br isotope).

### Step 3:

To a solution of dipeptide 5b (1.0 g, 1.85 mmol) in THF/MeOH (15 mL, 2:1 mixture) at RT was added 1N NaOH (2.8 mL, 2.8 mmol). The solution was stirred several hours until reaction was complete, then was acidified to pH ˜2 with 1N HCl and the aqueous phase extracted with dichloromethane (3×). The combined organic layers were dried over MgSO₄, filtered and concentrated to afford acid 5c (0.98 g, 100% yield) as a white crystalline solid. MS: (M+H)⁺; 525 and 527 (Br isotope).

### Example 6

### Synthesis of Dipeptide Intermediate 6g

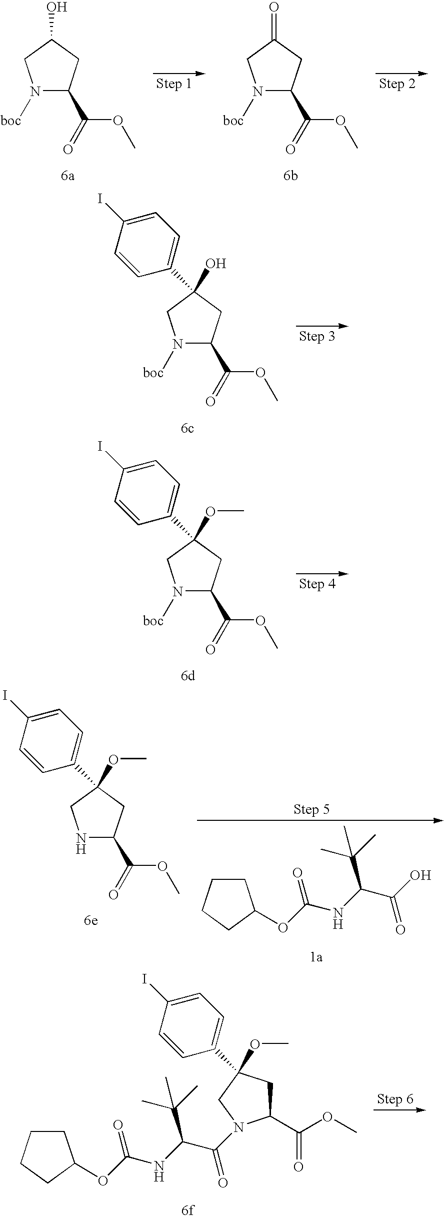

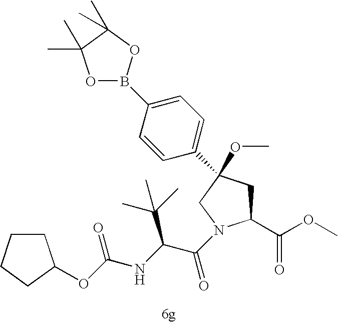

### Step 1:

To a solution of the alcohol 6a (3.0 g, 12.23 mmol) in anh. DCM (50 ml) at 0° C. was added the trichloroisocyanuric acid (2.99 g, 12.84 mmol). The heterogenous mixture was stirred for 1 minute (partial dissolution of trichloroisocyanuric acid) then TEMPO (58 mg, 0.37 mmol) was added. The reaction mixture was then allowed to warm-up to RT and monitored by TLC. After the reaction was complete (15 minutes), EtOAc (100 ml) was added, the organic phase was washed with a saturated solution of NaHCO₃ (2×), 1N HCl (2×), 10% Na₂S₂O₃ solution (3×), once with brine and it was then dried over MgSO₄ and filtered. Solvent evaporation afforded the desired ketone 6b (2.92 g, 98% yield) as a pale orange oil.

### Step 2:

To a solution of phenyl-diiodide (12.53 g, 37.98 mmol) in THF at 0° C. was added i-PrMgCl—LiCl (46.3 mL, 0.82 M, 37.98 mmol). This mixture was stirred at 0° C., HPLC monitoring showed that the Mg/l exchange was complete after 15 minutes, and ketone 6b (7.7 g, 31.65 mmol) in THF (70 mL) was then added, and the resulting mixture was stirred for 3 hrs (completion observed by TLC) at 0° C. A saturated solution of NH₄Cl was added, and was extracted with DCM (3×). The combined organic phases were dried over MgSO₄, filtered and solvent evaporation afforded an orange oil that was purified by flash column chromatography to give the desired alcohol 6c as a thick yellow oil (16.5 g, 72% yield).

### Step 3:

Alcohol 6c (10.2 g, 22.81 mmol) was dissolved in anhydrous DMF (240 mL), cooled to 0° C. Iodomethane (28.4 mL, 456 mmol) was then added followed by KH (1.83 g, 45.6 mmol, pre-washed with hexanes) in one portion. HPLC monitoring showed that the reaction was complete after 30 minutes. A saturated solution of NH₄Cl was added, followed by water, and it was extracted with a 1:1 mixture Et₂O/hexanes, dried over MgSO₄, and filtered. Solvent evaporation afforded the desired product 6d which was purified by flash column chromatography (9.0 g, 87% yield).

### Step 4:

A 4M HCl/dioxane solution was added to 6d (8.95 g, 19.4 mmol) and the reaction followed by RP-HPLC. It was then concentrated reaction under high vacuum with no heat and employed crude 6e in subsequent reaction without further purification.

### Step 5:

To a solution of the free amine 6e (3.3 g, 9.1 mmol) in DMF (50 mL) was added DIPEA (8.0 mL, 46 mmol), the carboxylic acid 1a (2.9, 11.9 mmol) and HATU (4.5 g, 11.9 mmol). The resulting mixture was stirred at RT for 1 hr (completion checked by HPLC). EtOAc (250 ml) then water (100 ml) were added. After phase separation the organic layer was washed with sat'd NaHCO₃ (2×), water (1×) and brine. It was then dried over MgSO₄, and filtered. Solvent evaporation afforded the crude product 6f. The mixture was thus dissolved in THF (50 ml), 15 ml of 1N HCl was added followed by MeOH (˜3 ml, for complete dissolution). It was then stirred overnight. EtOAc (250 ml) then water (100 ml) were added. After phase separation the organic layer was washed with sat'd NaHCO₃ (2×), water (1×) and brine. It was then dried over MgSO₄, filtered and solvent evaporation followed by flash column chromatography purification (eluent: 40% EtOAc/hexanes) to the desired product 6f (5.30 g, 99% yield) as a pale beige crystalline solid.

### Step 6:

In a round-bottom flask was added iodide 6f (4.0 g, 6.8 mmol), bispinocolatoborane (2.3 g, 8.9 mmol) and potassium acetate (1.9 g, 20.5 mmol). DMSO (42 ml) was added and the solution was bubbled with Ar for 30 minutes and then the PdCl₂dppf (557 mg, 0.68 mmol) was added. It was bubbled with Ar for another 5 minutes and then stirred at 80° C. for 14 h, under Argon. HPLC monitoring showed the reaction to be complete and clean. TLC: Rf=0.51 in 50% EtOAc/hexanes. Water (50 ml) was added followed by a 1:1 mixture of Et₂O/hexanes (300 ml). After phase separation the aqueous layer was washed with a 1:1 mixture of Et₂O/hexanes (2×100 ml). The combined organic phases were dried over MgSO₄, filtered, and silica was added. After solvent evaporation it was purified by flash column chromatography (CombiFlash) to afford the desired product 6g (2.61 g, 65% yield) as a white crystalline solid. MS ES+=587.3, ES−=585.3.

### Example 7

### Synthesis of Compound 1001

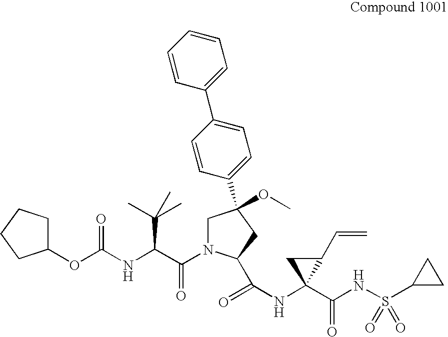

The compound was prepared using three alternative routes as described in Examples 7A, 7B and 7C below. As will be appreciated by one skilled in the art, these procedures are also applicable for the preparation of other compounds of formula (I).

### Example 7A

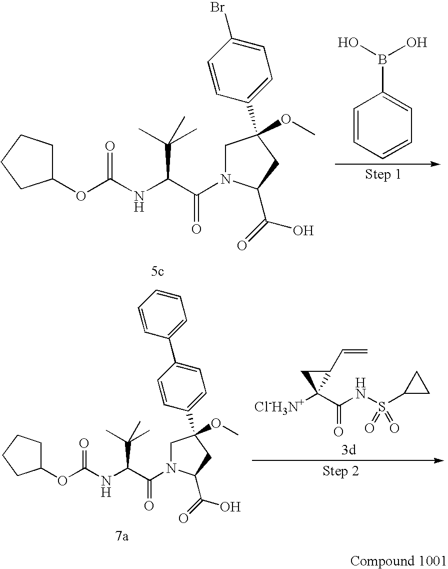

### Step 1:

To acid 5c (0.62 g, 1.18 mmol) in DME (12 mL) was added successively phenylboronic acid (0.2 g, 1.65 mmol) and aqueous sodium carbonate (2M, 6 mL). Nitrogen gas was bubbled through the resulting solution for 10 min before Pd(PPh₃)₄ (26.2 mg, 0.02 mmol) was added. Nitrogen gas was bubbled through the mixture for an additional 5 minutes then the mixture was refluxed for 5 h. The mixture was diluted with EtOAc (150 mL) and washed with water before being dried over MgSO₄, filtered and concentrated. The crude material was purified by flash chromatography (5% MeOH/dichloromethane) to afford compound 7a (291 mg, 47% yield) as an off-white solid.

### Step 2:

To the biphenyl acid 7a (291 mg, 0.56 mmol) in anhydrous DMF (10 mL) was added HATU (275 mg, 0.72 mmol), the amine hydrochloride 3d (Example 3) (0.72 mmol) and finally diisopropylethyl amine (485 μL, 2.8 mmol). The reaction was stirred for 5 h then diluted with EtOAc (150 mL) and washed with water (1×), 1N HCl (2×), and finally saturated brine. The organic phase was dried over MgSO₄, filtered and concentrated to give an oil. This material was purified by flash chromatography (60% EtOAc/hexanes) to afford the desired compound 1001 (240 mg, 59% yield) as a white solid. MS: (M+H)+; 735.4.

### Example 7B

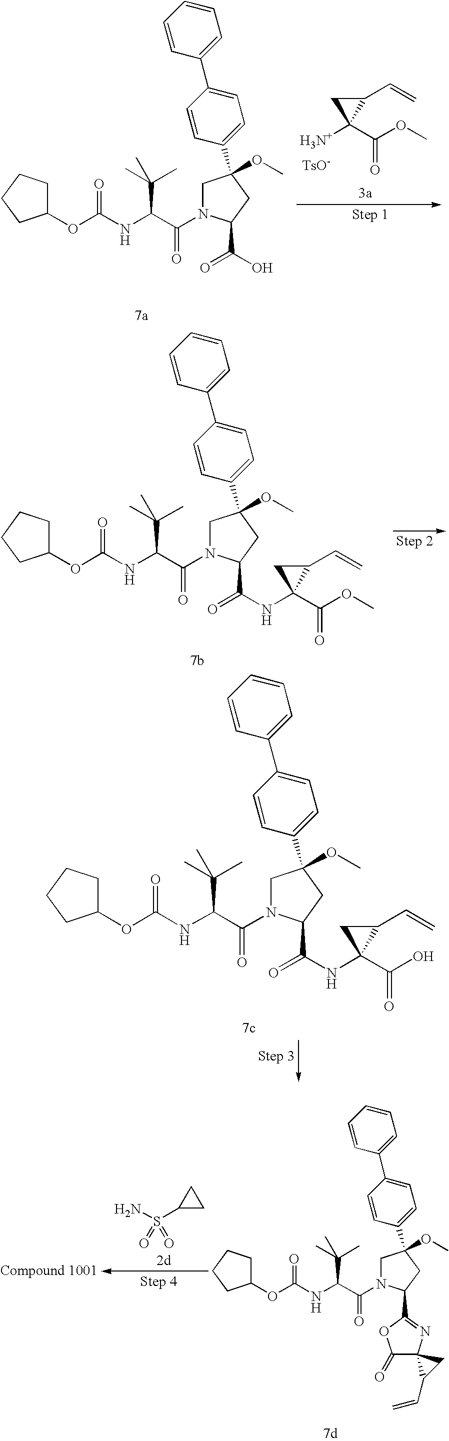

### Step 1:

A mixture of compound 7a (Example 7A) (assume 0.375 mmol) and HATU (171 mg; 0.45 mmol) was stirred for 30 minutes. To this mixture was added a solution of compound 3a (Example 3) (141.0 mg; 0.45 mmol) in acetonitrile (1 mL) and DIPEA (261 μL). The reaction mixture was stirred overnight then evaporated to dryness and diluted with EtOAc, washed with 10% citric acid (2×), water (2×), saturated NaHCO₃ (2×), water (2×) and brine (1×). The organic phase was dried (MgSO₄), filtered and evaporated to dryness to provide the product 7b as an off-white foam (222 mg; 92% yield).

### Step 2:

To the starting ester 7b (222.2 mg; 0.34 mmol) dissolved in a mixture of THF (1 mL), MeOH (0.5 mL) and water (0.5 mL) was added 1N NaOH (1.3 mL) and the mixture allowed to stir at RT overnight. The mixture was evaporated to near dryness and the resulting paste dissolved in a mixture of EtOAc and 1N HCl (pH of the aqueous layer was ˜3). The product was extracted into EtOAc (3×), and the combined extracts were washed with water (2×) and brine (1×), dried (MgSO₄), filtered and evaporated to dryness to provide the product 7c as an off-white foam (211 mg; 97% yield).

### Step 3:

To an ice cooled solution of the acid component 7c (211 mg, 0.34 mmol) in dichloromethane (3 mL) containing triethylamine (154 μL; 1.10 mmol) was added dropwise isobutylchloroformate (65 μL; 0.50 mmol). The reaction was stirred at 0° C. for 1 hour and at RT for 2 hrs. The mixture was loaded onto a silica flash column and purified by elution with hexane:EtOAc (9:1 then 8:2) to provide the azalactone product 7d as a white foam like solid (154.7 mg; 76% yield).

### Step 4:

To a cooled solution (−15 to −20° C.) of the sulfonamide 2d (45.8 g; 0.378 mmol) in anhydrous THF (1 mL) was added, in one portion, a solution of LiHMDS (1.0 M in THF; 302 μL). The yellow solution was stirred at the bath temperature for 5 minutes, then at RT for 20 minutes and subsequently cooled to −10 to −15° C. A solution of the azalactone 7d (154.7 mg; 0.252 mmol) in anhydrous THF (2 mL) was added dropwise and the reaction mixture was allowed to slowly warm to RT and stir at RT overnight. The mixture was diluted with 1N HCl (pH˜3) and EtOAc and extracted 3× with EtAOc. The combined extracts were washed with water (2×) and brine (1×), dried (MgSO₄), filtered and evaporated to dryness to provide a white foam. The crude material was purified by flash chromatography (Eluent:Hexane:EtOAc; 6: 4) to provide the product, compound 1001, as a white foam (162 mg; 87% yield). Final purification of 15 mg was achieved by preparatory HPLC (Reverse phase: YMC, Combiscreen ODS-AQ, 50×20 mm ID S-5 micron, 120 A; λ=220 nm) using a linear gradient and 0.06% TFA CH₃CN/H₂O from 2-100% CH₃CN. The fractions were analyzed by analytical HPLC (Reverse phase: YMC, Combiscreen ODS-AQ, 50×4.6 mm ID S-5 micron, 120 A; λ=220 nm), pure fractions were combined, concentrated and lyophilized to provide compound 1001 as a white amorphous solid (11.1 mg).

### Example 7C

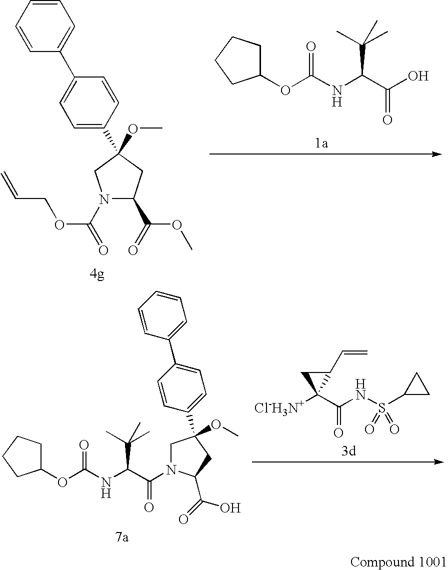

Compound 4g was deprotected, coupled to compound 1a and saponified using procedures analogous to those described in Example 5, steps 1, 2 and 3, to give compound 7a. Compound 7a was then converted to compound 1001 as described in Example 7A, step 2.

### Example 8

### Synthesis of Compound 1031

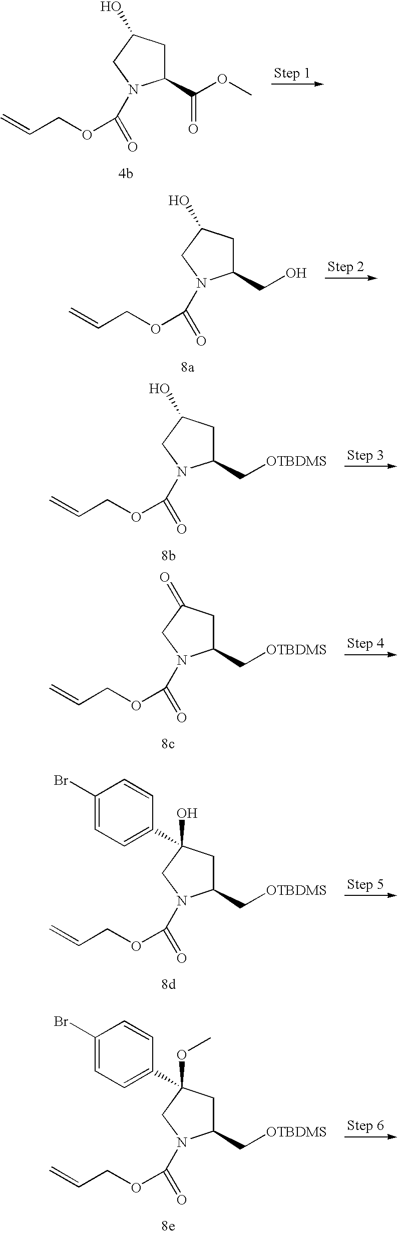

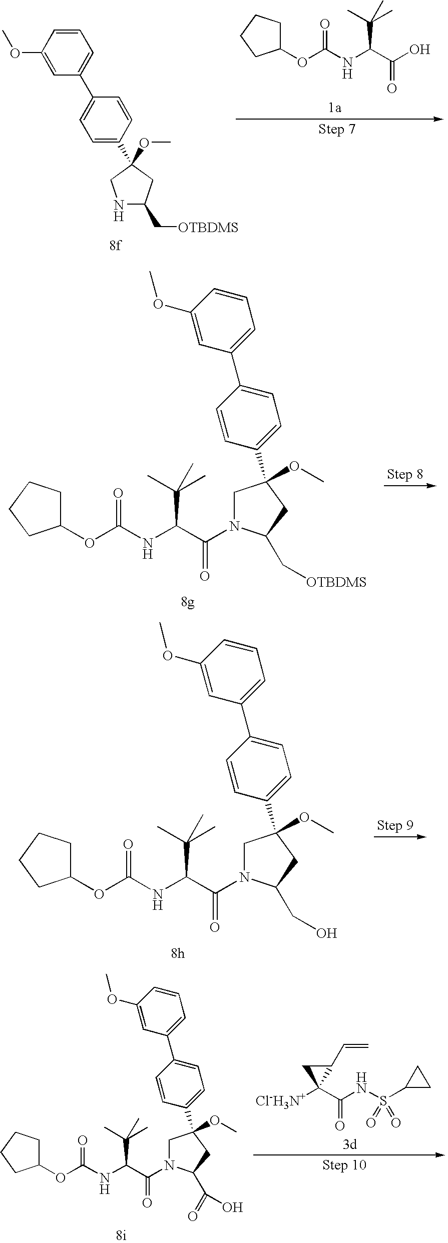

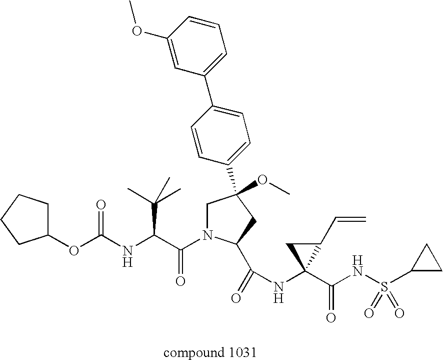

### Step 1:

To a solution of hydroxyproline 4b (12.62 g, 55.05 mmol) in anhydrous THF (100 mL) at 0° C. was added LiBH₄ (1.55 g, 71.6 mmol). The resulting mixture was stirred at 0° C. for 20 minutes and then allowed to warm to RT. To this mixture was slowly added water (50 mL) followed by dropwise addition of 4N HCl to reach pH=3. This solution was extracted with dichloromethane (3×), dried over MgSO₄, filtered and concentrated to dryness to afford diol 8a (7.53 g, 68% yield) as a colorless oil.

### Step 2:

To a solution of diol 8a (6.81 g, 33.8 mmol) in dichloromethane (60 mL) at 0° C. was added imidazole (2.53 g, 37.2 mmol), followed by tert-butyldimethyl-silyl chloride (5.6 g, 37.2 mmol). The resulting mixture was stirred at 0° C. for 10 minutes and then allowed to stir at RT (16 h). The reaction was quenched with sat. NaHCO₃ and the phases separated. The aqueous phase was extracted with dichloromethane (2×) and then dried over MgSO₄, filtered and concentrated to give an oil. The product was purified by flash chromatography (50% EtOAc/hexanes) to give the silyl ether 8b (6.76 g, 63% yield) as a clear oil.

### Step 3:

To a solution of silyl ether 8b (3.9 g, 12.36 mmol) in anhydrous dichloromethane (100 mL) at RT was added successively 4 A molecular sieves (oven dried, 3.9 g), NMO (2.17 g, 18.5 mmol), and TPAP (195 mg, 5% wt). The resulting mixture was stirred at RT for 3 h. The reaction mixture was concentrated under reduced pressure at RT and then filtered through a pad of silica gel, washing with 30% EtOAc/hexanes to give after concentration the desired ketone 8c (2.7 g, 70% yield) as a pale yellow oil.

### Step 4:

To a solution of 1-bromo-4-iodobenzene (2.44 g, 8.6 mmol) in anhydrous THF at 0° C., was added i-PrMgCl—LiCl complex (8.6 mL, 1.0M in THF). After 30 minutes, a solution of ketone 8c (1.5 g, 4.8 mmol) in anhydrous THF (30 mL) was added. The reaction mixture was stirred at RT (16 h) before being quenched with saturated NH₄Cl (100 mL) and extracted with dichloromethane (3×). The organic phases were dried over MgSO₄, filtered and concentrated. The crude material was purified by column chromatography (eluting with 15% EtOAc/hexanes) to afford alcohol 8d (1.1 g, 49% yield) as an oil. MS: (M+Na)⁺; 492.

### Step 5:

Alcohol 8d (1.1 g, 2.34 mmol) was dissolved in anhydrous THF (40 mL) at 0° C. and treated with iodomethane (0.73 mL, 11.7 mmol). To this solution was added KH (washed with hexanes) (281 mg, 7.0 mmol). The reaction was stirred at 0° C. for 1 h before being carefully quenched with water (15 mL). The mixture was extracted with dichloromethane (3×) and then dried over MgSO₄, filtered and concentrated to afford methyl ether 8e (1.13 g, 100% yield).

### Step 6:

Methyl ether 8e (150 mg, 0.31 mmol) was dissolved in DME (8 mL) and successively treated with 3-methoxyphenylboronic acid (66 mg, 0.43 mmol), aqueous Na₂CO₃ (3 mL, 2M in water), and 1,3-dimethylbarbituric acid (DMBA, 145 mg, 0.93 mmol). The resulting mixture was bubbled with N₂ for 30 minutes before Pd(PPh₃)₄ (21 mg, 0.02 mmol) was added. Nitrogen gas was bubbled through the reaction mixture for an additional 10 minutes, then the reaction was stirred at reflux for 16 h. Concentration of the mixture in vacuo gave a residue which was taken up into EtOAc and then washed with 1N NaOH (3×) and sat. brine. The organic phase was dried (MgSO₄), filtered and concentrated to give the free amine 8f (132 mg, 100% yield).

### Step 7:

To amine 8f (132 mg, 0.31 mmol) in DMF (4 mL) was added DIPEA (269 μL, 1.54 mmol). This solution was added to a pre-mixed solution of acid 1a (98 mg, 0.40 mmol) in DMF (2 mL) and HATU (153 mg, 0.40 mmol). The reaction mixture was stirred at RT for 2 h, then diluted with EtOAc and water. The phases were separated and the organic phase washed with sat. NaHCO₃ (2×), water (1×), and sat. brine (1×). The organic layer was dried over MgSO₄, filtered and concentrated to give the dipeptide 8g (167 mg, 100% yield) as an oil. This material was used directly in the following step.

### Step 8:

Dipeptide 8g (167 mg, 0.31 mmol) was dissolved in anhydrous THF (5 mL) at RT and treated dropwise with TBAF (0.62 mL, 0.62 mmol, 1.0 M in THF). The reaction was stirred 2 h until completion and then concentrated in vacuo. The crude material was purified by flash chromatography (eluting with 60% EtOAc/hexanes) to give alcohol 8h (74 mg, 45% yield).

### Step 9:

To alcohol 8h (74 mg, 0.14 mmol) in a mixture of CCl₄/CH₃CN/H₂O (1:1:1.5) at 0° C. was added sodium periodate (117.5 mg, 0.55 mmol) followed by ruthenium chloride hydrate (2.2 mg, 0.01 mmol). The reaction was stirred for 4 h, then ether was added and stirring was continued 10 minutes to precipitate RuO₂. The mixture was dried over MgSO₄, filtered, washed with ether, and concentrated to afford acid 8i (76 mg). This material was used as is in the following coupling step.

### Step 10:

Acid 8i (50 mg, 0.09 mmol) was dissolved in anhydrous DMF (2.5 mL) and treated with HATU (45 mg, 0.12 mmol) and DIPEA (79 μL, 0.45 mmol). To this solution was added neutralized salt 3d (0.12 mmol) in DMF (1 mL). The reaction was stirred at RT for 2 h before being directly purified by preparative HPLC to give the desired compound 1031 (9.75 mg, 14% yield). MS: (M+Na)+; 787.4 and (M−H)−; 763.4.

### Example 9

### Synthesis of Compound 1050

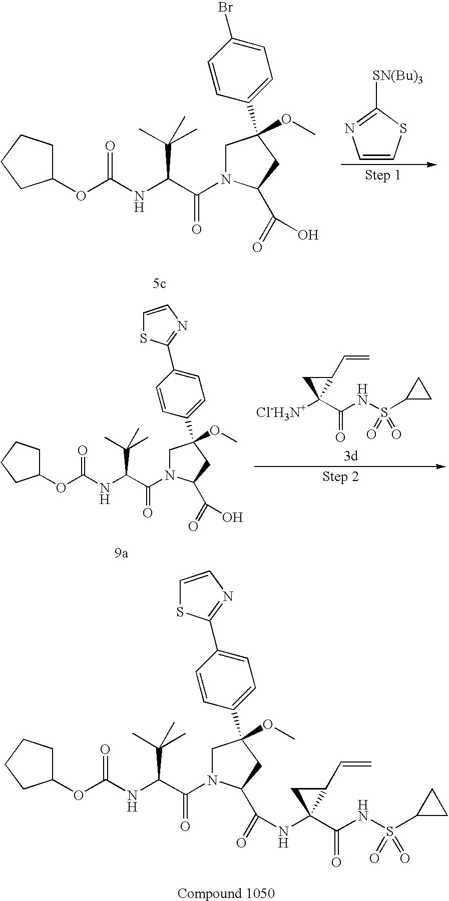

### Step 1:

To acid 5c (Example 5) (410 mg, 0.78 mmol) was added 2-tributylstannylthiazole (470 mg, 1.26 mmol) in anhydrous toluene (15 mL). This solution was bubbled with N₂ for 10 min and Pd(PPh₃)₄ (180 mg, 0.16 mmol) was added. The reaction mixture was bubbled with N₂ for an additional 10 minutes, then heated to reflux for 3.5 h, cooled and diluted with EtOAc (60 mL), and the organic layer washed with 1N NaOH (3×). The aqueous phase was acidified to pH˜4 with 4 N HCl and extracted with dichloromethane (3×). The combined phases were dried over MgSO₄, filtered and concentrated to afford compound 9a as an oil (397 mg, 96% yield). This material was dried under high vacuum and used as is in the next step.

### Step 2:

Acid 9a (397 mg, 0.75 mmol) was dissolved in anhydrous DMF (10 mL) and treated with HATU (370 mg, 0.97 mmol) and DIPEA (653 μL, 3.75 mmol). To this solution was added the amine hydrochloride salt 3d (Example 3) (0.97 mmol) previously neutralized with DIPEA (652 μL, 3.75 mmol) in DMF (2 mL). The reaction mixture was stirred at RT for 1 h. The crude reaction mixture was purified by preparative HPLC to afford after lyophilization the desired compound 1050 (74 mg, 13% yield) as a white solid. MS: (M+H)+; 742.0.

### Example 10

### Synthesis of Compound 1088

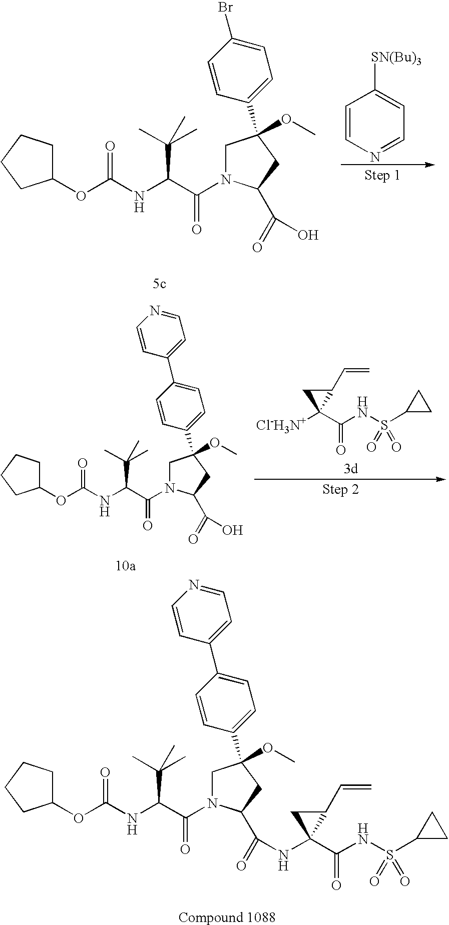

### Step 1:

To acid 5c (Example 5) (150 mg, 0.29 mmol) was added 4-tributylstannylpyridine (210 mg, 0.57 mmol) in anhydrous toluene (10 mL). This solution was bubbled with N₂ for 10 min before Pd(PPh₃)₄ (66 mg, 0.06 mmol) was added. The reaction was bubbled with N₂ for an additional 10 min, then heated to reflux for 6 h. The mixture was diluted with EtOAc (60 mL) and the organic layer washed with 1N NaOH (3×). The aqueous phase was acidified to pH˜4 with 4N HCl and extracted with dichloromethane (3×). The combined phases were dried over MgSO₄, filtered and concentrated to afford compound 10a as an oil (147 mg). This material was dried under high vacuum and used as is in the final coupling step.

### Step 2:

Acid 10a (147 mg, 0.28 mmol) was dissolved in anhydrous DMF (10 mL) and treated with HATU (139 mg, 0.36 mmol) and DIPEA (244 μL, 1.4 mmol). To this solution was added the amine hydrochloride salt 3d (0.36 mmol) previously neutralized with DIPEA (245 μL, 1.4 mmol) in DMF (2 mL). The reaction mixture was stirred at RT for 1 h. The crude reaction mixture was purified by preparative HPLC to afford after lyophilization the desired compound 1088 (18.2 mg, 9% yield) as a white solid. MS: (M+H)+; 736.0.

### Example 11

### Synthesis of Compound 1077

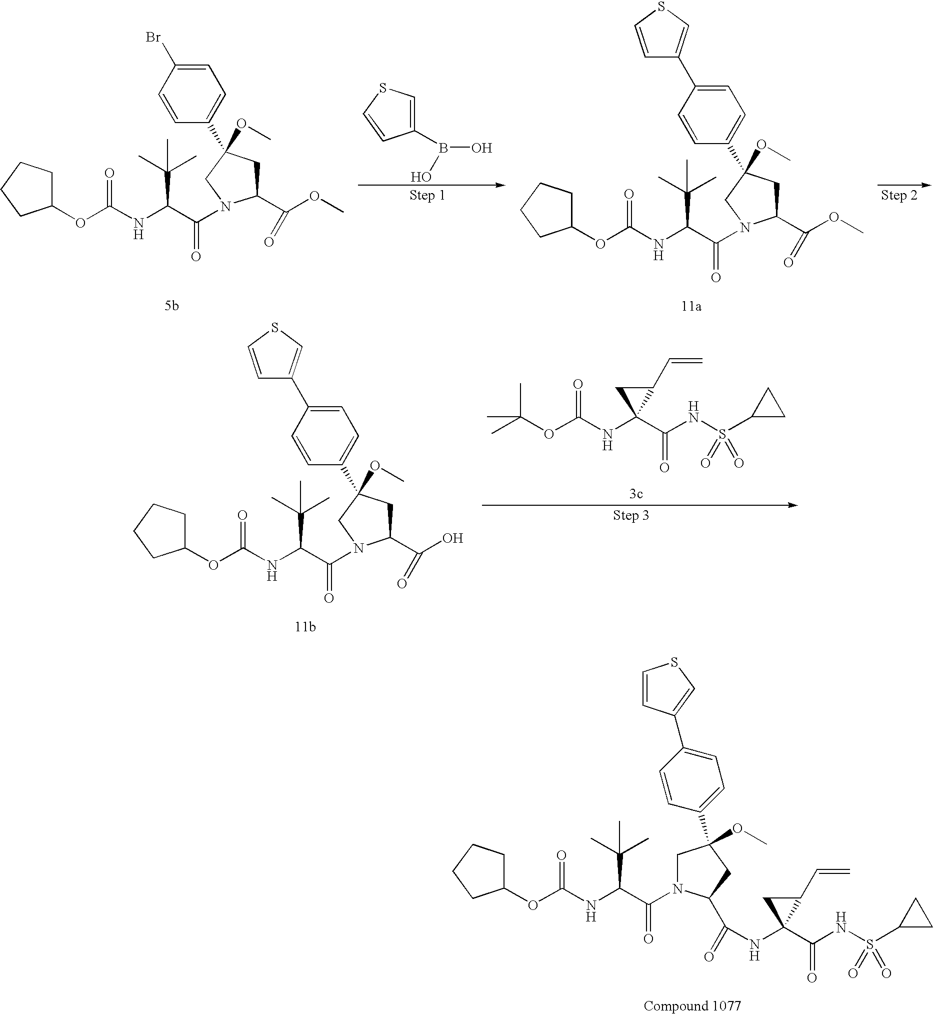

### Step 1:

A mixture of bromide 5b (105 mg, 0.19 mmol), 3-thiopheneboronic acid (75 mg, 0.59 mmol), Pd(PPh₃)₄ (9.7 mg, 0.01 mmol), DME (2 mL) and 2M Na₂CO₃ solution (0.78 mL, 1.56 mmol) in a flame dried flask was degassed with argon for 20 min. The mixture was heated at 90° C. for 14 h, and cooled to RT, then water was added and the mixture was extracted with EtOAc (3×). The combined organic layers were dried, filtered and concentrated to give a yellow oil 11a which was employed in the subsequent step without further purification. MS ES+=543.1.

### Step 2:

A solution of 1M NaOH (1.0 mL) was added to ester 11a (105 mg, 0.19 mmol) in THF (2 mL) and MeOH (1 mL) at RT. The mixture was allowed to stir for 14 h, concentrated and the crude product 11b was used in the subsequent step without further purification MS ES+=529.1.

### Step 3:

To the BOC-protected amino acid 3c (61 mg, 0.18 mmol) was added 3 mL of a 4M HCl/dioxane solution. The reaction was stirred at RT for 1 hr and the solvent was evaporated and the resulting solid placed under high vacuum for 1 h. The residue was dissolved in DMF (1.0 mL) and to it was added DIPEA (0.12 mL, 0.71 mmol). This solution was added to a solution of acid 11b (75 mg, 0.14 mmol) in DMF (1.5 mL) to which was added HATU (70 mg, 0.18 mmol), and the resulting mixture was allowed to stir at RT for 14 h. The mixture was filtered on Millex filter and directly purified by prep-HPLC (column: YMC; 50×20 mm I.D.; S-5 um). The relevant fractions were analyzed, pooled and lyophilized to yield the desired compound 1077 as a white lyophilized solid (11 mg, 10% yield for three steps). M.S. (electrospray): 739.3 (M−HOMe)⁻763.3 (M+H)⁺.

### Example 12

### Synthesis of Compound 1104

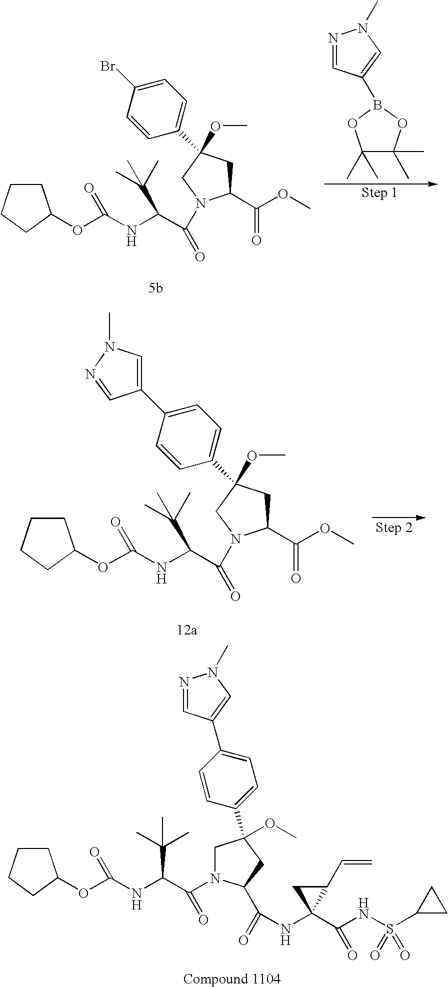

### Step 1:

A mixture of bromide 5b (105 mg, 0.19 mmol), 1-methyl-4-(4,4,5,5-tetramethyl-[1,3,2]dioxaborolan-2-yl)-1H-pyrazole (150 mg, 0.278 mmol), PdCl₂(dppf) (complex 1/1 with DCM) (45 mg, 0.028 mmol), DMSO (2.5 mL) and potassium acetate (79 mg, 0.834 mmol), in a flame dried flask, was degassed with argon and high vacuum for 20 min. The mixture was heated at 80° C. for 20 h, cooled to RT, acidified with 1 M HCl and extracted with EtOAc (3×). The combined organic layers were dried, filtered and concentrated to give compound 12a as a yellow oil which was used in the subsequent step without further purification. MS ES+=543.1.

### Step 2:

Using procedures analogous to those described in Example 10, steps 2 and 3, compound 12a was transformed to give compound 1104 as a beige lyophilized solid (38 mg, 21% yield). M.S. (electrospray): 737.3 (M−H)⁻.

### Example 13

### Synthesis of Compound 1121

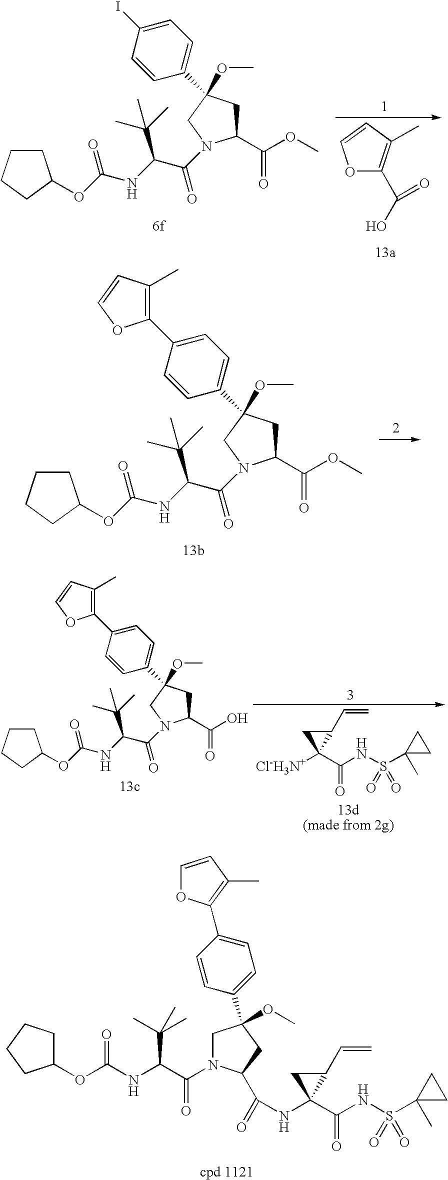

### Step 1:

In a vial suitable for microwave reactions were added the iodo 6f (300 mg, 0.51 mmol), the furoic acid 13a (194, mg, 1.54 mmol), potassium acetate (100 mg, 1.02 mmol), Bu₄N⁺Br⁻ ((165 mg, 0.51 mmol) and Pd[(tBu)₃P]₂ (52 mg, 0.1 mmol). DMF (4 ml) was then added. The vial was then capped and submitted directly to the microwave heating at 180° C. for 10 min. The mixture was directly purified by flash column chromatography to yield the desired product 13b (200 mg, 72% yield). ES+=509.4.

### Step 2:

LiOH (40 mg, 0.94 mmol) was added to ester 13b (102 mg, 0.19 mmol) in THF (1 mL), MeOH (90.5 mL) and water (0.5 mL) and stirred for 14 h at RT. Citric acid (20 mL) was added and extracted with EtOAc (3×20 mL). The combined organic was washed with brine, dried, filtered, concentrated and used in subsequent reaction without further purification.

### Step 3:

Acid 13c (48 mg, 0.09 mmol) was dissolved in anhydrous DMF (1.0 mL) and treated with HATU (45 mg, 0.12 mmol) and DIPEA (64 μL, 0.36 mmol). To this solution was added neutralized amine salt 13d (made from 2 g using the procedure described in example 3) (33 mg, 0.12 mmol) in DMF (1 mL). The reaction mixture was stirred at for 4 h. 1 M HCl was added to the mixture, and extracted with EtOAc (3×20 mL). The combined organic layer was dried, filtered and concentrated and then purified by preparative HPLC to give the desired compound 1121 (20 mg, 29% yield). MS: (M+H)⁺; 753.2 and (M−H)⁻; 751.2.

### Example 14

### Synthesis of Compound 2009

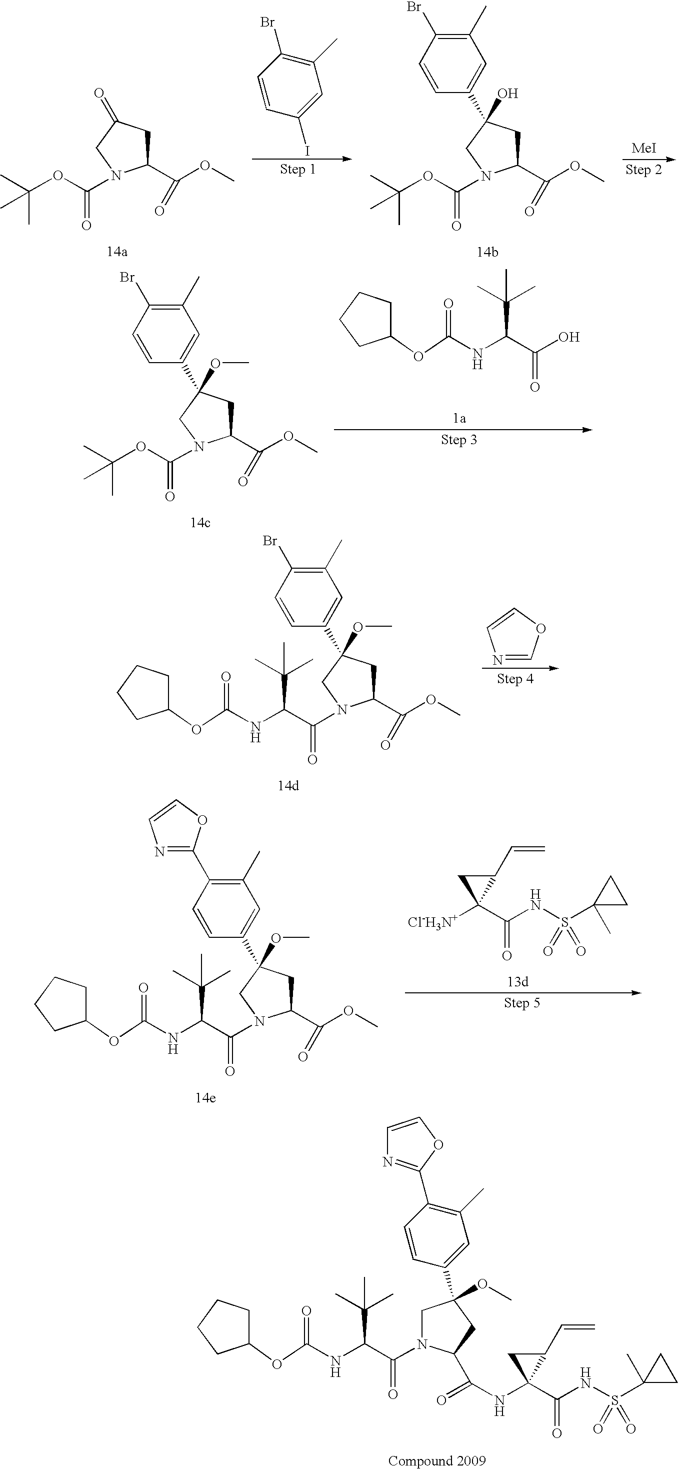

### Step 1:

In a dried flask containing freshly prepared CeCl₃ (1.53 g, 6.22 mmol) under an argon atmosphere was added anhydrous ether (16 mL). This suspension was cooled to 0° C. before the ketone 14a (1.51 g, 6.22 mmol) was added in ether (16 mL). This milky suspension was stirred at 0° C. for 2 h.

In a separate flask, Et₂O (10 mL) and THF (4 mL) was added to 2-bromo-5-iodotoluene (1.85 g, 6.22 mmol) and the mixture was cooled to −40° C. To this solution was added i-PrMgCl (2.0 M solution in Et₂O, 3.42 mL, 6.85 mmol) dropwise over ˜2 minutes to produce a yellow solution. The solution was stirred for a total of 40 minutes at −40° C. The ketone solution (above) was cooled to −50° C. and the above exchange reaction solution was added dropwise but rapidly via cannula over ˜2 minutes. When the addition was complete the cold bath was removed after 30 min and the reaction was allowed to warm to RT over another 45 minutes. TLC indicated the reaction was complete and the reaction was quenched by the addition of sat NH₄Cl (10 mL). The mixture was stirred rapidly for 5 minutes and then filtered through Celite. The filter cake was washed with 100 mL of EtOAc, transferred to a separatory funnel and the organic phase was washed twice with H₂O and brine, dried over MgSO₄, filtered and concentrated in vacuo to give a brownish oil (2.3 g). Purification by flash chromatography (30% EtOAc/hexanes) gave the desired compound 14b (0.78 g, 34% yield).

### Step 2:

The alcohol 14b (0.78 g, 1.9 mmol) was dissolved in anhydrous DMF with Mel (2.18 mL, 18.4 mL) and cooled to 0° C. before KH (340 mg, 4.3 mmol) was carefully added. After 1 h, the reaction was quenched carefully with water and extracted with EtOAc. The organic phase was washed with sat. brine and dried (MgSO₄) filtered and concentrated to give the pure ether 14c (856 mg, 91.5%). MS: (M+H)⁺; 428 and (MH+2)⁺; 430.

### Step 3:

To the BOC-protected amino acid 1a (728 mg, 1.7 mmol) was added 5 mL of a 4M HCl/dioxane solution. The reaction was stirred at RT for 1 hr and the solvent was evaporated and the resulting solid placed under high vacuum for 1 h. The residue was dissolved in DMF (10 mL) with HATU (765 mg, 2.01 mmol) and the acid 1a (468 mg, 1.93 mmol). To this solution was added DIPEA (1.53 mL, 8.75 mmol). The reaction was stirred at RT and was completed in 40 min. The mixture was quenched with water and extracted with EtOAc. The organic phase was washed with 10% HCl (aq), sat. NaHCO₃, and finally sat. brine (3×) before being dried (MgSO₄), filtered and concentrated to give a white solid 14d (941 mg, 100% yield).

### Step 4:

In a vial suitable for microwave reactions was added the dipeptide 14d (450 mg, 0.81 mmol), oxazole (267 μL, 4.07 mmol), potassium acetate (160 mg, 1.63 mmol), Bu₄N⁺Br⁻ (262 mg, 0.81 mmol), copper iodide (310 mg, 1.63 mmol), and Pd[(tBu)₃P]₂ (83 mg, 0.20 mmol) in DMF (14 ml). The vial was capped and submitted directly to the microwave conditions: Apparatus: Biotage Initiator Sixty, Absorption level: High, Run time: 10 min t=180° C. HPLC and LC-MS showed that the desired product was formed as two regioisomers 14e and 14f (close peaks by HPLC). To this crude mixture was added EtOAc (150 ml) which was washed with brine (3×), water, before being dried (MgSO₄), filtered and concentrated. The material was purified by CombiFlash (eluent=60% EtOAc/hexanes) to afford both regioisomers, 2-oxazole: 14e (138 mg, 31% yield) as a pale yellow solid [MS: (M−H)⁺; 542] and 5-oxazole: 14f (110 mg, 25% yield) as a pale yellow solid.

### Step 5:

The 2-oxazole dipeptide methyl ester 14e (138 mg, 0.25 mmol) was dissolved in THF/MeOH (8 mL/4 mL) and treated with 1N NaOH for 16 h. The mixture was acidified to pH ˜4 with 4N HCl and extracted with methylene chloride (3×). The combined organic phases were dried (MgSO₄), filtered and concentrated to dryness to afford the desired terminal acid (134 mg, 100%). MS: (M+H)⁺; 528.3 and (M−H)⁻; 526.2. The amine coupling partner 13d (0.17 mmol) was dissolved in DMF (1 mL) and treated with DIPEA (111 mL, 0.63 mmol) and added to a mixture of the acid (0.13 mmol) and HATU (63 mg, 0.17 mmol) in DMF (2 mL). The coupling was stirred at RT for 30 minutes before being filtered and purified by preparatory HPLC. The pure fractions were lyophilized to afford compound 2009 as a white amorphous solid (51.4 mg, 54% yield). M.S: (M+H)⁺; 754.2 and (M−H)⁻; 752.1.

### Example 15

### Synthesis of Compound 2001

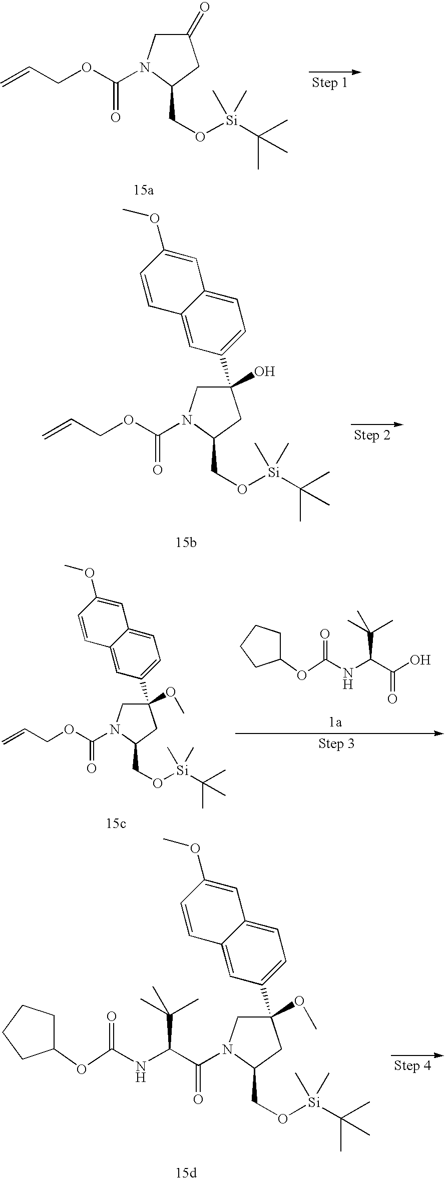

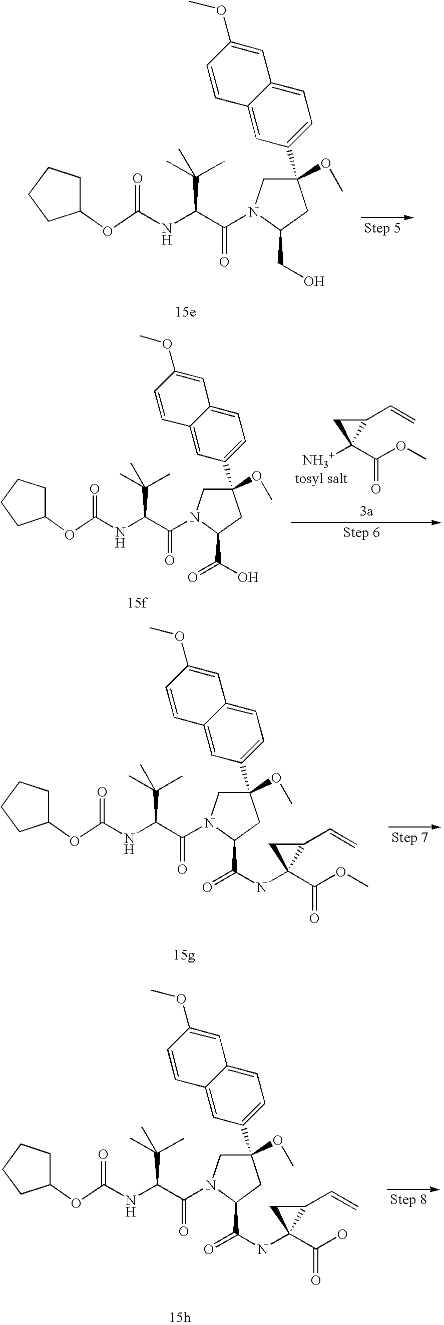

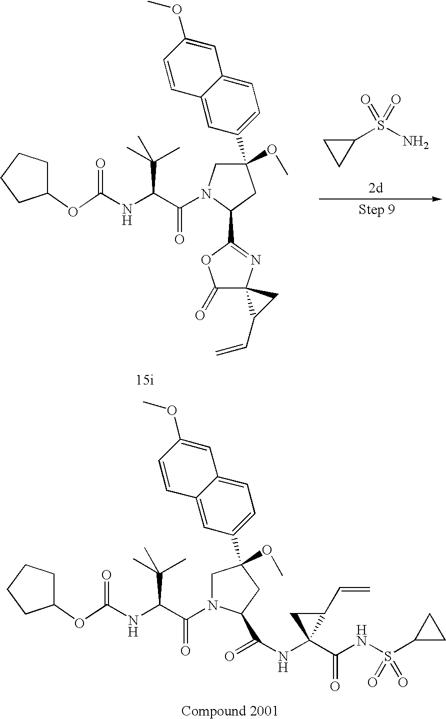

### Step 1:

To a solution of i-PrMgCl—LiCl (0.82M; 3.9 mL; 3.19 mmol), was added at RT and in one portion, 2-bromonaphthalene (780 mg; 3.19 mmol) dissolved in anhydrous THF (3.0 mL). The light yellow solution was stirred at a bath temperature of 45-48° C. for 3 h. Grignard reagent formation was verified by analytical HPLC and found to be in a 1: ˜2 ratio with the bromo starting material. The Grignard reagent was removed from the oil bath and the ketone 15a (500 mg; 1.60 mmol) dissolved in anhydrous THF (5.0 mL) was added immediately in a fast dropwise addition. The reaction mixture was stirred at RT overnight, then was diluted with saturated ammonium chloride and extracted with dichloromethane (3×). The combined extracts were washed with brine (1×), dried (MgSO₄), filtered and evaporated to dryness to provide the crude product 15b as a light yellow paste. Purification by flash chromatography (hexane:EtOAc; 95:5, then, 90:10) provided pure product 15b (99 mg; 13% yield).

### Step 2:

To an ice cooled solution of the hydroxy starting material 15b (99 mg; 0.21 mmol) in THF (2.0 mL) was added iodomethane (65 μL; 1.05 mmol) followed by hexane washed KH (10.52 mg; 0.26 mmol). The yellow mixture was stirred for 1 hour, after which by HPLC revealed the presence of only a small amount of desired product. Therefore, another 10.5 mg KH and 654 iodomethane was added and the reaction mixture allowed to stir for an additional 5 hrs to reveal no starting material by TLC (hexane:EtOAc; 8:2). The reaction mixture was diluted with water and extracted with dichloromethane (3×). The combined extracts were washed with brine (1×), dried (MgSO₄), filtered and evaporated to dryness to provide the product 15c as a yellow solid (95.4 mg; 94% yield)

### Step 3:

To compound 15c (95.4 mg: 0.196 mmol) dissolved in anhydrous THF (2.0 mL) was added 1,3-dimethylbarbituric acid (92.58 mg; 0.393 mmol) followed by triphenylphosphine tetrakis palladium (0) (69.5 mg; 0.06 mmol). The yellow solution was allowed to stir at RT and reaction was found to be complete after 4 hrs. The reaction mixture was evaporated to dryness to provide the free amine as an orange-red foam like gum. This gum was dissolved in dichloromethane (2.0 mL) and DIPEA (136.9 μL; 0.79 mmol) was added followed by compound 1a (Example 1A) (52.6 mg; 0.216 mmol) and HATU (89.6 mg; 0.236 mmol). The reaction was stirred overnight at RT and worked-up. The mixture was diluted with EtOAc, washed with sat'd NaHCO₃ (1×), 10% citric acid (2×), sat'd NaHCO₃ (2×), water (2×) and brine (1×), dried (MgSO₄), filtered and evaporated to dryness to provide the product 15d as a reddish brown foam (assume 0.196 mmol).

### Step 4:

To compound 15d (assume 0.196 mmol) dissolved in THF (1.0 mL) was added 1M TBAF (392 μL; 0.392 mmol) dissolved in THF (1.0 mL) and the reaction mixture was allowed to stir at RT for 3 hours, then evaporated to dryness to provide the crude material 15e as an orange oil. The crude material was purified by flash chromatography (hexane:EtOAc; 7:3, then, 6:4) to provide pure product 15e as an ivory foam (43.5 mg; 44% yield over 3 steps)

### Step 5:

The procedure described in Del Valle, J. R. et al, J. Org. Chem., 2003, 68(10), 3923-3931 (herein incorporated by reference) was used. Compound 15e (43.5 mg; 0.085 mmol) was dissolved in 1.5 mL of a 3:2 mixture of CH₃CN:NaH₂PO₄ buffer (pH 6.6; 0.67 M in H₂O). The solution was warmed to a bath temperature of 45° C. and TEMPO (1.4 mg; 0.008 mmol) was added followed by a simultaneous dropwise addition of a solution of sodium chlorite (80%; 19.3 mg; 0.214 mmol) in water (90 μL) and a solution of sodium hypochlorite (5.1 μL; conc. bleach at 6% solution) in water (90 μL), over 15 min. The bath was maintained at 45° C. and the reaction monitored by analytical HPLC. After 7.5 hrs only a small amount of an impurity (confirmed by LC-MS) was seen at the same t_{R} by HPLC. The reaction mixture was cooled to RT and a sat'd solution of Na₂SO₃ was added dropwise until a clear solution was obtained. The acetonitrile was evaporated and the aqueous layer was acidified to pH ˜3 with 1N HCl. The product was extracted with EtOAc (4×) and the combined extracts washed with brine (1×), dried (MgSO₄), filtered and evaporated to dryness to provide the product 15f as an off-white solid (assume 0.085 mmol).

### Step 6:

Compound 15f (assume 0.085 mmol) was dissolved in CH₃CN (1 mL), HATU (38.8 mg; 0.102 mmoles) added and the reaction mixture allowed to stir for 30 min. at RT. To this pre-formed activated ester was added the amine tosyl salt (3a, 31.96 mg; 0.102 mmoles) dissolved in CH₃CN (1 mL) with DIPEA (59.24; 0.34 mmoles). The mixture was stirred at RT overnight, then, evaporated to dryness. The residue was diluted with EtOAc and washed in succession with 10% citric acid (2×), water (2×), saturated NaHCO₃ (2×), water (2×) and brine (1×), dried (MgSO₄), filtered and evaporated to dryness to provide the product 15g as a light yellow foam (assume 0.085 mmol).

### Step 7:

Compound 15g was dissolved in THF (1 mL), MeOH (500 μL), water (500 μL). 1N NaOH (680 μL; 0.68 mmoles) was added and the reaction mixture stirred at RT for 4.5 hrs. The mixture was evaporated to near dryness, diluted with EtOAc, acidified to pH 3 with 1N HCl and the product extracted with EtOAc (3×). The combined extracts were washed with water (2×) and brine (1×), dried (MgSO₄), filtered and evaporated to provide product 15h as a white foam (46.5 mg; 86% yield 3 steps).

### Step 8:

Compound 15h (46.5 mg; 0.073 mmoles) was dissolved in CH₂Cl₂ (2 mLs) and TEA (33.6 μL; 0.241 mmoles) added. The reaction mixture was cooled in an ice bath and isobutylchloroformate (14.24; 0.11 mmoles) added dropwise. The mixture was stirred at 0° C. for 1 hr, then, the ice bath was removed and the reaction mixture stirred at RT overnight. Analytical HPLC showed the reaction to be complete and subsequently the mixture was loaded onto a flash column for purification (eluent:Hexane:EtOAc; 9:1, then, 8:2) to provide the azalactone product 15i as a colorless solid (18.9 mg, 42% yield).

### Step 9:

Using an oven dried flask, dissolve the cyclopropylsulfonamide (2d, 5.6 mg; 0.046 mmoles) in THF (1.0 mL). The light yellow solution was cooled to −15 to −20° C. and a 1M THF solution of LiHMDS (374; 0.037 mmoles) added in one shot. The resulting opaque mixture was stirred at the bath temperature for 5 min, then, at RT for 20 min. Subsequently, the reaction mixture was cooled to −10° C. and dropwise was added the azalactone (15i, 18.9 mg; 0.031 mmoles) dissolved in THF (1 mL). The reaction mixture was allowed to slowly warm to RT and left to stir overnight. The mixture was diluted with 1 N HCl to ˜pH 3 and the product extracted into EtOAc (3×). The combined EtOAc extracts were washed with water (2×) and brine (1×), dried (MgSO₄), filtered and evaporated to dryness to provide the crude product 2001 as a light yellow foam. The crude material was purified by preparatory HPLC (Reverse phase: YMC, Combiscreen ODS-AQ, 50×20 mm ID S-5 micron, 120 A; λ=220 nm) using a linear gradient and 0.06% TFA CH₃CN/H₂O from 2-100% CH₃CN. The fractions were analyzed by analytical HPLC (Reverse phase: YMC, Combiscreen ODS-AQ, 50×4.6 mm ID S-5 micron, 120 A; λ=220 nm), pure fractions were combined, concentrated and lyophilized to provide compound 2001 as a white amorphous solid (13 mg; 58% yield). MS: 737.3 (M−H)-707.2 (M-MeOH)⁺.

### Example 16

### NS3-NS4A Protease Assay

The enzymatic assay used to evaluate the present compound is described in WO 00/09543 and WO 00/59929.

### Example 17

### Cell-Based luciferase reporter HCV RNA Replication Assay

Representative compounds of the invention were tested for activity as inhibitors of hepatitis C virus RNA replication in cells expressing a stable subgenomic HCV replicon, using the assay described in WO 2005/028501.

Representative compounds of this invention are found to be active when evaluated in the preceding enzymatic and cell based assays.

### Example 18

### Specificity Assays

The specificity assays used to evaluate the selectivity of compounds according to this invention were performed as described in WO 00/09543 except that the assay buffer for the Elastase assay was comprised of 50 mM Tris-HCl pH 8, 0.25 M NaCitrate, 0.01% n-dodecyl β-d-maltoside, and 5.25% DMSO.

Representative compounds of formula (I) are found to be selective in that they do not show significant inhibition (no measurable activity at concentrations up to 30 μM) in the Human Leukocyte Elastase or Human Liver Cathepsin B assays.

### Tables of Compounds

The following tables list compounds representative of the invention. Representative compounds listed in Tables 1 and 2 below show unexpectedly good activity or activity below 50 nM when tested in the assays of Examples 16 and 17.

Retention times (t_{R}) for each compound were measured using the standard analytical HPLC conditions described in the Examples. As is well known to one skilled in the art, retention time values are sensitive to the specific measurement conditions. Therefore, even if identical conditions of solvent, flow rate, linear gradient, and the like are used, the retention time values may vary when measured, for example, on different HPLC instruments. Even when measured on the same instrument, the values may vary when measured, for example, using different individual HPLC columns, or, when measured on the same instrument and the same individual column, the values may vary, for example, between individual measurements taken on different occasions.

TABLE 1

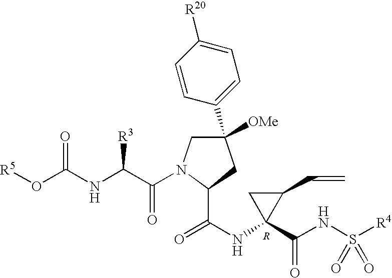

| | | | | | | t_{R} |
|---|---|---|---|---|---|---|
| Cpd | R⁵ | R³ | R²⁰ | R⁴ | (MH)⁺ | (min) |
| 1001 | 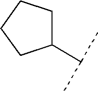 | 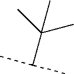 | 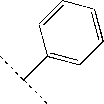 | 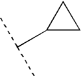 | 735.4 | 8.1 |
| 1002 | 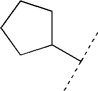 | 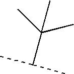 | 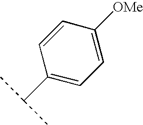 | 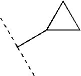 | 763.1 (M − H)⁻ | 8.3 |
| 1003 | 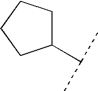 | 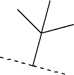 | 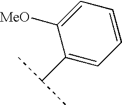 | 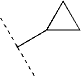 | 763.4 (M − H)⁻ | 8.4 |
| 1004 | 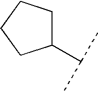 | 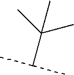 | 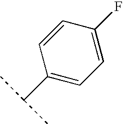 | 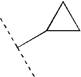 | 751.4 (M − H)⁻ | 8.4 |
| 1005 | 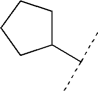 | 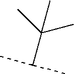 | 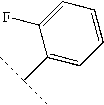 | 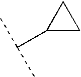 | 751.4 (M − H)⁻ | 8.4 |
| 1006 | 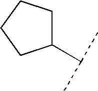 | 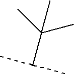 | 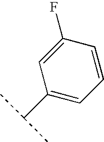 | 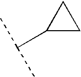 | 753.4 | 8.4 |
| 1007 | 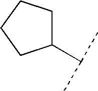 | 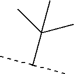 | 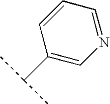 | 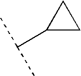 | 736.3 | 5.7 |
| 1008 | 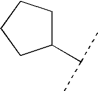 | 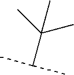 | 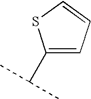 | 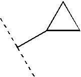 | 739.3 (M − H)⁻ | 8.1 |
| 1009 | 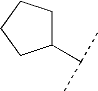 | 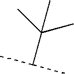 | 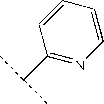 | 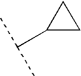 | 736.4 | 5.8 |
| 1010 | 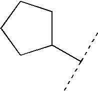 | 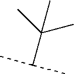 | 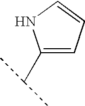 | 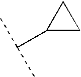 | 722.3 (M − H)⁻ | 7.6 |
| 1011 | 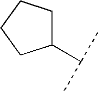 | 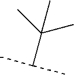 | 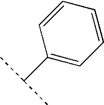 | 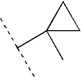 | 749.3 | 6.8 |
| 1012 | 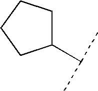 | 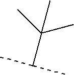 | 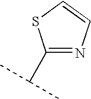 | 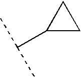 | 742.3 | 7.3 |
| 1013 | 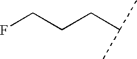 | 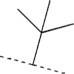 | 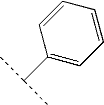 | 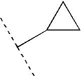 | 725.3 (M − H)⁻ | 6.1 |
| 1014 | 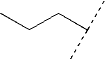 | 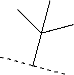 | 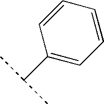 | 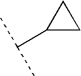 | 707.3 (M − H)⁻ | 6.4 |
| 1015 | 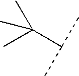 | 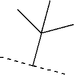 | 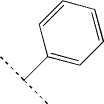 | 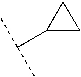 | 723.4 | 6.6 |
| 1016 | 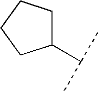 | 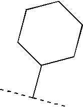 | 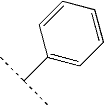 | 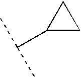 | 759.1 (M − H)⁻ | 6.9 |
| 1017 | 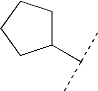 | 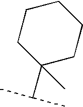 | 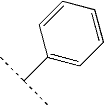 | 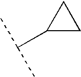 | 773.1 (M − H)⁻ | 7.2 |
| 1018 | 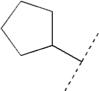 | 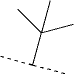 | 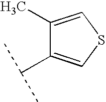 | 753.0 (M − H)⁻ | 8.2 | |
| 1019 | 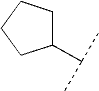 | 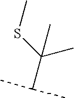 | 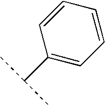 | 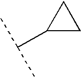 | 765.0 (M − H)⁻ | 7.5 |
| 1020 | 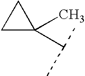 | 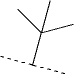 | 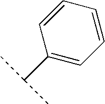 | 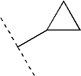 | 719.0 (M − H)⁻ | 7.1 |
| 1021 | 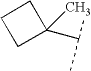 | 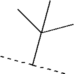 | 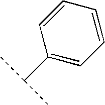 | 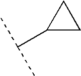 | 735.3 | 7.5 |
| 1022 | 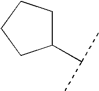 | 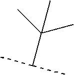 | 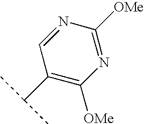 | 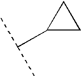 | 797.1 | 7.2 |
| 1023 | 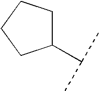 | 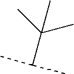 | 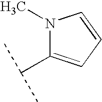 | 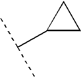 | 737.3 | 7.7 |
| 1024 | 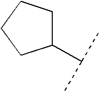 | 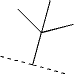 | 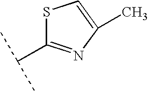 | 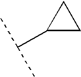 | 756.2 | 7.3 |
| 1025 | 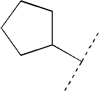 | 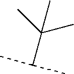 | 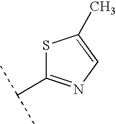 | 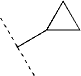 | 756.2 | 7.4 |
| 1026 | 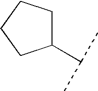 | 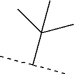 | 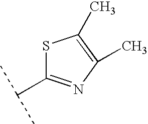 | 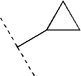 | 770.3 | 7.4 |
| 1027 | 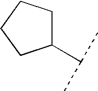 | 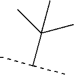 | 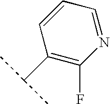 | 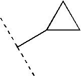 | 752.3 | 7.3 |
| 1028 | 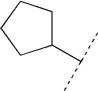 | 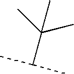 | 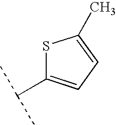 | 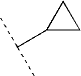 | 753.3 (M − H)⁻ | 8.1 |
| 1029 | 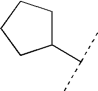 | 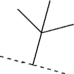 | 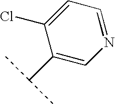 | 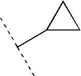 | 770.2 | 6.7 |
| 1030 | 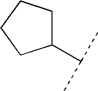 | 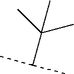 | 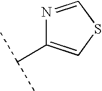 | 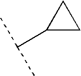 | 742.2 | 6.9 |
| 1031 | 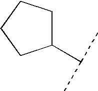 | 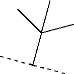 | 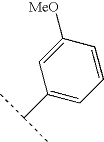 | 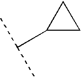 | 763.4 (M − H)⁻ | 8.4 |
| 1032 | 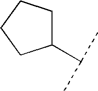 | 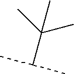 | 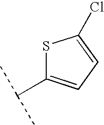 | 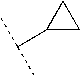 | 773.3 775.3 (M − H)⁻ | 8.3 |
| 1033 | 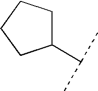 | 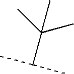 | 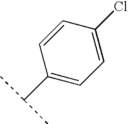 | 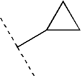 | 769.2 | 8.0 |
| 1034 | 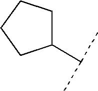 | 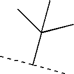 | 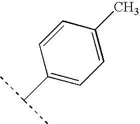 | 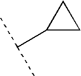 | 749.3 | 8.0 |
| 1035 | 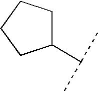 | 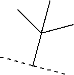 | 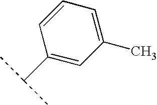 | 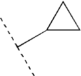 | 749.3 | 8.0 |
| 1036 | 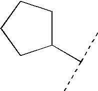 | 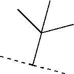 | 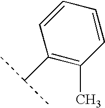 | 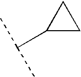 | 749.3 | 7.9 |
| 1037 | 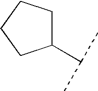 | 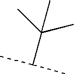 | 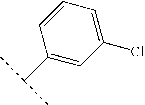 | 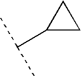 | 769.2 | 8.0 |
| 1038 | 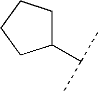 | 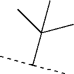 | 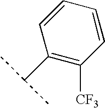 | 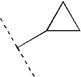 | 803.2 | 7.9 |
| 1039 | 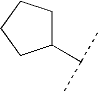 | 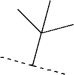 | 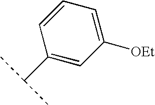 | 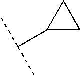 | 779.3 | 7.9 |
| 1040 | 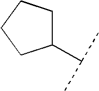 | 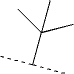 | 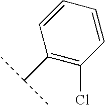 | 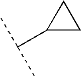 | 769.2 | 7.9 |
| 1041 | 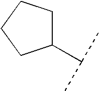 | 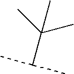 | 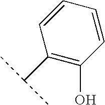 | 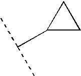 | 751.3 | 7.2 |
| 1042 | 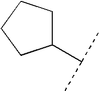 | 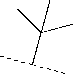 | 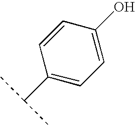 | 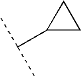 | 751.3 | 7.0 |
| 1043 | 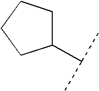 | 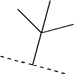 | 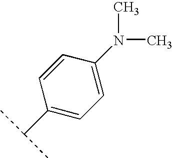 | 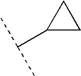 | 778.7 | 6.2 |
| 1044 | 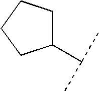 | 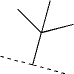 | 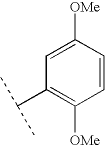 | 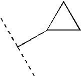 | 795.3 | 7.6 |
| 1045 | 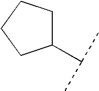 | 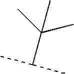 | 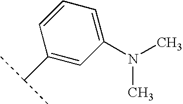 | 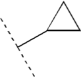 | 778.6 | 6.0 |
| 1046 | 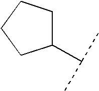 | 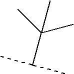 | 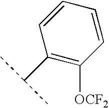 | 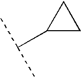 | 819.2 | 8.0 |
| 1047 | 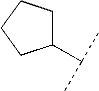 | 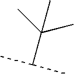 | 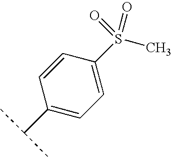 | 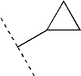 | 813.2 | 7.1 |
| 1048 | 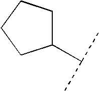 | 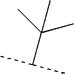 | 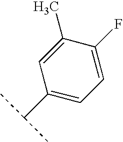 | 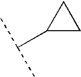 | 767.3 | 8.0 |
| 1049 | 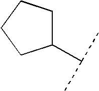 | 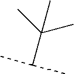 | 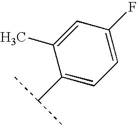 | 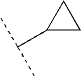 | 767.3 | 7.9 |
| 1050 | 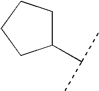 | 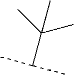 | 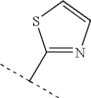 | 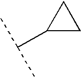 | 742.3 | 7.3 |
| 1051 | 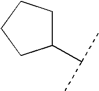 | 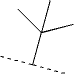 | 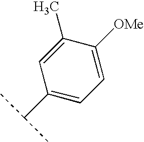 | 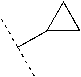 | 779.3 | 7.9 |
| 1052 | 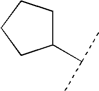 | 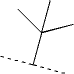 | 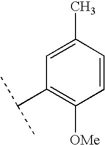 | 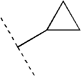 | 779.3 | 7.9 |
| 1053 | 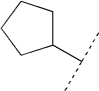 | 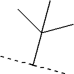 | 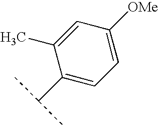 | 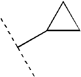 | 779.3 | 7.8 |
| 1054 | 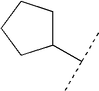 | 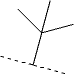 | 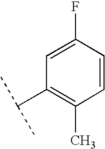 | 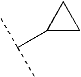 | 767.3 | 7.9 |
| 1055 | 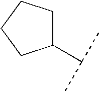 | 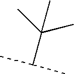 | 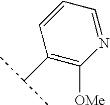 | 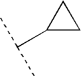 | 766.3 | 7.4 |
| 1056 | 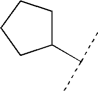 | 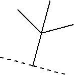 | 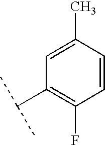 | 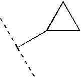 | 767.3 | 7.9 |
| 1057 | 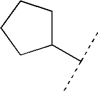 | 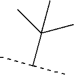 | 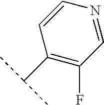 | 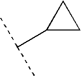 | 754.1 | 6.6 |
| 1058 | 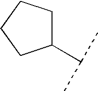 | 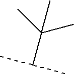 | 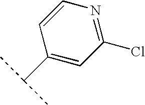 | 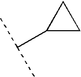 | 770.2 | 7.4 |
| 1059 | 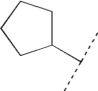 | 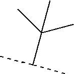 | 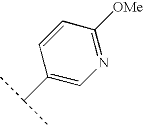 | 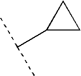 | 766.6 | 7.3 |
| 1060 | 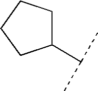 | 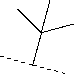 | 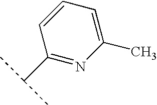 | 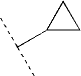 | 750.3 | 5.2 |
| 1061 | 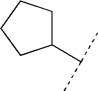 | 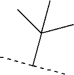 | 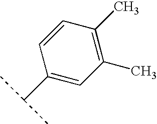 | 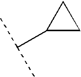 | 763.3 | 8.1 |
| 1062 | 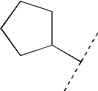 | 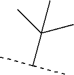 | 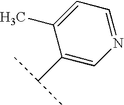 | 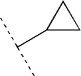 | 750.6 | 5.2 |
| 1063 | 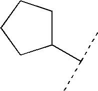 | 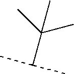 | 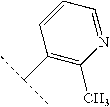 | 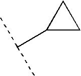 | 750.3 | 5.1 |
| 1064 | 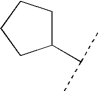 | 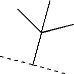 | 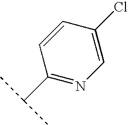 | 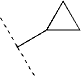 | 770.2 | 7.6 |
| 1065 | 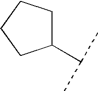 | 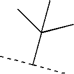 | 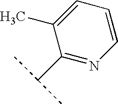 | 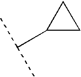 | 750.3 | 5.2 |
| 1066 | 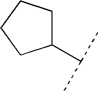 | 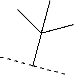 | 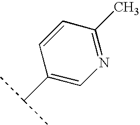 | 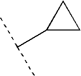 | 750.6 | 5.2 |
| 1067 | 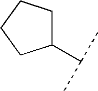 | 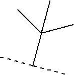 | 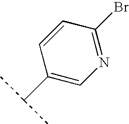 | 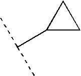 | 814.2 816.2 | 7.2 |
| 1068 | 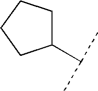 | 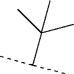 | 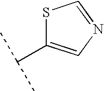 | 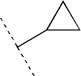 | 742.2 | 6.8 |
| 1069 | 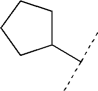 | 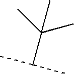 | 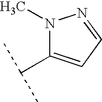 | 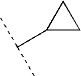 | 739.3 | 6.8 |
| 1070 | 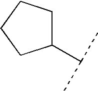 | 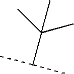 | 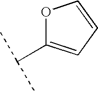 | 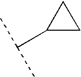 | 725.4 | 7.5 |
| 1071 | 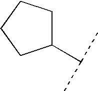 | 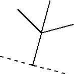 | 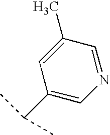 | 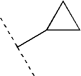 | 750.5 | 5.4 |
| 1072 | 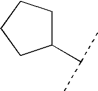 | 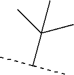 | 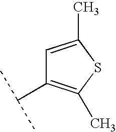 | 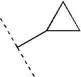 | 767.3 (M − H)⁻ | 8.2 |
| 1073 | 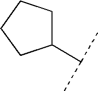 | 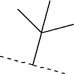 | 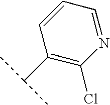 | 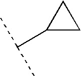 | 770.2 | 7.2 |
| 1074 | 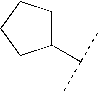 | 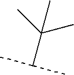 | 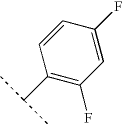 | 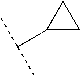 | 771.2 | 7.8 |
| 1075 | 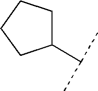 | 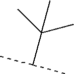 | 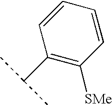 | 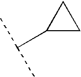 | 781.2 | 7.9 |
| 1076 | 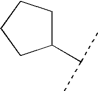 | 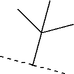 | 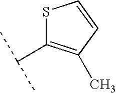 | 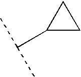 | 755.2 | 7.9 |
| 1077 | 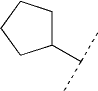 | 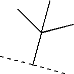 | 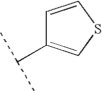 | 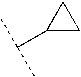 | 741.3 | 7.9 |
| 1078 | 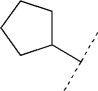 | 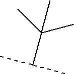 | 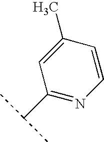 | 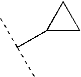 | 750.2 | 5.3 |
| 1079 | 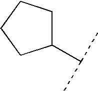 | 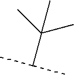 | 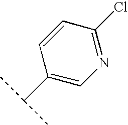 | 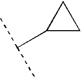 | 770.2 | 7.5 |
| 1080 | 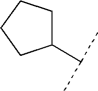 | 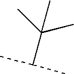 | 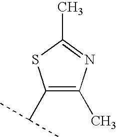 | 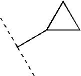 | 770.4 | 6.4 |
| 1081 | 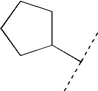 | 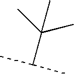 | 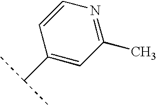 | 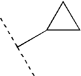 | 750.2 | 5.3 |
| 1082 | 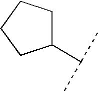 | 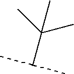 | 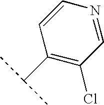 | 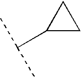 | 770.2 | 6.9 |
| 1083 | 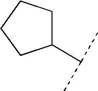 | 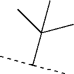 | 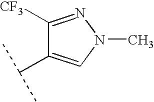 | 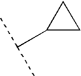 | 807.2 | 7.4 |
| 1084 | 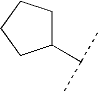 | 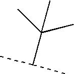 | 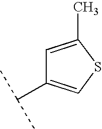 | 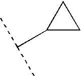 | 755.2 | 7.9 |
| 1085 | 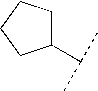 | 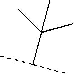 | 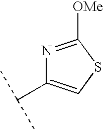 | 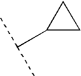 | 772.2 | 7.5 |
| 1086 | 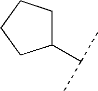 | 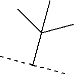 | 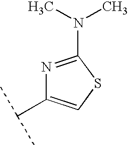 | 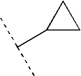 | 785.2 | 6.3 |
| 1087 | 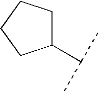 | 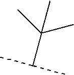 | 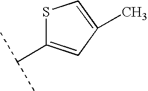 | 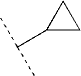 | 753.2 (M − H)⁻ | 7.7 |
| 1088 | 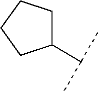 | 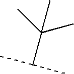 | 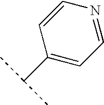 | 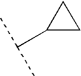 | 736.3 | 5.6 |
| 1089 | 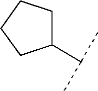 | 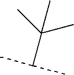 | 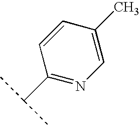 | 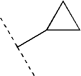 | 750.2 | 5.8 |
| 1090 | 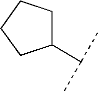 | 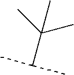 | 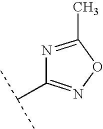 | 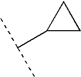 | 741.2 | 7.0 |
| 1091 | 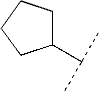 | 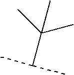 | 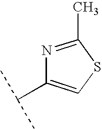 | 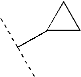 | 754.3 (M − H)⁻ | 7.1 |
| 1092 | 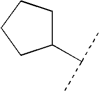 | 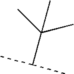 | 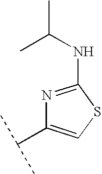 | 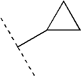 | 799.2 | 6.3 |
| 1093 | 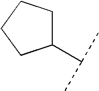 | 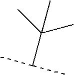 | 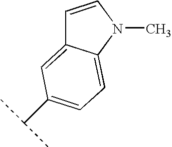 | 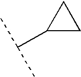 | 786.3 (M − H)⁻ | 7.4 |
| 1094 | 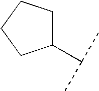 | 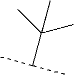 | 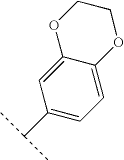 | 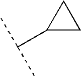 | 791.2 (M − H)⁻ | 7.3 |
| 1095 | 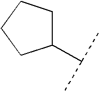 | 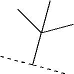 | 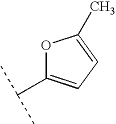 | 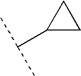 | 737.2 (M − H)⁻ | 7.7 |
| 1096 | 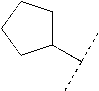 | 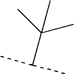 | 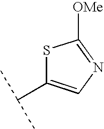 | 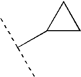 | 770.1 (M − H)⁻ | 7.3 |
| 1097 | 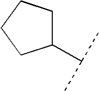 | 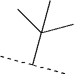 | 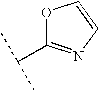 | 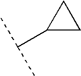 | 726.2 | 6.5 |
| 1098 | 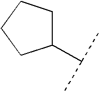 | 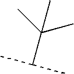 | 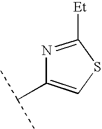 | 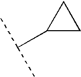 | 770.3 | 7.4 |
| 1099 | 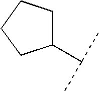 | 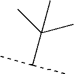 | 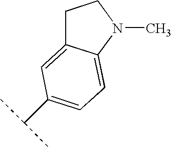 | 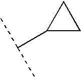 | 790.5 | 6.7 |
| 1100 | 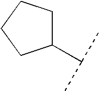 | 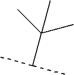 | 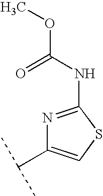 | 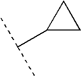 | 815.3 | 7.0 |
| 1101 | 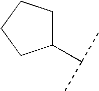 | 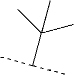 | 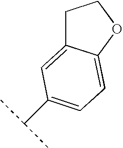 | 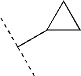 | 777.2 | 7.7 |
| 1102 | 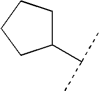 | 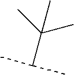 | 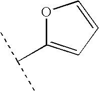 | 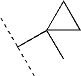 | 737.2 (M − H)⁻ | 7.5 |
| 1103 | 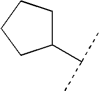 | 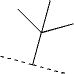 | 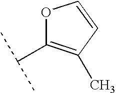 | 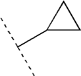 | 737.2 (M − H)⁻ | 7.5 |
| 1104 | 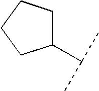 | 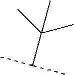 | 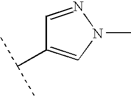 | 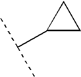 | 737.3 (M − H)⁻ | 7.1 |
| 1105 | 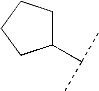 | 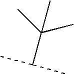 | 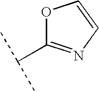 | 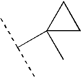 | 740.2 | 6.8 |
| 1106 | 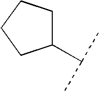 | 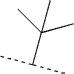 | 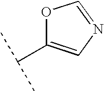 | 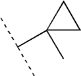 | 740.2 | 6.8 |
| 1107 | 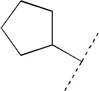 | 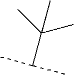 | 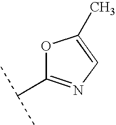 | 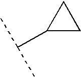 | 740.3 | 6.9 |
| 1108 | 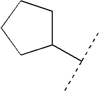 | 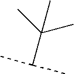 | 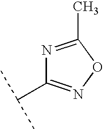 | 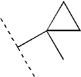 | 755.3 | 7.0 |
| 1109 | 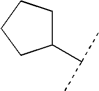 | 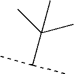 | 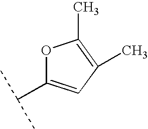 | 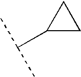 | 751.2 (M − H)⁻ | 7.8 |
| 1110 | 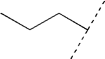 | 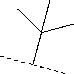 | 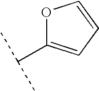 | 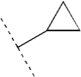 | 697.2 (M − H)⁻ | 6.7 |
| 1111 | 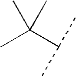 | 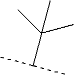 | 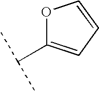 | 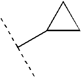 | 711.2 (M − H)⁻ | 7.0 |
| 1112 | 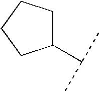 | 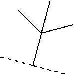 | 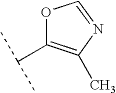 | 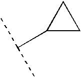 | 740.3 | 6.7 |
| 1113 | 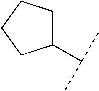 | 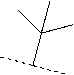 | 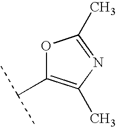 | 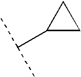 | 754.3 | 6.8 |
| 1114 | 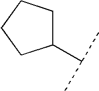 | 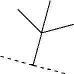 | 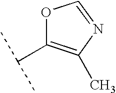 | 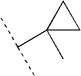 | 752.2 (M − H)⁻ | 6.9 |
| 1115 | 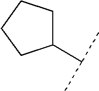 | 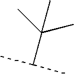 | 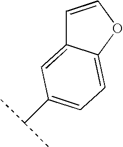 | 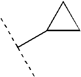 | 773.2 (M − H)⁻ | 7.7 |
| 1116 | 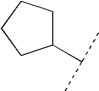 | 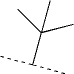 | 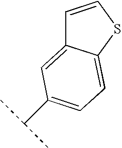 | 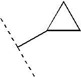 | 789.2 (M − H)⁻ | 7.9 |
| 1117 | 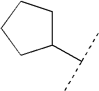 | 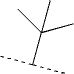 | 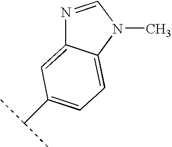 | 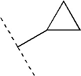 | 789.3 | 5.8 |
| 1118 | 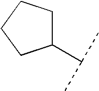 | 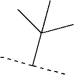 | 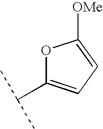 | 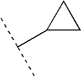 | 753.2 (M − H)⁻ | 7.3 |
| 1119 | 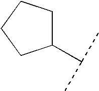 | 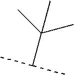 | 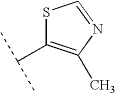 | 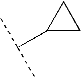 | 756.2 | 6.6 |
| 1120 | 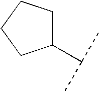 | 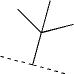 | 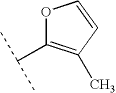 | 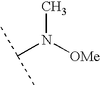 | 756.3 (M − H)⁻ | 7.5 |
| 1121 | 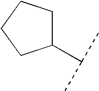 | 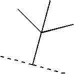 | 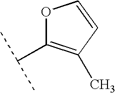 | 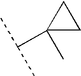 | 751.2 (M − H)⁻ | 7.5 |
| 1122 | 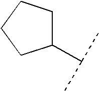 | 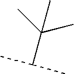 | 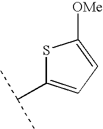 | 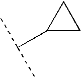 | 769.2 (M − H)⁻ | 7.5 |
| 1123 | 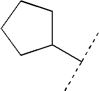 | 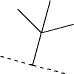 | 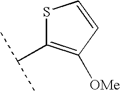 | 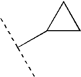 | 769.2 (M − H)⁻ | 7.4 |
| 1124 | 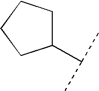 | 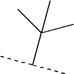 | 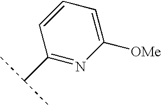 | 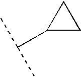 | 766.3 | 8.5 |
| 1125 | 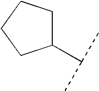 | 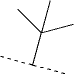 | 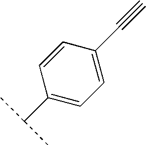 | 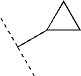 | 757.2 (M − H)⁻ | 8.4 |
| 1126 | 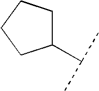 | 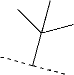 | 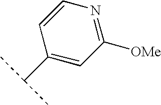 | 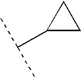 | 766.2 | 7.5 |
| 1127 | 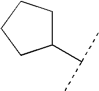 | 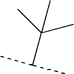 | 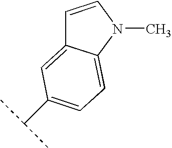 | 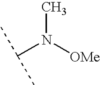 | 805.3 (M − H)⁻ | 8.5 |
| 1128 | 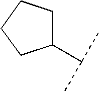 | 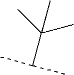 | 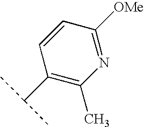 | 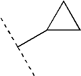 | 780.2 | 7.2 |
| 1129 | 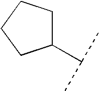 | 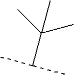 | 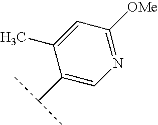 | 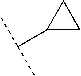 | 780.3 | 7.5 |
| 1130 | 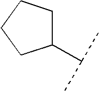 | 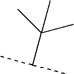 | 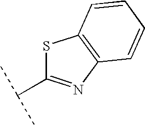 | 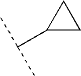 | 792.3 | 7.5 |
| 1131 | 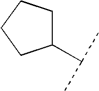 | 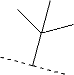 | 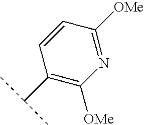 | 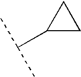 | 794.2 (M − H)⁻ | 8.5 |
| 1132 | 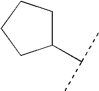 | 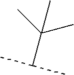 | 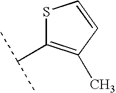 | 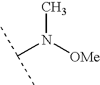 | 772.2 (M − H)⁻ | 8.5 |
| 1133 | 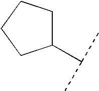 | 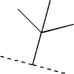 | 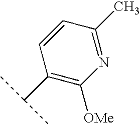 | 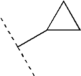 | 778.2 (M − H)⁻ | 8.2 |
| 1134 | 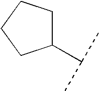 | 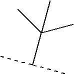 | 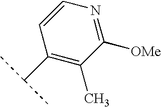 | 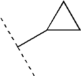 | 780.3 | 7.9 |

TABLE 2

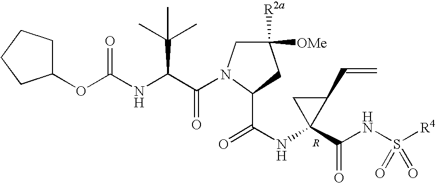

| | | | | t_{R} |
|---|---|---|---|---|
| Cpd | R^{2a} | R⁴ | (MH)⁺ | (min) |
| 2001 | 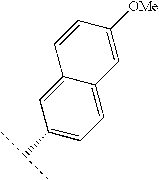 | 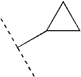 | 737.3 (M − H)⁻ | 7.7 |
| 2002 | 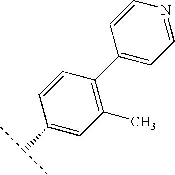 | 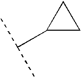 | 750.3 | 5.3 |
| 2003 | 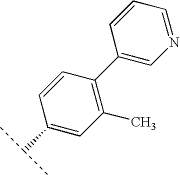 | 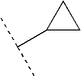 | 750.7 | 5.4 |
| 2004 | 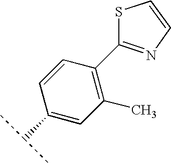 | 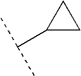 | 756.3 | 7.0 |
| 2005 | 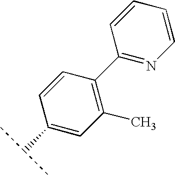 | 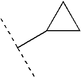 | 750.3 | 5.3 |
| 2006 | 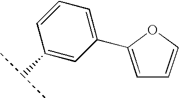 | 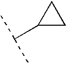 | 725.3 | 7.3 |
| 2007 | 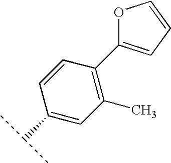 | 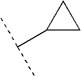 | 737.2 (M − H)⁻ | 7.5 |
| 2008 | 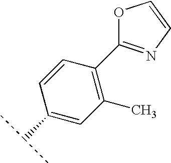 | 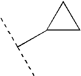 | 740.3 | 7.0 |
| 2009 | 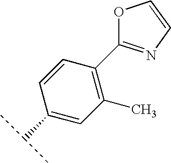 | 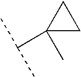 | 754.2 | 7.1 |
| 2010 | 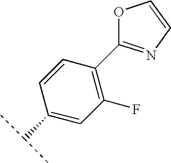 | 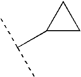 | 744.3 | 6.6 |
| 2011 | 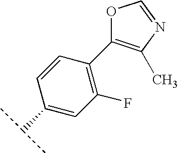 | 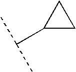 | 758.3 | 6.8 |
| 2012 | 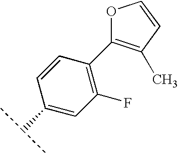 | 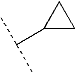 | 755.2 (M − H)⁻ | 7.5 |
| 2013 | 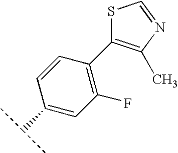 | 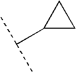 | 774.2 | 6.8 |

All of the documents cited herein are incorporated in to the invention as a reference, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A compound of formula (I): 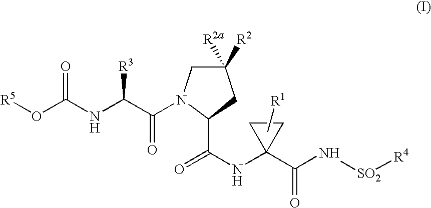
wherein:
R⁵ is selected from:
(i) (C₁₋₁₀)alkyl optionally substituted with one or more substituents each selected independently from —COOH, —COO(C₁₋₆)alkyl, —OH, halogen, —CN, —OC(═O)(C₁₋₆)alkyl, —O(C₁₋₆)alkyl, —NH₂, —NH(C₁₋₆)alkyl, —N((C₁₋₆)alkyl)₂, —C(═O)NH₂, —C(═O)NH(C₁₋₆)alkyl and —C(═O)N((C₁₋₆)alkyl)₂; and
(ii) (C₃₋₇)cycloalkyl, (C₃₋₇)cycloalkenyl, (C₃₋₇)cycloalkyl-(C₁₋₄)alkyl- or (C₃₋₇)cycloalkenyl-(C₁₋₄)alkyl-, each optionally substituted with one or more substituents each selected independently from (C₁₋₆)alkyl, (C₂₋₆)alkenyl, (C₂₋₆)alkynyl, —COOH, —COO(C₁₋₆)alkyl, —OH, —O(C₁₋₆)alkyl, —CN, —NH₂, —NH(C₁₋₆)alkyl, —N((C₁₋₆)alkyl)₂, —C(═O)NH₂, —C(═O)NH(C₁₋₆)alkyl and —C(═O)N((C₁₋₆)alkyl)₂;
R³ is (C₁₋₈)alkyl, (C₃₋₇)cycloalkyl or (C₃₋₇)cycloalkyl-(C₁₋₃)alkyl-, each optionally substituted with one or more substituents each independently selected from (C₁₋₆)alkyl, (C₂₋₆)alkenyl, (C₂₋₆)alkynyl, halogen, cyano, —OR³⁰, —SR³⁰, —C(═O)OR³⁰, —C(═O)NH₂, —C(═O)NH(C₁₋₆)alkyl, C(═O)N((C₁₋₆)alkyl)₂, —NH₂, —NH(C₁₋₆)alkyl, —N((C₁₋₆)alkyl)₂, aryl, and aryl(C₁₋₆)alkyl-, wherein R³⁰ is H, (C₁₋₆)alkyl, aryl, or aryl(C₁₋₆)alkyl-;
R² is —O(C₁₋₆)alkyl;
R^{2a} is 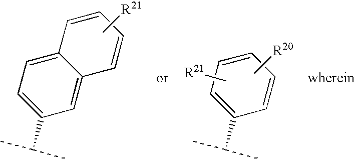
R²⁰ is selected from aryl and Het, each optionally substituted with one or more substituents each independently selected from halogen, cyano, (C₁₋₆)alkyl, (C₁₋₆)haloalkyl, —O(C₁₋₆)alkyl, —S(C₁₋₆)alkyl, —OH, —SH, —NH₂, —NH(C₁₋₆)alkyl, —N((C₁₋₆)alkyl)₂, —NHC(═O)(C₁₋₆)alkyl, —C(═O)NH₂, —C(═O)NH(C₁₋₆)alkyl, —C(═O)N((C₁₋₆)alkyl)₂, —COOH, —C(═O)O(C₁₋₆)alkyl and SO₂(C₁₋₆)alkyl; and
R²¹ is one to four substituents each independently selected from H, halogen, (C₁₋₆)alkyl, and —O(C₁₋₆)alkyl;
R¹ is (C₁₋₆)alkyl or (C₂₋₆)alkenyl; each of said (C₁₋₆)alkyl, (C₂₋₆)alkenyl being optionally substituted with from one to three halogen substituents; and
R⁴ is (C₃₋₇)cycloalkyl; said (C₃₋₇)cycloalkyl being optionally substituted with (C₁₋₆)alkyl; or
R⁴ is —N(R^{N2})R^{N1}, wherein R^{N1} and R^{N2} are each independently selected from H, (C₁₋₆)alkyl and —O—(C₁₋₆)alkyl;
wherein Het is defined as a 3- to 7-membered heterocycle having 1 to 4 heteroatoms each independently selected from O, N and S, which may be saturated, unsaturated or aromatic, and which is optionally fused to at least one other cycle to form a 4- to 14-membered heteropolycycle having wherever possible 1 to 5 heteroatoms, each independently selected from O, N and S, said heteropolycycle being saturated, unsaturated or aromatic;
or a diastereoisomer or tautomer thereof; or a salt thereof.

2. The compound according to claim 1, wherein the R¹ substituent is selected from: (C₁₋₄)alkyl or (C₂₋₄)alkenyl.

3. The compound according to claim 2, wherein R¹ is (C₁₋₃)alkyl or (C₂₋₄)alkenyl.

4. The compound according to claim 3, wherein R¹ is (C₂₋₃)alkenyl.

5. The compound according to claim 4, wherein R¹ is —CH═CH₂ (vinyl).

6. The compound according to claim 1, wherein R² is selected from: —OMe; —OEt; —OPr; —OButyl; —OPentyl and —OHexyl.

7. The compound according to claim claim 6, wherein R² is —OMe; —OEt; —O-nPr; or —O-iPr.

8. The compound according to claim 7, wherein R² is —OMe or —OEt.

9. The compound according to claim claim 8, wherein R² is OMe.

10. The compound according to claim 1, wherein the R^{2a} substituent is selected from: 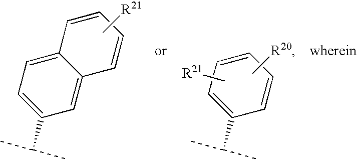
R²⁰ is selected from: phenyl and Het, each optionally substituted with one or more substituents each independently selected from halogen, (C₁₋₆)alkyl, (C₁₋₆)haloalkyl, —O(C₁₋₆)alkyl, —S(C₁₋₆)alkyl, —OH, —SH, —NH₂, —NH(C₁₋₆)alkyl, —N((C₁₋₆)alkyl)₂, and —NHC(═O)(C₁₋₆)alkyl; and
R²¹ is selected from: one to four substituents each independently selected from H, halogen, (C₁₋₆)alkyl and —O(C₁₋₆)alkyl.

11. The compound according to claim 10, wherein R^{2a} is 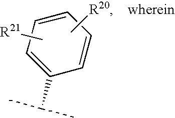
R²⁰ is phenyl or Het, each optionally substituted with one or more substituents each independently selected from halogen, (C₁₋₄)alkyl, (C₁₋₄)haloalkyl, —O(C₁₋₄)alkyl, —S(C₁₋₄)alkyl, —OH, —SH, —NH₂, —NH(C₁₋₃)alkyl, —N((C₁₋₃)alkyl)₂, and —NHC(═O)(C₁₋₃)alkyl; and
R²¹ is one to three substituents each independently selected from H, halogen, and (C₁₋₃)alkyl.

12. The compound according to claim 11 wherein R^{2a} is 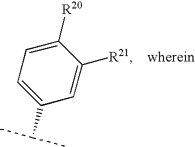
R²⁰ is phenyl and Het, each optionally substituted with one or two substituents each independently selected from Cl, F, Br, Me, Et, MeO, EtO, MeS, and EtS;
wherein said Het is selected from: 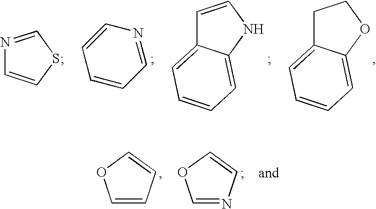
R²¹ is a substituent independently selected from: H, F or Me.

13. The compound according to claim 12, wherein the R³ substituent is selected from: (C₁₋₈)alkyl or (C₃₋₇)cycloalkyl, each optionally substituted with one substituent selected from: (C₁₋₆)alkyl, halogen, —SR³⁰, wherein R³⁰ is H or (C₁₋₆)alkyl.

14. The compound according to claim 13, wherein R³ is (C₁₋₈)alkyl optionally substituted with —S(C₁₋₆)alkyl; or (C₃₋₇)cycloalkyl optionally substituted with (C₁₋₆)alkyl.

15. The compound according to claim 14, wherein R³ is(C₁₋₄alkyl; or (C₆)cycloalkyl.

16. The compound according to claim 15, wherein R³ is tert-butyl.

17. The compound according to claim 1, wherein the R⁴ substituent is selected from: (C₃₋₇)cycloalkyl; said (C₃₋₇)cycloalkyl being optionally substituted with (C₁₋₆)alkyl; or R⁴ is —NHR^{N1}, wherein R^{N1} is H or (C₁₋₆)alkyl.

18. The compound of according to claim 17, wherein R⁴ is (C₃₋₆)cycloalkyl optionally substituted with (C₁₋₆)alkyl.

19. The compound according to claim 18, wherein R⁴ is (C₃₋₄cycloalkyl optionally substituted with methyl.

20. The compound according to claim 19, wherein R⁴ is cyclopropyl.

21. The compound according to claim 1, wherein R⁵ is (C₁₋₁₀)alkyl optionally substituted with one or more halogen; or (C₃₋₇)cycloalkyl optionally substituted with one or more (C₁₋₆)alkyl.

22. The compound according to claim 21, wherein R⁵ is (C₁₋₆)alkyl optionally substituted with fluoro; or (C₃₋₅)cycloalkyl optionally substituted with methyl.

23. The compound according to claim 22, wherein R⁵ is (C₃₋₄alkyl; or (C₃₋₅)cycloalkyl.

24. The compound according to claim 23, wherein R⁵ is tert-butyl or cyclopentyl.

25. A compound of formula (I): 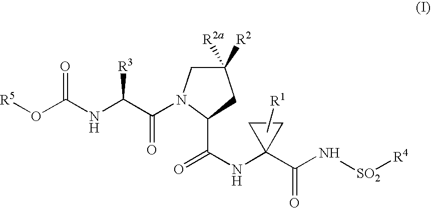
wherein R¹ is selected from: (C₁₋₄)alkyl or (C₂₋₄)alkenyl;
R² is —OMe; —OEt; —OPr; —OButyl; —OPentyl or —OHexyl;
R^{2a} is selected from: 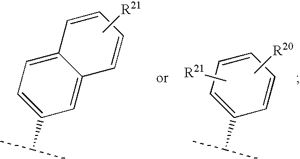
wherein R²⁰ is phenyl or Het, each optionally substituted with one or more substituents each independently selected from halogen, (C₁₋₆)alkyl, (C₁₋₆)haloalkyl, —O(C₁₋₆)alkyl, —S(C₁₋₆)alkyl, —OH, —SH, —NH₂, —NH(C₁₋₆)alkyl, —N((C₁₋₆)alkyl)₂, and —NHC(═O)(C₁₋₆)alkyl; and
R²¹ is one to four substituents each independently selected from H, halogen, (C₁₋₆)alkyl and —O(C₁₋₆)alkyl;
R³ is (C₁₋₈)alkyl or (C₃₋₇)cycloalkyl, each optionally substituted with one substituent selected from: (C₁₋₆)alkyl, halogen, —SR³⁰, wherein R³⁰ is H or (C₁₋₆)alkyl;
R⁴ is (C₃₋₇)cycloalkyl; said (C₃₋₇)cycloalkyl being optionally substituted with (C₁₋₆)alkyl; or R⁴ is NHR^{N1}, wherein R^{N1} is H or (C₁₋₆)alkyl; and
R⁵ is (C₁₋₁₀)alkyl optionally substituted with one or more halogen; or (C₃₋₇)cycloalkyl optionally substituted with one or more (C₁₋₆)alkyl;
wherein Het is defined as a 3- to 7-membered heterocycle having 1 to 4 heteroatoms each independently selected from O, N and S, which may be saturated, unsaturated or aromatic, and which is optionally fused to at least one other cycle to form a 4- to 14-membered heteropolycycle having wherever possible 1 to 5 heteroatoms, each independently selected from O, N and S, said heteropolycycle being saturated, unsaturated or aromatic;
or a diastereoisomer or tautomer thereof; or a salt thereof.

26. The compound according to claim 25, wherein R¹ is (C₁₋₃)alkyl or (C₂₋₄)alkenyl; R² is —OMe; —OEt; —O-nPr; or —O-iPr; R^{2a} is: 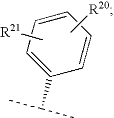
wherein R²⁰ is: phenyl and Het, each optionally substituted with one or more substituents each independently selected from halogen, (C₁₋₄)alkyl, (C₁₋₄haloalkyl, —O(C₁₋₄)alkyl, —S(C₁₋₄)alkyl, —OH, —SH, —NH₂, —NH(C₁₋₃)alkyl, —N((C₁₋₃)alkyl)₂, and —NHC(═O)(C₁₋₃)alkyl; and
R²¹ is one to three substituents each independently selected from H, halogen, and (C₁₋₃)alkyl;
R³ is (C₁₋₈)alkyl optionally substituted with —S(C₁₋₆)alkyl; or (C₃₋₇)cycloalkyl optionally substituted with (C₁₋₆)alkyl;
R⁴ is (C₃₋₆)cycloalkyl optionally substituted with (C₁₋₆)alkyl; and
R⁵ is (C₁₋₆)alkyl optionally substituted with fluoro; or (C₃₋₅)cycloalkyl optionally substituted with methyl.

27. The compound according to claim 26, wherein R¹ is (C₂₋₃)alkenyl; R² is —OMe or —OEt; R^{2a} is: 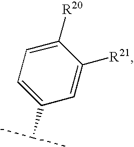
wherein R²⁰ is phenyl and Het, each optionally substituted with one or two substituents each independently selected from Cl, F, Br, Me, Et, MeO, EtO, MeS, and EtS;
wherein said Het is selected from: 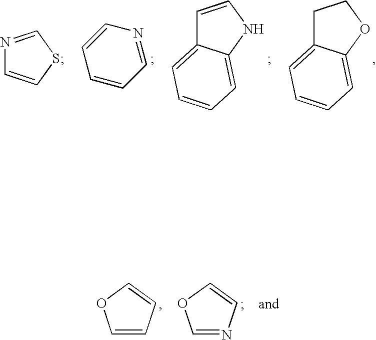
R²¹ is a substituent independently selected from: H, F or Me;
R³ is (C₁₋₄)alkyl; or (C₆)cycloalkyl;
R⁴ is (C₃₋₄)cycloalkyl optionally substituted with methyl; and
R⁵ is (C₃₋₄)alkyl; or (C₃₋₅)cycloalkyl.

28. The compound according to claim 27, wherein R¹ is CH═CH₂ (vinyl); R² is OMe; R³ is tert-butyl; R⁴ is cyclopropyl; and R⁵ is tert-butyl or cyclopentyl.

29. The compound according to claim 1, or a pharmaceutically acceptable salt thereof, as a medicament.

30. A pharmaceutical composition comprising a therapeutically effective amount of a compound of formula (I) according to claim 1, or a pharmaceutically acceptable salt thereof; and one or more pharmaceutically acceptable carriers.

31. The pharmaceutical composition according to claim 30 additionally comprising at least one other antiviral agent.

32. Use of a pharmaceutical composition according to claim 30 as for the treatment of a hepatitis C viral infection in a mammal having or at risk of having the infection.

33. A method of treating a hepatitis C viral infection in a mammal having or at risk of having the infection, the method comprising administering to the mammal a therapeutically effective amount of a compound of formula (I) according to claim 1, a pharmaceutically acceptable salt thereof, or a pharmaceutical composition according to claim 30.

34. A method of treating a hepatitis C viral infection in a mammal having or at risk of having the infection, the method comprising administering to the mammal a therapeutically effective amount of a combination of a compound of formula (I) according to claim 1, or a pharmaceutically acceptable salt thereof, and at least one other antiviral agent; or a pharmaceutical composition according to claim 30.

35. Use of a compound of formula (I) according to claim 1, or a pharmaceutically acceptable salt thereof, for the treatment of a hepatitis C viral infection in a mammal having or at risk of having the infection.

36. Use of a compound of formula (I) according to claim 1, or a pharmaceutically acceptable salt thereof, for the manufacture of a medicament for the treatment of a hepatitis C viral infection in a mammal having or at risk of having the infection.

37. An article of manufacture comprising a composition effective to treat a hepatitis C viral infection; and packaging material comprising a label which indicates that the composition can be used to treat infection by the hepatitis C virus; wherein the composition comprises a compound of formula (I) according to claim 1 or a pharmaceutically acceptable salt thereof.

38. A method of inhibiting the replication of hepatitis C virus comprising exposing the virus to an effective amount of the compound of formula (I) according to claim 1, or a salt thereof, under conditions where replication of hepatitis C virus is inhibited.

39. Use of a compound of formula (I) according to claim 1 or a salt thereof, to inhibit the replication of hepatitis C virus.
